(19) United States
(12) Patent Application Publication
(10) Pub. No.: US 2025/0222109 A1
(43) Pub. Date: Jul. 10, 2025

(54) STRUCTURED LIPID COMPOSITIONS

(71) Applicants: Universität Bern, Bern (CH); Universität Zürich, Zurich (CH)

(72) Inventors: Simone Aleandri, Bern (CH); Marianna Carone, Bern (CH); Robert Gaultney, Bern (CH); Philippe Krebs, Wohlen bei Bern (CH); Paola Luciani, Bern (CH); Gerhard Rogler, Benglen (CH); Marianne Spalinger, Staufen (CH); Rafaela Gazzi, Bern (CH)

(21) Appl. No.: 19/089,624

(22) Filed: Mar. 25, 2025

Related U.S. Application Data
(63) Continuation of application No. PCT/EP2023/076606, filed on Sep. 26, 2023.

(30) Foreign Application Priority Data
Sep. 26, 2022 (EP) 22197842.2

(51) Int. Cl. A61K 47/14; A61K 9/00; A61K 31/4174; A61K 31/436; A61K 31/519; A61K 31/58; A61K 31/606; A61P 1/04

(52) U.S. Cl. CPC A61K 47/14; A61K 9/0031; A61K 31/4174; A61K 31/436; A61K 31/519; A61K 31/58; A61K 31/606; A61P 1/04

(57) ABSTRACT

The present invention relates to a composition for use in treating a condition of the lower gastrointestinal tract, e.g. ulcerative colitis, wherein the composition comprises: a) a carrier comprising: a1) water in an amount of more than 10% to 30% by weight of the carrier; and a2) monoacylglycerol lipid in an amount of 70% to 90% by weight of the carrier, wherein the monoacylglycerol lipid comprises monolinolein or monoolein, or combinations thereof; and b) a pharmaceutically active agent, wherein the composition forms a lipid cubic phase at a temperature of 36° C. to 39° C. The composition is particularly suitable for rectal administration, for example, as an enema. Also disclosed is a composition, methods for preparing the compositions, and kits for making the compositions.

### CROSS-REFERENCE TO RELATED APPLICATION

This application is a continuation of International Application No. PCT/EP2023/076606, with an international filing date of Sep. 26, 2023, which claims the benefit of and priority to European Patent Application No. 22197842.2, filed on Sep. 26, 2022, the entire contents of which are incorporated by reference herein.

### FIELD OF THE INVENTION

The present invention relates to a composition for use in treating a condition of the lower gastrointestinal tract, e.g. ulcerative colitis, wherein the composition comprises: a) a carrier comprising: a1) water in an amount of more than 10% to 30% by weight of the carrier; and a2) monoacylglycerol lipid in an amount of 70% to 90% by weight of the carrier, wherein the monoacylglycerol lipid comprises monolinolein or monoolein, or combinations thereof; and b) a pharmaceutically active agent, wherein the composition forms a lipid cubic phase at a temperature of 36° C. to 39° C. The composition is particularly suitable for rectal administration, for example, as an enema. Also disclosed is a composition, methods for preparing the compositions, and kits for making the compositions.

### BACKGROUND OF THE INVENTION

Ulcerative colitis (UC) is a chronic remitting-relapsing inflammatory disorder of the large intestine, involving colonic and rectal mucosa.¹ Clinically, 75% of patients suffer from left sided colitis or proctitis, but the inflammation can spread upward in a continuous manner and involve the colon either partially or entirely.²

There is no known cure for ulcerative colitis and the chronic relapse and remission often results in patient disability.^{1,2} All treatments currently recommended by the European Crohn's and Colitis Organisation (ECCO) and American Gastroenterological Association (AGA) struggle to deliver the desired remission rates, and many patients must cycle through several different therapies to achieve remission.^{3,4} Following a step-up approach, the first line treatment of mild to moderate left-sided UC or pancolitis is 5-ASA (combined topical and oral administration) for the induction of remission. For refractory patients and in severe disease cases, systemic corticosteroids, azathioprine, 6-mercaptopurine, monoclonal antibodies (such as infliximab, an anti TNF-α; vedolizumab, an anti α₄β₇ integrin; and Ustekinumab, a IL-12 IL-23 blockade) and Ozanimod (a sphingosine 1-phosphate receptor modulator) are the treatments of choice to obtain remission.⁴⁻⁸ Compared to conventional treatment, biological-based therapies have considerable side effects including systemic toxicity, loss of sustained response to therapy with time that produce symptom flares, opportunistic infections, psoriasis and lupus-like syndrome.⁹⁻¹³

Recently, tofacitinib (TOFA), a small-molecule inhibitor of the enzymes Janus kinase 1 and 3 (JAK3 and JAK1, respectively)¹⁴, was approved by European and US regulators for the oral treatment of UC in patients who had intolerance to biologic drugs. Its oral administration is superior to biologic drugs in maintenance of remission and endoscopic improvement.^{15,16} In steroid-refractory UC, the use of tacrolimus (TAC)—a macrolide that inhibits T-lymphocyte activation—is recommended.¹⁷ However, TOFA and TAC showed dose-dependent adverse effects when administered systemically (e.g. nephrotoxicity, thromboembolic complications, headache, metabolic disorders) in a significant fraction of patients,¹⁸⁻²¹ which may require discontinuation of the treatment in some cases.²¹ Taken together, the side effects of these systemically-administered drugs must be weighed in patient management against potential benefits for UC treatment.

The specific localisation of the disease to the colon encourages the use of topical therapies.²² Indeed, delivery via the rectal route is a safer therapeutic approach which can maximise the drug concentration directly at the site of inflammation with minimal systemic exposure, and it is routinely used as first-line treatment for UC,^{23,24} as seen for 5-ASA: rectal administration of this compound in UC patients is significantly more efficient than oral administration.²⁵⁻³⁰ Moreover, resistant ulcerative proctitis has been managed by topically administered TAC as an ointment^{27,31}, suppositories³², and enemas³⁰.

Although clinical studies have shown that rectal preparations are more effective than oral preparations, these treatments are still very rarely prescribed.³³ This is because efficacy of conventional enema-based formulations is intrinsically limited by their insufficient retention in the colon³⁴ and faecal urgency associated with the administration of large volumes.³⁵ Moreover, the required retention time—at least 20 minutes—together with frequent dosing negatively affects the patient compliance.³⁶

Another major difficulty with the use of topical preparations (e.g. enemas) in body cavities, such as the GI tract, is the fact that such cavities are typically coated in a mucous membrane which is non-adherent and turned over rapidly. Thick, viscous preparations can be difficult to apply effectively rectally to the lower GI tract, and are also difficult to manufacture due to high viscosity preventing sterile filtration. Existing compositions are typically either low viscosity and short-lived or longer lived at the price of high viscosity. Furthermore, existing topical compositions are often capable of containing only a low level of active agent, due to poor compatibility between the base composition (e.g. carrier) and the active agent. This results in a composition which rapidly loses effectiveness as it begins to dissipate from the site of action.

Lipid-based drug delivery systems are designed to address challenges like the solubility and bioavailability of poorly water-soluble drugs. Lipid-based formulations can be tailored to meet a wide range of product requirements dictated by disease indication, route of administration, cost consideration, product stability, toxicity, and efficacy. These formulations are also a commercially viable strategy to formulate pharmaceuticals, for topical, oral, pulmonary, or parenteral delivery.

The use of non-lamellar phase structures (such as liquid crystalline phases) in the delivery of bioactive agents is relatively well-known. Such structures form when an amphiphilic compound is exposed to a solvent because the amphiphile has both polar and non-polar groups which cluster to form polar and non-polar regions. These regions can effectively solubilise both polar and non-polar compounds. Such non-lamellar phase formulations are capable of forming liquid crystalline phase structures upon contact with aqueous fluid. However, considering the peculiarity of the rectal milieu, characterised by a low volume and with a composition highly affected by age, biological sex and pathology, aqueous fluid is not the most suitable trigger for an in situ gelation. Furthermore, such non-lamellar systems can exhibit a burst release of the encapsulated active upon exposure to aqueous fluid.

Thus, there remains a need for an improved or alternative delivery system, for use in the treatment of UC. In particular, topical formulations which are bioadherent (i.e. to mucosal surfaces), and which can be formulated as a low viscosity composition which would become adherent upon exposure to a suitable trigger in situ.

Upon hydration, monoacylglycerol lipids (such as monolinolein—MLO, generally recognised as safe for human and/or animal use-GRAS, by the FDA) can self-assemble in different arrangements. By increasing the water content, the less viscous lamellar (L) phase transforms first to an Ia3d and then to a Pn3m cubic phase (Q) which are similar in appearance and rheology to a high viscous cross-linked hydrogel.³⁷ To overcome the hurdle of administering a highly viscous gel, not only water but also temperature can be used as a trigger to tune the viscosity of the system. Thus, as described herein, the inventors have developed a gel platform that employs rectal temperature as a trigger for the formation of a highly viscous adhesive depot system. It has unexpectedly been found that the compositions of the invention can be used to effectively deliver a high concentration of drugs locally to the colonic mucosa, resulting in sustained drug release.

### BRIEF SUMMARY OF THE DISCLOSURE

In accordance with the present invention, there is provided a composition comprising:
a) a carrier comprising:
   a1) water in an amount of more than 10% to 30% by weight of the carrier; and
   a2) lipid in an amount of 70% to 90% by weight of the carrier; and
b) a pharmaceutically active agent,
wherein the lipid is selected from monolinolein or monoolein, and
wherein the composition forms a lipid cubic phase at a temperature of 36° C. to 39° C.

Suitably, the carrier comprises more than 10% to 30% water, and 70% to 90% lipid, wherein the % is % by weight based on the weight of carrier. Thus, in embodiments, the carrier consists of more than 10% to 30% water, and 70% to 90% lipid, wherein the % is % by weight based on the weight of carrier.

Suitably, the lipid is monolinolein.

In embodiments, the composition comprises:
a) a carrier comprising:
   a1) water in an amount of more than 10% to 25% by weight of the carrier; and
   a2) monolinolein in an amount of 75% to 90% by weight of the carrier; and
b) a pharmaceutically active agent,
wherein the composition forms a lipid cubic phase at a temperature of 36° C. to 39° C.

In embodiments the carrier comprises about 16% by weight water and about 84% by weight monolinolein, wherein the % is % by weight of the carrier. In embodiments the carrier comprises 16% by weight water and 84% by weight monolinolein, wherein the % is % by weight of the carrier. In embodiments the carrier consists of about 16% by weight water and about 84% by weight monolinolein, wherein the % is % by weight of the carrier. In embodiments the carrier consists of 16% by weight water and 84% by weight monolinolein, wherein the % is % by weight of the carrier.

In embodiments, the composition of the invention has a lamellar phase structure at 25° C. Thus, in embodiments, the composition is a lamellar gel at 25° C. As illustrated in the Examples herein, temperature can be employed as a trigger factor to convert the lamellar phase structure into a lipid cubic phase. In particular, the inventors have found that rectal temperature can be employed as a trigger factor to convert the lamellar phase structure into a lipid cubic phase, such that when administered in the rectum, the compositions of the invention convert into a lipid cubic phase and act as a highly viscous bioadhesive controlled depot system. Therefore, in embodiments, the composition of the invention forms a lipid cubic phase at a temperature of 36° C. to 39° C. Preferably, the composition of the invention forms a lipid cubic phase at a temperature of 38° C.

In embodiments, the composition of the invention is substantially free from organic solvents.

Also provided, is a composition of the invention, for use as a medicament.

Also provided, is a composition of the invention, for use in treating a condition of the lower gastrointestinal tract.

Also provided, is a method of treating a condition of the lower gastrointestinal tract in a subject, the method comprising administering to the subject an effective amount of a composition of the invention.

Also provided is the use of a composition of the invention, for the manufacture of a medicament for the treatment of a condition of the lower gastrointestinal tract in a subject.

Also provided, is a composition of the invention, for use in the treatment of a condition affecting the colon, wherein the composition is topically applied to the colon and/or the rectum of a subject. Suitably, the composition is administered to the subject via the rectum, wherein the composition coats the lining of the colon and/or the rectum. Thus, the composition is topically applied to the lining of the colon and/or the rectum.

In any of the embodiments herein relating to the composition for use, methods of treatment or use of the composition, it may be that the composition of the invention is administered topically to the subject. Thus, it may be that the composition is topically applied to the colon of the subject. It may be that the composition is topically applied to the rectum of the subject. In some embodiments, the composition is administered to the subject via the rectum. It may be that the composition is administered to the subject via the rectum in the form of: a suppository; a rectal capsule; a semi-solid rectal preparation; a rectal foam; a rectal tampon; or an enema. It may be that the composition is a semi-solid rectal preparation. It may be that the composition is an enema composition.

In embodiments, the composition of the invention has a lamellar phase structure at 25° C., and is administered to the subject via the rectum (e.g. as an enema). Once administered into the rectum (e.g. as an enema), the composition forms a lipid cubic phase at a temperature of 36° C. to 39° C. Preferably, once administered into the rectum (e.g. as an enema), the composition forms a lipid cubic phase at a temperature of 38° C.

In other embodiments herein relating to the composition for use, methods of treatment or use of the composition, it may be that the composition of the invention is an injectable formulation. Thus, it may be that the injectable formulation is a subcutaneous, intramuscular, or intradermal injectable formulation. In some embodiments, the composition is administered to the subject subcutaneously, intramuscularly or intradermally. Preferably, the injectable formulation is a subcutaneous injectable formulation, and thus the composition is administered to the subject subcutaneously.

Also provided, is the use of a formulation comprising more than 10% w/w to 30% w/w water, and 70% w/w to 90% w/w lipid, as a carrier for a pharmaceutically active agent, wherein the lipid is selected from monolinolein or monoolein. Suitably, the lipid is monolinolein. It may be that the formulation comprises more than 10% w/w to 25% w/w water, and 75% w/w to 90% w/w lipid, as a carrier for a pharmaceutically active agent, wherein the lipid is selected from monolinolein or monoolein.

In embodiments the formulation comprises about 16% by weight water and about 84% by weight monolinolein. In embodiments the formulation comprises 16% by weight water and 84% by weight monolinolein. In embodiments the formulation consists of about 16% by weight water and about 84% by weight monolinolein. In embodiments the formulation consists of 16% by weight water and 84% by weight monolinolein.

Also provided, is the use of a pre-formulation composition comprising a lipid and a pharmaceutically active agent for the manufacture of a composition of the invention, wherein the lipid is selected from monolinolein or monoolein. Suitably, the lipid is monolinolein.

Also provided, is a method of making a composition of the invention, comprising:
a) hydrating a mixture comprising a lipid and a pharmaceutically active agent with water, to provide a lipid-drug mixture; and
b) equilibrating the lipid-drug mixture to provide the composition,
   wherein the lipid is selected from monolinolein or monoolein. Suitably, the lipid is monolinolein.

Also provided, is a method of making a composition of the invention, comprising:
a) dissolving a pharmaceutically active agent in water to provide a drug mixture;
b) hydrating a lipid with the drug mixture, to provide a lipid-drug mixture; and
c) equilibrating the lipid-drug mixture to provide the composition,
   wherein the lipid is selected from monolinolein or monoolein. Suitably, the lipid is monolinolein.

Preferably, the pharmaceutically active agent is a hydrophilic pharmaceutically active agent.

Also provided, is a kit comprising:
a) a first container comprising a lipid and a pharmaceutically active agent; and
b) instructions to combine a) with water to provide the composition of the invention,
   wherein the lipid is selected from monolinolein or monoolein. Suitably, the lipid is monolinolein.

Also provided, is a kit comprising:
a) a first container comprising a lipid; and
b) instructions to combine a) with a solution comprising a pharmaceutically active agent dissolved in water to provide the composition of the invention,
   wherein the lipid is selected from monolinolein or monoolein. Suitably, the lipid is monolinolein.

Preferably, the pharmaceutically active agent is a hydrophilic pharmaceutically active agent.

Also provided, is a composition for use in treating a condition of the lower gastrointestinal tract, wherein the composition comprises:
a) a carrier comprising:
   a1) water in an amount of more than 10% to 30% by weight of the carrier; and
   a2) monoacylglycerol lipid in an amount of 70% to 90% by weight of the carrier, wherein the monoacylglycerol lipid comprises monolinolein or monoolein, or combinations thereof; and
b) a pharmaceutically active agent,
wherein the composition forms a lipid cubic phase at a temperature of 36° C. to 39° C., and
wherein the composition is administered rectally;
   preferably wherein the monoacylglycerol lipid is monolinolein.

Also provided, is a composition comprising:
a) a carrier comprising:
   a1) water in an amount of from 14% to 18% by weight of the carrier; and
   a2) monoacylglycerol lipid in an amount of 82% to 86% by weight of the carrier, wherein the monoacylglycerol lipid comprises at least 50% by weight monolinolein; and
b) a pharmaceutically active agent in an amount of 0.1% to 10% by weight of the composition, wherein the composition forms a lipid cubic phase at a temperature of 36° C. to 39° C.

Also provided, is a use of a pre-formulation composition comprising the monoacylglycerol lipid, and the pharmaceutically active agent for the manufacture of a composition as defined herein,
optionally wherein the pre-formulation composition is a lyophilised mixture.

It may be that the pharmaceutically active agent is any of the pharmaceutically active agents as described herein. It may be that the monoacylglycerol lipid comprises at least 50% by weight monolinolein, as described herein. It may be that the monoacylglycerol lipid comprises monolinolein or monoolein, or combinations thereof, as described herein.

Also provided, is a method of making a composition for use as defined herein, or a composition as defined herein, comprising:
a) hydrating a mixture comprising the lipid and the pharmaceutically active agent with water, to provide a lipid-drug mixture; and
b) equilibrating the lipid-drug mixture to provide the composition, optionally wherein:
   A1) the mixture in step a) is a lyophilised mixture; and/or
   A2) the lyophilised mixture is obtained by:
      i) dissolving the lipid and the pharmaceutically active agent in an organic solvent; and
      ii) lyophilising the mixture of i) to provide the lyophilised mixture; and/or
   A3) in step i), the organic solvent is selected from ethanol or methanol, preferably wherein the organic solvent is ethanol.

It may be that the lipid is a monoacylglycerol lipid comprising at least 50% by weight monolinolein, as described herein. It may be that the lipid is a monoacylglycerol lipid comprising monolinolein or monoolein, or combinations thereof, as described herein. It may be that the lipid is selected from monolinolein or monoolein. It may be that the pharmaceutically active agent is any of the pharmaceutically active agents as described herein.

Also provided, is a method of making a composition for use as defined herein, or a composition as defined herein, comprising:
a) dissolving the pharmaceutically active agent in water to provide a drug mixture;
b) hydrating the lipid with the drug mixture, to provide a lipid-drug mixture; and
c) equilibrating the lipid-drug mixture to provide the composition,
optionally wherein the pharmaceutically active agent is a hydrophilic pharmaceutically active agent.

It may be that the lipid is a monoacylglycerol lipid comprising at least 50% by weight monolinolein, as described herein. It may be that the lipid is a monoacylglycerol lipid comprising monolinolein or monoolein, or combinations thereof, as described herein. It may be that the lipid is selected from monolinolein or monoolein. It may be that the pharmaceutically active agent is any of the pharmaceutically active agents as described herein.

Also provided, is a method of making a composition for use as defined herein, or a composition as defined herein, comprising:
a) heating the lipid to provide a molten lipid;
b) mixing the molten lipid with the pharmaceutically active agent, to provide a lipid-drug mixture;
c) mixing the lipid-drug mixture with water; and
d) equilibrating the lipid-drug mixture and water to provide the composition, optionally wherein:
   C1) the molten lipid and the pharmaceutically active agent in step b) is mixed at a temperature of about 30° C. to 70° C., preferably about 40° C. to 60° C.; and/or
   C2) the lipid-drug mixture in step c) is mixed with water in a dual-syringe.

It may be that the lipid is a monoacylglycerol lipid comprising at least 50% by weight monolinolein, as described herein. It may be that the lipid is a monoacylglycerol lipid comprising monolinolein or monoolein, or combinations thereof, as described herein. It may be that the lipid is selected from monolinolein or monoolein. It may be that the pharmaceutically active agent is any of the pharmaceutically active agents as described herein.

Also provided, is a kit comprising:
a) a first container comprising a lipid and a pharmaceutically active agent; and
b) instructions to combine a) with water to provide the composition for use as defined herein, or the composition as defined herein,
   optionally wherein the kit further comprises a second container, wherein the second container comprises water,
   further optionally wherein the lipid and the pharmaceutically active agent in the first container are provided as a lyophilised mixture.

It may be that the lipid is a monoacylglycerol lipid comprising at least 50% by weight monolinolein, as described herein. It may be that the lipid is a monoacylglycerol lipid comprising monolinolein or monoolein, or combinations thereof, as described herein. It may be that the lipid is selected from monolinolein or monoolein. It may be that the pharmaceutically active agent is any of the pharmaceutically active agents as described herein.

Also provided, is a kit comprising:
a) a first container comprising a lipid; and
b) instructions to combine a) with a solution comprising a pharmaceutically active agent dissolved in water to provide the composition for use as defined herein, or the composition as defined herein,
   optionally wherein the kit further comprising a second container, wherein the second container comprises the pharmaceutically active agent dissolved in water,
   further optionally wherein the pharmaceutically active agent is a hydrophilic pharmaceutically active agent.

It may be that the lipid is a monoacylglycerol lipid comprising at least 50% by weight monolinolein, as described herein. It may be that the lipid is a monoacylglycerol lipid comprising monolinolein or monoolein, or combinations thereof, as described herein. It may be that the lipid is selected from monolinolein or monoolein. It may be that the pharmaceutically active agent is any of the pharmaceutically active agents as described herein.

Further aspects and features of the invention are set out in the detailed description below.

### BRIEF DESCRIPTION OF THE DRAWINGS

FIG. 1—SAXS spectra acquired at different temperatures on gels containing 10% w/w of TOFA (FIG. 1A) and 10% w/w of TAC (FIG. 1B).

FIG. 2—In vitro and ex vivo characterisations of the TIF-Gel: FIG. 2A) schematic depiction of the in vitro characterisation and the mechanism of the gel formation. FIG. 2B) SAXS spectra acquired at different temperatures (25° C., 30° C., and 38° C.; bottom, middle and top spectra, respectively) on gels containing increasing amount of water (12%, 14%, 16% and 18% w/w). FIG. 2C) the obtained partial phase diagram (grey circles: L; black circles: coexistence of Ia3d+L; grey squares: Ia3d). FIG. 2D) SAXS spectra acquired at different times (5, 10, 20 and 30 min) after incubation at 38° C. FIG. 2E) frequency sweep at the end of the release experiments (grey circles) and at the beginning of the experiment (grey triangles). FIG. 2F) SAXS before and after the release experiments (1: before; 2: after release in HEPES; 3: after release with lipase). FIG. 2G) Flow and yield points obtained for lamellar phase (light grey bars) and for cubic gel (dark grey bars) from the amplitude sweep experiments. FIG. 2H) In vitro characterisations of the TIF-Gel: SAXS spectra acquired at different temperatures: at 25° C. (bottom), after 30 minutes equilibration at 38° C. (middle) and after 30 minutes equilibration at 25° C. (top). SAXS was used to determine the lipid phase, and thus the reversibility of the transition. FIG. 2I) Amplitude sweep experiments acquired at 25° C. (triangle symbols) and at 38° C. (circle symbols) on empty gel. a) Storage moduli (G′) and loss of moduli (G″) are plotted versus the shear stress. The yield point is the value of the shear stress at the limit of the LVE region while the flow point is the value of the shear stress at the crossover point G′=G″. b) Shear strain is plotted versus the stress, and the yield point is exceeded at the point where the deformations start to deviate from linearity. FIG. 2J) SAXS spectra acquired at different time points (before administration, excreted with the stool, and the residual gel present in the colon) at 38° C.

FIG. 3—In vitro and ex vivo characterisations of the TIF-Gel: FIG. 3A) TOFA-loaded gel (TIF-Gel-TOFA) SAXS spectra acquired at different temperatures. FIG. 3B) in vitro release of free drug (TOFA, grey squares) and TIF-Gel-TOFA in HEPES buffer (TIF-Gel-TOFA, black triangles) and in the presence of lipase (TIF-Gel-TOFA (lipase), grey triangles) (results are reported as mean±STDV (n=3)). FIG. 3C) ex vivo release of free drug (TOFA, grey squares) and TIF-Gel-TOFA (TIF-Gel-TOFA, black triangles) (results are reported as mean±STDV (n=3)). FIG. 3D) TAC loaded LMPS (TIF-Gel-TAC) SAXS spectra acquired at different temperatures. FIG. 3E) in vitro release of free drug (TAC, grey squares), TIF-Gel-TAC (TIF-Gel-TAC, black triangles) and in the presence of lipase (TIF-Gel-TAC (lipase), grey triangles) (results are reported as mean±STDV (n=3)). FIG. 3F) ex vivo release of free drug (TAC, grey squares) and TIF-Gel-TAC in HEPES buffer (TIF-Gel-TAC, black triangles) (results are reported as mean±STDV (n=3)). FIG. 3G) schematic depicting in vitro release experiments, wherein drug-loaded TIF-Gel formulations were placed in the donor chamber of a vertical diffusion cell. FIG. 3H) Drugs distribution into TIF-Gel. a) WAXS spectra obtained for empty gel (bottom), TOFA loaded gel (middle) and TAC loaded-gel (top). All the WAXS spectra (acquired for 30 minutes at 25° C.) show only a broad shoulder (and no evident peak) which indicates the amorphous state of the lipidic chain and the absence of crystalline structures. b) Homogeneity of drugs in the gel. The amount of drug present in 3 different gel layers (Top, Middle and Bottom) was evaluated by HPLC.

FIG. 4—In vitro release of free drug and TIF-Gel-loaded drug in HEPES buffer, over a period of 8 h. FIG. 4A) In vitro release of clotrimazole (‘Free drug’, square symbols) and TIF-Gel-loaded clotrimazole (‘LC’, circle symbols) in HEPES buffer. FIG. 4B) In vitro release of mesalamine (‘Free drug’, circle symbols) and TIF-Gel-loaded mesalamine (‘LC’, square symbols) in HEPES buffer. FIG. 4C) In vitro release of budesonide (‘Free drug’, square symbols) and TIF-Gel-loaded budesonide (‘LC’, circle symbols) in HEPES buffer.

FIG. 5—TIF-Gel-TOFA effectively mitigates intestinal inflammation and disease induced by DSS treatment in mice. Mice were prophylactically treated rectally with either empty gel (TIF-Gel; n=6), tofacitinib in vehicle (TOFA; n=7), or TOFA loaded-gel (TIF-Gel-TOFA; n=6) and thereafter challenged with 2% DSS in the drinking water. Treatments were then applied every other day until the end of the experiment. Weights (FIG. 5A) and disease score (FIG. 5B) were recorded throughout the experiment. At the end of the experiment, spleens, mesenteric lymph nodes (mLNs) and colons were removed from the mice. The spleens were weighed (FIG. 5C) and single splenocytes were enumerated (FIG. 5D). The tissue concentrations of various cytokines were measured (FIG. 5E). The mouse colon length was measured (FIG. 5F), and the colon was opened transversally, cleaned, and prepared for histology (FIG. 5G). Colon histopathology scores were determined and aggregated (FIG. 5H). *: p<0.05, **: p<0.01, ***: p<0.001 and actual value is provided for values less than 0.1 but not meeting significance threshold as determined by 2-way ANOVA (FIG. 5A), multiple Student's-tests with Holm-Sidak correction for multiple comparisons (FIGS. 5B and 5E), and one way ANOVA with multiple comparisons and Tukey correction (FIGS. 5C, 5D, 5F, 5H). All tests were performed using Prism (GraphPad) and applying default settings for the above-mentioned analyses; all error bars are ±SEM.

FIG. 6—Assessment of the effect of TAC-loaded gel on T cell-mediated colitis: 12-15-week-old Rag^{-/-} mice develop colitis via transfer of 2.5×10⁵ naïve T cells. Starting on day 3 post T cell transfer, mice received daily rectal instillations with TIF-Gel without drug (TIF-Gel), TAC-loaded TIF-Gels (TIF-Gel-TAC) or TAC in vehicle (TAC). FIG. 6A) Schematic overview on the experimental set-up. FIG. 6B) weight development over the course of the experiment. FIG. 6C) cumulative disease activity score. FIGS. 6D and 6E) representative pictures and respective scoring from mouse colonoscopy on day 19 post T cell transfer and from H&E-stained sections of the terminal colon collected on day 19 post T cell transfer.

FIG. 7—Immune cell populations in the colon from TAC-loaded TIC-Gel treated mice: 12-15-week-old Rag^{-/-} mice develop colitis via transfer of 2.5×10⁵ naïve T cells. Starting on day 3 post T cell transfer, mice received daily rectal instillations with TIF-Gel without drug (TIF-Gel), TAC-loaded TIF-Gels (TIF-Gel-TAC) or TAC in vehicle (TAC). Depicted are the relative abundance of the indicated cell populations in FIG. 7A) the colonic lamina propria, FIG. 7B) mesenteric lymph nodes; and FIG. 7C) the spleen on day 19 after T cell transfer, and (d) levels of indicated cytokines in colon lysates. *p<0.05, **p<0.01, ***p<0.001 as determined by one way ANOVA with multiple comparisons and Tukey correction. All tests were performed using Prism (GraphPad) and applying default settings for the above-mentioned analyses; all error bars are ±SEM.

FIG. 8—Long-term stability of TOFA loaded into TIF-gel (FIG. 8A) and TAC loaded into TIF-gel (FIG. 8B) over one month, as analysed by HPLC. Samples were stored at 4° C. (black bars) and 25° C. (grey bars) over the course of the study. Data are expressed as percentage±SD.

FIG. 9—Drug delivery via TIF-Gel leads to a low systemic drug exposure. (a) Experimental design for the pharmacokinetic study. Healthy mice (n=5/group) received a single enema of either drug-loaded TIF-Gel (TIF-Gel-TOFA or TIF-Gel-TAC) or free drugs (TOFA or TAC). The plasma drug concentrations were measured at the indicated time points after administration. Plasma concentration versus time profiles of the pharmacokinetic experiment of TOFA- (b) and TAC-treated animals (c) and Area Under the Curve (AUC)_{0-48 h} values of TOFA- and TAC-treated mice (d and e, respectively). ***p<0.001, as determined by Student's t test.

FIG. 10—The TIF-Gel adheres to healthy colonic tissue for at least 6 hours. a) Experimental scheme depicting the procedure: Healthy animals received an enema of 100 μL of DIR-TIF-Gel. Animals were sacrificed after 30 min, 2 and 6 h and the colon was harvested and imaged (b). c) The obtained signal was analysed as radiant efficiency (RE), which was normalised to radiant efficacy recorded at 30 min.

### DETAILED DESCRIPTION

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

For the avoidance of doubt, the information disclosed earlier in this specification under the heading “Background” is relevant to the invention and is to be read as part of the disclosure of the invention.

The reader's attention is directed to all papers and documents which are filed concurrently with or previous to this specification in connection with this application and which are open to public inspection with this specification, and the contents of all such papers and documents are incorporated herein by reference.

### Definitions

Unless otherwise stated, the following terms used in the specification and claims have the following meanings set out below.

The term “treatment”, and the therapies encompassed by this invention, include the following and combinations thereof: (1) reducing the risk of or inhibiting, e.g. delaying, initiation and/or progression of, a state, disorder or condition; (2) preventing, e.g. reducing the risk of, or delaying the appearance of clinical symptoms of a state, disorder or condition developing in a patient (e.g. human or animal) that may be afflicted with or predisposed to the state, disorder or condition but does not yet experience or display clinical or subclinical symptoms of the state, disorder or condition; (3) inhibiting the state, disorder or condition (e.g., arresting, reducing or delaying the development of the disease, or a relapse thereof in case of maintenance treatment, of at least one clinical or subclinical symptom thereof); and/or (4) relieving the condition (e.g. causing regression of the state, disorder or condition or at least one of its clinical or subclinical symptoms). Where the composition of the invention is used in the treatment of a patient, treatment contemplates any one or more of: maintaining the health of the patient; restoring or improving the health of the patient; and delaying the progression of the disorder. The benefit to a patient to be treated may be either statistically significant or at least perceptible to the patient or to the physician. It will be understood that a medicament will not necessarily produce a clinical effect in every patient to whom it is administered, and this paragraph is to be understood accordingly. The compositions and methods described herein are of use for therapy and/or prophylaxis of disease. The compositions and methods described herein are of use for inhibiting or preventing disease progression.

The treatments may include maintenance therapy of patients who have suffered a disorder and whose condition has subsequently improved, e.g. because of treatment. Such patients may or may not suffer a symptomatic disorder. Maintenance therapy aims to arrest, reduce or delay (re-)occurrence or progression of a disorder.

Reference herein to a “therapeutically effective amount” is an amount sufficient to reduce or completely alleviate symptoms or other detrimental effects of a disorder; reverse, completely stop, or slow the progress of a disorder; or reduce the risk of a disorder getting worse; for example, an amount sufficient to induce remission of ulcerative colitis, or an amount sufficient to maintain a remission of ulcerative colitis. It is further within the skill of one of ordinary skill in the art to determine appropriate treatment duration, appropriate doses, and any potential combination treatments, based upon an evaluation of therapeutic or prophylactic response.

Reference to “modified release” herein includes compositions which alter the release of a drug from the composition, particularly compositions which for example provide controlled release, extended (or sustained) release or delayed release or any combination thereof, for example delayed and controlled release of a drug from a composition following administration, e.g. following rectal administration via enema.

The term “Cₘ-Cₙ” refers to a group with m to n carbon atoms.

The term “C₁-C₆-alkyl” refers to a linear or branched hydrocarbon chain containing 1, 2, 3, 4, 5 or 6 carbon atoms, for example methyl, ethyl, n-propyl, iso-propyl, n-butyl, sec-butyl, tert-butyl, n-pentyl and n-hexyl. “C₆-C₃₂-alkyl” similarly refers to such groups containing 6 carbon atoms up to 32 carbon atoms. The alkyl groups may be unsubstituted or substituted by one or more substituents. Substituents for the alkyl group may be halogen, e.g. fluorine, chlorine, bromine and iodine, OH, C₁-C₄ alkoxy.

The term “antibody”, in the context of the present invention, refers to “immunoglobulin” (Ig), which is defined as a protein belonging to the class IgG, IgM, IgE, IgA, or IgD (or any subclass thereof), and includes all conventionally known antibodies and functional fragments thereof. In the context of the present invention, a “functional fragment” of an antibody/immunoglobulin is defined as antigen-binding fragment or other derivative of a parental antibody that essentially maintains the properties of such a parental antibody. An “antigen-binding fragment” of an antibody/immunoglobulin is defined as a fragment (e.g., a variable region of an IgG) that retains the antigen-binding region. An “antigen-binding region” of an antibody typically is found in one or more hypervariable region(s) of an antibody, i.e., the CDR-1, -2, and/or -3 regions. “Antigen-binding fragments” according to the invention include the domain of a F(ab′)2 fragment and a Fab fragment. “Functional fragments” of the invention include Fab fragment, F(ab′)2 fragment, Fab′ fragment, scFv, dsFv, VHH, diabody, triabody, tetrabody, Fc fusion protein and minibody. The F(ab′)2 or Fab domain may be engineered to minimise or completely remove the intermolecular disulphide interactions that occur between the CH1 and CL domains. The antibodies or functional fragments used for the present invention may be part of bi- or multifunctional constructs.

The term “immunosuppressive agent” is intended to mean pharmacologically acceptable compounds that have the effect of suppressing immune response in the human or animal body. The term “antineoplastic agent” is intended to mean pharmacologically acceptable compounds that are cytotoxic to neoplastic cells.

The term “gel” is used herein refers to a semi-solid, apparently homogeneous substance that may be elastic and jelly-like (as in gelatin). The gel comprises a three-dimensional polymeric or inorganic matrix within which is dispersed a liquid phase. The matrix of the gel comprises a network of physically or chemical cross-linked polymers or copolymers that swell but do not dissolve in the presence of a solvent. The cross-linking within the gel matrix may be physical cross linking (for example by hydrogen bonding or ionic cross-linking) or may be covalently cross-linked. The gels are generally clear in appearance, however, turbid gels are also contemplated. The U.S.P. defines gels as a semi-solid system consisting of dispersion made up of either small inorganic particles or large organic molecule enclosing and interpenetrated by liquid. The European Pharmacopoeia defines gels as semi-solid preparations consisting of a single-phase liquid basis gelled by a suitable gelling agent. Active substance(s) are dissolved or dispersed in the basis.

In embodiments, the carrier of the composition comprises: a1) water in an amount of more than 10% to 30% by weight of the carrier; and a2) lipid in an amount of 70% to 90% by weight of the carrier. It may be that the lipid is selected from monolinolein or monoolein. In embodiments, the carrier of the composition consists of a1) water in an amount of more than 10% to 30% by weight of the carrier; and a2) lipid in an amount of 70% to 90% by weight of the carrier, wherein the lipid is selected from monolinolein or monoolein. It is to be understood that references herein to the embodiments which refer to “a carrier comprising” the lipid and water also encompasses embodiments wherein the carrier consists only of the lipid and water. Thus by way of example it may be that the carrier consists only of monolinolein and water in the amounts specified in any of the embodiments herein.

The composition also comprises a pharmaceutically active agent, for example from 0.1% to 10% of the pharmaceutically active agent, wherein the % is % by weight based on the weight of the composition. Thus, the carrier acts as a vehicle for the pharmaceutically active agent within the composition. It would be clear to the skilled person that reference to the term ‘carrier’ relates to the lipid and water component of the total composition. For example, it may be that the composition comprises: a) a carrier comprising: a1) water in an amount of 10% by weight of the carrier; and a2) lipid in an amount of 90% by weight of the carrier; and b) a pharmaceutically active agent in an amount of 10% by weight of the composition. In this instance, the total composition comprises:
a) 90% of a carrier (wherein the % is % by weight of the composition), wherein the carrier comprises:
   a1) water in an amount of 10% by weight of the carrier; and
   a2) lipid in an amount of 90% by weight of the carrier; and
b) 10% of a pharmaceutically active agent (wherein the % is % by weight of the composition).

In each of the examples listed above, the ratio of the components in the carrier are fixed (i.e. 10% water and 90% lipid), whereas the amount of the carrier in the total composition varies according to the amount of pharmaceutically active agent present in the composition.

The term “carrier” refers to a formulation comprising, or preferably consisting, the water and the lipid (e.g. monolinolein or monoolein). The “composition” comprises the carrier, the pharmaceutically active agent, and optionally other components (e.g. additive(s)). The term “by weight of the carrier” refers to a total weight of the water and the lipid (e.g. monolinolein or monoolein) present in the carrier. Thus, it may be that the composition of the invention comprises:
a) a carrier comprising:
   a1) more than 10% to 30% by weight of water;
   a2) 70% to 90% by weight of lipid; and
b) 0.1% to 10% by weight of the composition of a pharmaceutically acceptable agent,
   wherein the total weight of a1) to a2) is 100%, and the term “by weight” of a1) and a2) refers to “% by weight of the carrier”. In this example the % by weight of the pharmaceutically acceptable agent refers to the % by weight of the total weight of the composition containing all components of the composition.

References herein to “the pharmaceutically acceptable agent is present in an amount of about x % by weight of the carrier” refer to the weight percentage of the pharmaceutically acceptable agent as compared to the total weight of the carrier comprising the water and the lipid (e.g. monolinolein or monoolein).

The skilled person will be aware that a composition of the invention comprising: a) 90-99.9% of a carrier (wherein the % is % by weight of the composition), wherein the carrier comprises: a1) water in an amount of 10-30% by weight of the carrier; and a2) lipid in an amount of 70-90% by weight of the carrier; and b) a pharmaceutically active agent in an amount of 0.1-10% of by weight of the composition, is equivalent to a composition comprising:
(i) 0.1% w/w to 10% w/w of a pharmaceutically active agent;
(ii) 63% w/w to 89.9% w/w lipid; and
(iii) 9% w/w to 29.97% w/w water,
wherein the % is % by weight of the composition,
wherein the lipid is selected from monolinolein or monoolein,
wherein the composition forms a lipid cubic phase at a temperature of 36° C. to 39° C.

The skilled person will also be aware that a composition of the invention comprising: a) 90-99.9% of a carrier (wherein the % is % by weight of the composition), wherein the carrier comprises: a1) water in an amount of 10-25% by weight of the carrier; and a2) lipid in an amount of 75-90% by weight of the carrier; and b) a pharmaceutically active agent in an amount of 0.1-10% of by weight of the composition, is equivalent to a composition comprising:
(i) 0.1% w/w to 10% w/w of a pharmaceutically active agent;
(ii) 67.5% w/w to 89.9% w/w lipid; and
(iii) 9% w/w to 24.9% w/w water,
wherein the % is % by weight of the composition,
wherein the lipid is selected from monolinolein or monoolein,
wherein the composition forms a lipid cubic phase at a temperature of 36° C. to 39° C. For example, it may be that the composition comprises: a) a carrier comprising: a1) water in an amount of 10% by weight of the carrier; and a2) lipid in an amount of 90% by weight of the carrier; and b) a pharmaceutically active agent in an amount of 5% by weight of the composition. In this instance, the total composition comprises:
(i) 5% w/w of a pharmaceutically active agent;
(ii) 85.5% w/w lipid; and
(iii) 9.5% w/w water.

In another example, it may be that the composition comprises: a) a carrier comprising: a1) water in an amount of 10% by weight of the carrier; and a2) lipid in an amount of 90% by weight of the carrier; and b) a pharmaceutically active agent in an amount of 1% by weight of the composition. In this instance, the total composition comprises:
(i) 1% w/w of a pharmaceutically active agent;
(ii) 89.1% w/w lipid; and
(iii) 9.9% w/w water.

For the avoidance of doubt, monolinolein (2,3-dihydroxypropyl(9Z,12Z)-9,12-octadecadienoate) has the following structure:

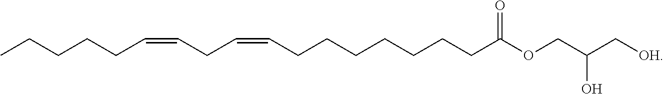 [SMILES: CCCCC/C=C\C/C=C\CCCCCCCC(=O)OCC(O)CO]

For the avoidance of doubt, monoolein (2,3-dihydroxypropyl(9Z)-9-octadecenoate) has the following structure:

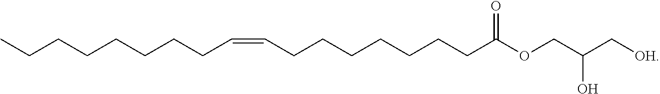 [SMILES: CCCCCCCC/C=C\CCCCCCCC(=O)OCC(O)CO]

The term “rectal temperature” refers to a temperature range of from about 36° C. to about 39° C. Thus, in embodiments described herein, the term rectal temperature may refer to a temperature of 36.0° C., 36.1° C., 36.2° C., 36.3° C., 36.4° C., 36.5° C., 36.6° C., 36.7° C., 36.8° C., 36.9° C., 37.0° C., 37.1° C., 37.2° C., 37.3° C., 37.4° C., 37.5° C., 37.6° C., 37.7° C., 37.8° C., 37.9° C., 38.0° C., 38.1° C., 38.2° C., 38.3° C., 38.4° C., 38.5° C., 38.6° C., 38.7° C., 38.8° C., 38.9° C., or 39.0° C. In particular, it may be the term rectal temperature refers to a temperature of about 38° C. Preferably, the term rectal temperature refers to a temperature of 38° C.

Reference to “rectal administration” or “administered rectally” covers any route of administration (e.g. topical administration) of the composition of the invention via the rectum, directly to the tissues of the lower gastrointestinal (GI) tract. The lower GI tract is commonly divided into three main parts: the colon, the rectum, and the anal canal. The colon is commonly divided into 5 major segments. The right colon comprises the cecum, the ascending colon, the hepatic flexure and the right half of the transverse colon. The left colon comprises the left half of the transverse colon, the descending colon, the splenic flexure and the sigmoid colon. The rectum is the last anatomic segment before the anus. Thus, “rectal administration” or “administered rectally” also covers any route of administration (e.g. topical administration) of the composition of the invention via the rectum, directly to the tissues of the colon (e.g. sigmoid colon, descending colon, transverse colon, ascending colon), rectum, and/or anus. Preferably, the composition of the invention is administered rectally to the rectum, sigmoid colon, and/or the descending colon. More preferably, the composition of the invention is administered rectally to the rectum and/or sigmoid colon. “Rectal administration” or “administered rectally” also covers administration of the composition of the invention via a stoma (e.g. where the subject has had a colostomy).

The compositions (for example the lamellar gel compositions) of the present invention are also referred to as “TIF-Gel” compositions, by which is meant that the composition is a temperature-triggered in situ forming bioadhesive lipid gel. The compositions are triggered at a temperature of from 36° C. to 39° C., preferably 38° C. In preferred embodiments, the compositions are administered to the rectum (e.g. as an enema). Thus, the compositions are triggered by the rectal temperature (as defined herein). The compositions of the present invention undergo a phase transition upon exposure to the temperature trigger, and thus convert from a lamellar phase structure to a lipid cubic phase at a temperature of from 36° C. to 39° C., preferably 38° C. The term, ‘in situ’ may be considered to mean that the composition undergoes this phase transition from a lamellar phase structure to a lipid cubic phase once the composition has been injected into the rectum of a subject, and the composition reaches rectal temperature (as defined herein).

The compositions of the present invention are substantially free from organic solvents. Thus, the compositions disclosed herein contain less than 10%, less than 5%, less than 1%, or suitably less than 0.01%, or preferably less than 0.001% of organic solvents. Preferably the compositions of the invention contain no detectable organic solvents.

In some embodiments, the compositions of the present invention are substantially free from surfactants. In some embodiments the compositions of the present invention are substantially free from non-ionic surfactants, e.g. poloxamers. Thus, in some embodiments, the compositions disclosed herein contain less than 10%, less than 5%, less than 1%, or suitably less than 0.01%, or preferably less than 0.001% of surfactants (e.g. non-ionic surfactants, such as poloxamers). Reference to surfactants in this paragraph does not encompass the lipids present in the carrier of the compositions of the invention.

A “topical formulation” is a formulation that is applied to body surfaces such as the skin or mucous membranes to treat. Topical formulations may also be applied to the surface of tissues other than the skin, for example to the surface of a tooth, rectally or vaginally. Topical formulations differ from many other types of drugs because mishandling them can lead to certain complications in a patient or administrator of the drug. Suitably, the compositions described herein are applied topically to the colon. Preferably, the topical formulations described herein are administered rectally, for example, as an enema.

The terms “lamellar”, “lamellar gel”, “lamellar geometry”, “lamellar phase”, “lamellar phase structure”, “L”, refer to a two-dimensional stack of amphiphilic bilayers separated by aqueous layers. Each bilayer consists of two monolayers packed tail-to-tail to minimise contact between the hydrocarbon chains and water. In this arrangement, water partitions nearly exclusively to the lipid polar heads and forms slabs of water-lipid heads. Accordingly, the composition of the invention is in the lamellar phase at 25° C. as determined by small angle X-ray scattering (SAXS).

The terms “cubic”, “lipid cubic phase”, “cubic geometry”, “lipid cubic phase structure”, “cubic phase”, “Q”, refer to a bicontinuous cubic phase composed of two sets of water channels separated by curved bilayers in the 3D space such that every point on the bilayer midplane surface is a saddle point with a zero-mean curvature. The bilayers encompass a system of aqueous channels and form structured yet flexible networks that are nonbirefringent and optically transparent. Specifically, lipid molecules in bicontinuous cubic phases form a highly curved continuous bilayer that separates the two interpenetrating but nonintersecting aqueous channel networks. The structures can display a double gyroid (Ia3d, with threefold connectivity of aqueous channels), double diamond (Pn3m, with fourfold connectivity), and primitive (Im3m, with sixfold connectivity symmetry). Accordingly, the composition of the invention is in the cubic phase at from about 36° C. to about 39° C., as determined by small angle X-ray scattering (SAXS). In embodiments, it may be that the composition of the invention is in the cubic-Ia3d phase at from about 36° C. to about 39° C. In embodiments, it may be that the composition of the invention is in the cubic-pn3m phase at from about 36° C. to about 39° C. when present in an aqueous environment. For example, in some embodiments the composition of the invention absorbs water at the site of administration to a subject (e.g. following rectal administration) and forms the cubic-pn3m phase at a temperature of from about 36° C. to about 39° C. As discussed above, SAXS may be used to determine the lipid phase, and thus construct the partial phase diagram for different lipid-water systems (e.g. MLO-water systems). The method relies on constructive interferences in the reciprocal space from many ordered scattering planes that belong to the mesophase. An X-ray beam is directed at the lipid sample, and the resulting scattering pattern gives a characteristic set of rings, or maxima, that correspond to Bragg reflections. Their positions in the reciprocal space depend on the Miller indices of the mesophase scattering planes, and the sequence of Bragg reflections (and their ratio) consequently identifies the symmetry of the mesophase studied. SAXS allows for the lattice parameter—the size of the repeat unit cell—to be determined. When the parameter is translated, it is possible to reconstruct the entire mesophase in 3D. SAXS measurements were used to determine the phase identity and symmetry of the produced LMPs. Measurements were performed on a Bruker AXS Micro, with Cu Kα radiation of 1.5418 Å, as described in further detail under ‘Analytical Methods’. The following sequences of Bragg reflections were used to determine the symmetry of each of the mesophases studied: L: 1: 2:3: 4; Ia3d: √6: √8: √14: √16: √20: √22; Pn3m: √2: √3: √4: √6: √8: √9; and H: √1: √3: √4.^{37, 38, 39}

Cross-polarised optical microscopy may also be used to determine the lipid phase.⁴⁰ Lyotropic liquid crystals organise themselves into networks with unique symmetry, which means that their basic motif repeats itself periodically. Liquid crystal cubic phases (Ia3d, pn3m or Im3m) are isotropic, meaning that they do not have any birefringent properties. As such, if a slide with a layer of lyotropic liquid crystal film is placed under a light source that allows polarised light to pass through, it appears black when observed through another polariser tilted at 90°. On the other hand, lamellar phases are non-isotropic (anisotropic), meaning that they have birefringent properties. As such, if a slide with a layer of lamellar gel is placed under a light source that allows polarized light to pass through, it appears coloured when observed through another polariser tilted at 90°.

Reference to a “pharmaceutically acceptable salt” of a compound means a salt that is pharmaceutically acceptable and that possesses the desired pharmacological activity of the parent compound. Such pharmaceutically-acceptable salts may be for example, an acid-addition salt of a compound, for example an acid-addition salt with an inorganic or organic acid such as hydrochloric, hydrobromic, sulfuric, trifluoroacetic, citric or maleic acid; or, for example, a salt of a compound which is sufficiently acidic, for example an alkali or alkaline earth metal salt such as a sodium, calcium or magnesium salt, or an ammonium salt, or a salt with an organic base such as methylamine, dimethylamine, trimethylamine, piperidine or morpholine.

The terms “hydrophobic pharmaceutically active agent”, “hydrophobic pharmaceutically acceptable agent” or “hydrophobic agent” as used herein refers to agents having a greater solubility in organic solvents of low polarity, such as long chain alcohols, than in aqueous solution. “Hydrophobic” means “water-hating” and is used herein to indicate agents that are weakly soluble or insoluble in water and soluble in non-polar solvents.

The terms “hydrophilic pharmaceutically active agent”, “hydrophilic pharmaceutically acceptable agent” or “hydrophilic agent” as used herein refers to agents having a higher solubility in aqueous medium. “Hydrophilic” means “water-loving” and is used herein to indicate agents that are water soluble, i.e. having a strong affinity for water.

Ingredients and excipients of the described compositions are suitable for the intended purpose. For example, pharmaceutical compositions comprise pharmaceutically acceptable ingredients.

If not otherwise stated, ingredients, components, excipients etc. of the compositions of the invention are suitable for one or more of the intended purposes discussed elsewhere herein.

Where the invention is referred to as a formulation it takes the same meaning as the composition of the invention. Accordingly, the terms formulation and composition are used interchangeably.

Where the composition of the invention is referred to as a lamellar gel, it takes the same meaning as the composition of the invention with a lamellar phase structure. Accordingly, these terms are used interchangeably.

Reference to “about” in the context of a numerical is intended to encompass the value+/−10%. For example, about 20% includes the range of from 18% to 22%.

### Composition

Provided herein is a composition comprising:
a) a carrier comprising:
   a1) water in an amount of more than 10% to 30% by weight of the carrier; and
   a2) lipid in an amount of 70% to 90% by weight of the carrier; and
b) a pharmaceutically active agent,
   wherein the composition forms a lipid cubic phase at a temperature of 36° C. to 39° C.

It may be that the composition comprises:
a) a carrier comprising:
   a1) water in an amount of more than 10% to 25% by weight of the carrier; and
   a2) lipid in an amount of 75% to 90% by weight of the carrier; and
b) a pharmaceutically active agent,
   wherein the composition forms a lipid cubic phase at a temperature of 36° C. to 39° C.

Also provided herein, is a composition comprising:
a) a carrier comprising:
   a1) water in an amount of from 14% to 18% by weight of the carrier; and
   a2) monoacylglycerol lipid in an amount of 82% to 86% by weight of the carrier, wherein the monoacylglycerol lipid comprises at least 50% by weight monolinolein; and
b) a pharmaceutically active agent in an amount of 0.1% to 10% by weight of the composition,
wherein the composition forms a lipid cubic phase at a temperature of 36° C. to 39° C.

The lipid is a neutral lipid component comprising a polar “head” group and also non-polar “tail” groups. Generally, the head and tail portions of the lipid will be joined by an ester moiety but this attachment may be by means of an ether, an amide, a carbon-carbon bond or other attachment. Preferred polar head groups are non-ionic, and include polyols such as glycerol, diglycerol and sugar moieties (such as inositol and glucosyl based moieties); and esters of polyols, such as acetate or succinate esters. Preferred polar groups are glycerol and diglycerol, especially glycerol.

Suitably, the lipid is a monoacylglycerol lipid. The non-polar group may be saturated or unsaturated. Examples of non-polar groups include C₆-C₃₂ alkyl and C₆-C₃₂ alkenyl groups, which are typically present as the esters of long chain carboxylic acids. These are often described by reference to the number of carbon atoms and the number of unsaturations in the carbon chain. Thus, CX: Z indicates a hydrocarbon chain having X carbon atoms and Z unsaturations. Examples particularly include caproyl (C6: 0), capryloyl (C8: 0), capryl (C10: 0), lauroyl (C12: 0), myristoyl (C14: 0), palmitoyl (C16: 0), phytanoly (C16: 0), palmitolcoyl (C16: 1), stearoyl (C18: 0), oleoyl (C18: 1), elaidoyl (C18: 1), linoleoyl (C18: 2), linolenoyl (C18: 3), arachidonoyl (C20: 4), behenoyl (C22: 0) and lignoceroyl (C24: 9) groups. Thus, typical non-polar chains are based on the fatty acids of natural ester lipids, including caproic, caprylic, capric, lauric, myristic, palmitic, phytanic, palmitolic, stearic, oleic, elaidic, linoleic, linolenic, arachidonic, behenic or lignoceric acids, or the corresponding alcohols. Preferable non-polar chains are oleic and linoleic acids, particularly linoleic acid. The skilled person would be aware that lipids are naturally derived or semisynthetic raw materials, and thus the source of the raw material and hence its composition can differ. Thus, the skilled person would understand that any reference herein to monolinolein lipids would encompass both pure forms of monolinolein, and commercial-grade forms of monolinolein (e.g. a food-grade form of monolinolein). For example, a commercial-grade form of monolinolein lipid may comprise a mixture of monoacylglycerol lipids (such as monolinolein, and monoolein), and optionally further additional lipids. The skilled person would also understand that any reference herein to monoolein lipids would encompass both pure forms of monoolein, and commercial-grade forms of monoolein (e.g. food-grade forms of monoolein). For example, a commercial-grade form of monoolein lipid may comprise a mixture of monoacylglycerol lipids (such as monoolein, and monolinolein), and optionally further additional lipids.

Thus, it may be that a monolinolein lipid comprises more than about 90 wt % of monoacylglycerol lipids. It may be that a monolinolein lipid comprises more than about 95 wt % of monoacylglycerol lipids. It may be that a monolinolein lipid comprises more than about 98 wt % of monoacylglycerol lipids. It may be that a monolinolein lipid comprises more than about 99.9 wt % of monoacylglycerol lipids. For example, it may be that a monolinolein lipid (e.g. a commercial-grade form of monolinolein lipid) comprises about 90 wt %, 91 wt %, 92 wt %, 93 wt %, 94 wt %, 95 wt %, 96 wt %, 97 wt %, 98 wt %, 99 wt % or 100 wt % of monoacylglycerol lipids.

It may be that a monolinolein lipid comprises up to 100 wt % of monolinolein. It may be that a monolinolein lipid comprises more than or equal to about 99 wt % of monolinolein. It may be that a monolinolein lipid comprises more than about 90 wt % of monolinolein. It may be that a monolinolein lipid comprises more than about 80 wt % of monolinolein. It may be that a monolinolein lipid comprises more than about 70 wt % of monolinolein. It may be that a monolinolein lipid comprises more than about 60 wt % of monolinolein. It may be that a monolinolein lipid comprises more than about 50 wt % of monolinolein. It may be that a monolinolein lipid comprises more than about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 wt % of monolinolein.

It may be that a monolinolein lipid comprises no monoolein. It may be that a monolinolein lipid comprises less than about 50 wt % of monoolein. It may be that a monolinolein lipid comprises less than about 40 wt % of monoolein. It may be that a monolinolein lipid comprises less than about 30 wt % of monoolein. It may be that a monolinolein lipid comprises less than about 20 wt % of monoolein. It may be that a monolinolein lipid comprises less than about 10 wt % of monoolein. It may be that a monolinolein lipid comprises less than about 5 wt % of monoolein. It may be that a monolinolein lipid comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 wt % monoolein.

It may be that a monoolein lipid comprises more than about 90 wt % of monoacylglycerol lipids. It may be that a monoolein lipid comprises more than about 95 wt % of monoacylglycerol lipids. It may be that a monoolein lipid comprises more than about 98 wt % of monoacylglycerol lipids. It may be that a monoolein lipid comprises more than about 99.9 wt % of monoacylglycerol lipids. For example, it may be that a monoolein lipid (e.g. a commercial-grade form of monoolein lipid) comprises about 90 wt %, 91 wt %, 92 wt %, 93 wt %, 94 wt %, 95 wt %, 96 wt %, 97 wt %, 98 wt %, 99 wt % or 100 wt % of monoacylglycerol lipids. 102. It may be that a monoolein lipid comprises up to 100 wt % of monoolein. It may be that a monoolein lipid comprises more than or equal to about 99 wt % of monoolein. It may be that a monoolein lipid comprises more than about 90 wt % of monoolein. It may be that a monoolein lipid comprises more than about 80 wt % of monoolein. It may be that a monoolein lipid comprises more than about 70 wt % of monoolein. It may be that a monoolein lipid comprises more than about 60 wt % of monoolein. It may be that a monoolein lipid comprises more than about 50 wt % of monoolein. It may be that a monoolein lipid comprises more than about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 wt % of monoolein.

It may be that a monoolein lipid comprises no monolinolein. It may be that a monoolein lipid comprises less than about 50 wt % of monolinolein. It may be that a monoolein lipid comprises less than about 40 wt % of monolinolein. It may be that a monoolein lipid comprises less than about 30 wt % of monolinolein. It may be that a monoolein lipid comprises less than about 20 wt % of monolinolein. It may be that a monoolein lipid comprises less than about 10 wt % of monolinolein. It may be that a monoolein lipid comprises less than about 5 wt % of monolinolein. It may be that a monoolein lipid comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 wt % monolinolein.

In some embodiments, it may be that a monoacylglycerol lipid comprises up to 100 wt % of monolinolein. It may be that a monoacylglycerol lipid comprises more than or equal to about 99 wt % of monolinolein. It may be that a monoacylglycerol lipid comprises more than about 90 wt % of monolinolein. It may be that a monoacylglycerol lipid comprises more than about 80 wt % of monolinolein. It may be that a monoacylglycerol lipid comprises more than about 70 wt % of monolinolein. It may be that a monoacylglycerol lipid comprises more than about 60 wt % of monolinolein. It may be that a monoacylglycerol lipid comprises more than about 50 wt % of monolinolein. It may be that a monoacylglycerol lipid comprises more than about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 wt % of monolinolein. It may be that a monoacylglycerol lipid comprises no monoolein. It may be that a monoacylglycerol lipid comprises less than about 50 wt % of monoolein. It may be that a monoacylglycerol lipid comprises less than about 40 wt % of monoolein. It may be that a monoacylglycerol lipid comprises less than about 30 wt % of monoolein. It may be that a monoacylglycerol lipid comprises less than about 20 wt % of monoolein. It may be that a monoacylglycerol lipid comprises less than about 10 wt % of monoolein. It may be that a monoacylglycerol lipid comprises less than about 5 wt % of monoolein. It may be that a monoacylglycerol lipid comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 wt % monoolein.

In other embodiments, it may be that a monoacylglycerol lipid comprises up to 100 wt % of monoolein. It may be that a monoacylglycerol lipid comprises more than or equal to about 99 wt % of monoolein. It may be that a monoacylglycerol lipid comprises more than about 90 wt % of monoolein. It may be that a monoacylglycerol lipid comprises more than about 80 wt % of monoolein. It may be that a monoacylglycerol lipid comprises more than about 70 wt % of monoolein. It may be that a monoacylglycerol lipid comprises more than about 60 wt % of monoolein. It may be that a monoacylglycerol lipid comprises more than about 50 wt % of monoolein. It may be that a monoacylglycerol lipid comprises more than about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 wt % of monoolein. It may be that a monoacylglycerol lipid comprises no monolinolein. It may be that a monoacylglycerol lipid comprises less than about 50 wt % of monolinolein. It may be that a monoacylglycerol lipid comprises less than about 40 wt % of monolinolein. It may be that a monoacylglycerol lipid comprises less than about 30 wt % of monolinolein. It may be that a monoacylglycerol lipid comprises less than about 20 wt % of monolinolein. It may be that a monoacylglycerol lipid comprises less than about 10 wt % of monolinolein. It may be that a monoacylglycerol lipid comprises less than about 5 wt % of monolinolein. It may be that a monoacylglycerol lipid comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 wt % monolinolein.

In embodiments, the lipid is a monoacylglycerol lipid. In preferred embodiments, the monoacylglycerol lipid is selected from monolinolein or monoolein. Thus, it may be that the lipid is monolinolein. It may be that the lipid is monoolein.

In embodiments, the lipid is a monoacylglycerol lipid. It may be that the monoacylglycerol lipid comprises monolinolein, or monoolein, or combinations thereof. Thus, it may be that the monoacylglycerol lipid comprises monolinolein and monoolein. It may be that the monoacylglycerol lipid comprises monolinolein. It may be that the monoacylglycerol lipid comprises monoolein. It may be that the monoacylglycerol lipid further comprises other lipids. It may be that the monoacylglycerol lipid further comprises up to about 10% of other lipids. It may be that the monoacylglycerol lipid further comprises up to about 8% of other lipids. It may be that the monoacylglycerol lipid further comprises up to about 5% of other lipids. It may be that the monoacylglycerol lipid further comprises about 1%, 2%, 3%, 4%, 5%, 6%, 7%, 8%, 9%, or 10% of other lipids. It may be that the monoacylglycerol lipid is substantially free of other lipids.

It may be that the monolinolein lipid comprises monoacylglycerol lipids. It may be that the monolinolein lipid comprises monoacylglycerol lipids and diglycerides. It may be that the monolinolein lipid comprises more than about 90 wt % of monoacylglycerol lipids. It may be that the monolinolein lipid comprises more than about 95 wt % of monoacylglycerol lipids. It may be that the monolinolein lipid comprises more than about 98 wt % of monoacylglycerol lipids. It may be that the monoacylglycerol lipids comprise C18 lipids. It may be that the monoacylglycerol lipids comprise more than about 80% of C18 lipids. It may be that the monoacylglycerol lipids comprise more than about 85% of C18 lipids. It may be that the monoacylglycerol lipids comprise more than about 90% of C18 lipids, for example 91% of C18 lipids. It may be that the C18 lipids comprise C18: 2, C18: 1 and/or C18: 0 lipids. It may be that the C18 lipids comprise more than about 50% of C18: 2 lipids. It may be that the C18 lipids comprise more than about 55% of C18: 2 lipids. It may be that the C18 lipids comprise more than about 60% of C18: 2 lipids. It may be that the C18 lipids comprise about 60% to about 65% of C18: 2 lipids, for example, about 60%, 60.5%, 61%, 61.5%, 61.6%, 61.7%, 61.8%, 61.9%, 62%, 62.1%, 62.2%, 62.3%, 62.4%, 62.5%, 63%, 63.5%, 64%, 64.5%, or 65% of C18.2 lipids. It may be that the C18 lipids comprise about 61.9% of C18: 2 lipids. It may be that the C18 lipids comprise more than about 15% of C18: 1 lipids. It may be that the C18 lipids comprise more than about 20% of C18: 1 lipids. It may be that the C18 lipids comprise about 25% of C18: 1 lipids. It may be that the C18 lipids comprise less than about 30% of C18: 1 lipids. It may be that the C18 lipids comprise about 20% to about 30% of C18: 1 lipids, for example, about 20%, 20.5%, 21%, 21.5%, 22%, 22.5%, 23%, 23.5%, 24%, 24.1%, 24.2%, 24.3%, 24.4% 24.5%, 24.6%, 24.7%, 24.8%, 24.9%, 25%, 25.5%, 26%, 26.5%, 27%, 27.5%, 28%, 28.5%, 29%, 29.5%, or 30% of C18.1 lipids. It may be that the C18 lipids comprise about 24.9% of C18: 1 lipids. It may be that the C18 lipids comprise more than about 1% of C18: 0 lipids. It may be that the C18 lipids comprise about 4% of C18: 0 lipids. It may be that the C18 lipids comprise about 5% of C18: 0 lipids. It may be that the C18 lipids comprise about less than 10% of C18: 0 lipids. It may be that the C18 lipids comprise about 1% to about 5% of C18: 0 lipids, for example, about 1%, 1.5%, 2%, 2.5%, 3%, 3.5%, 4%, 4.5%, or 5%, of C18.0 lipids. It may be that the C18 lipids comprise about 4.2% of C18: 1 lipids. It may be that the monoacylglycerol lipids comprise C16 lipids, for example, C16: 0 lipids. It may be that the monoacylglycerol lipids comprise more than about 1% of C16: 0 lipids. It may be that the monoacylglycerol lipids comprise more than about 5% of C16: 0 lipids. It may be that the monoacylglycerol lipids comprise more than about 7% of C16: 0 lipids. It may be that the monoacylglycerol lipids comprise less than about 10% of C16: 0 lipids. It may be that the monoacylglycerol lipids comprise about 5% to about 10% of C16: 0 lipids, for example, about 5%, 5.5%, 6%, 6.5%, 7%, 7.1%, 7.2%, 7.3%, 7.4%, 7.5%, 7.6%, 7.7%, 7.8%, 7.9%, 8%, 8.5%, 9%, 9.5%, or 10%, of C16.0 lipids. It may be that the monoacylglycerol lipids comprise about 7.4% of C16: 0 lipids. It may be that the monolinolein lipid comprises less than about 5% of diglycerides. It may be that the monolinolein lipid comprises less than about 3% of diglycerides. It may be that the monolinolein lipid comprises more than about 1% of diglycerides. For example, it may be that the monolinolein lipid comprises about 1%, 1.1%, 1.2%, 1.3%, 1.4%, 1.5%, 1.6%, 1.7%, 1.8%, 1.9%, 2%, 3%, 3.5%, 4%, 4.5%, or 5% of diglycerides. Thus, it may be that the monolinolein lipid comprises more than 98 wt % of monoacylglycerol lipids (comprising 61.9% of C18: 2 lipids, 24.9% of C18: 1 lipids, 4.2% of C18: 0 lipids, and 7.4% of C16: 0 lipids) and 1.6% of diglycerides.

Thus, in embodiments, provided herein is a composition comprising:
a) a carrier comprising:
   a1) water in an amount of more than 10% to 30% by weight of the carrier; and
   a2) lipid in an amount of 70% to 90% by weight of the carrier; and
b) a pharmaceutically active agent,
   wherein the lipid is selected from monolinolein or monoolein,
   wherein the composition forms a lipid cubic phase at a temperature of 36° C. to 39° C.

It may be that the composition comprises:
a) a carrier comprising:
   a1) water in an amount of more than 10% to 30% by weight of the carrier; and
   a2) monoacylglycerol lipid in an amount of 70% to 90% by weight of the carrier, wherein the monoacylglycerol lipid comprises monolinolein or monoolein, or combinations thereof; and
b) a pharmaceutically active agent,
   wherein the composition forms a lipid cubic phase at a temperature of 36° C. to 39° C.

It may be that the composition comprises:
a) a carrier comprising:
   a1) water in an amount of more than 10% to 25% by weight of the carrier; and
   a2) lipid in an amount of 75% to 90% by weight of the carrier; and
b) a pharmaceutically active agent,
   wherein the lipid is selected from monolinolein or monoolein,
   wherein the composition forms a lipid cubic phase at a temperature of 36° C. to 39° C. Preferably, the lipid is monolinolein.

It may be that the lipid is monolinolein. Thus, it may be that the composition comprises:
a) a carrier comprising:
   a1) water in an amount of more than 10% to 25% by weight of the carrier; and
   a2) monolinolein in an amount of 75% to 90% by weight of the carrier; and
b) a pharmaceutically active agent,
   wherein the composition forms a lipid cubic phase at a temperature of 36° C. to 39° C.

It may be that the lipid is monoolein. Thus, it may be that the composition comprises:
a) a carrier comprising:
   a1) water in an amount of more than 10% to 30% by weight of the carrier; and
   a2) monoolein in an amount of 70% to 90% by weight of the carrier; and
b) a pharmaceutically active agent,
   wherein the composition forms a lipid cubic phase at a temperature of 36° C. to 39° C. In preferred embodiments, the composition comprises up to 10% of the pharmaceutically active agent, wherein the % is % by weight based on the weight of the composition. It may be that the composition comprises from 0.1% to 10% of the pharmaceutically active agent, wherein the % is % by weight based on the weight of the composition. Thus, it may be that the composition comprises:
a) a carrier comprising:
   a1) water in an amount of more than 10% to 30% by weight of the carrier; and
   a2) lipid in an amount of 70% to 90% by weight of the carrier; and
b) a pharmaceutically active agent in an amount of 0.1% to 10% by weight of the composition,
   wherein the lipid is selected from monolinolein or monoolein,
   wherein the composition forms a lipid cubic phase at a temperature of 36° C. to 39° C.

It may be that the composition comprises:
a) a carrier comprising:
   a1) water in an amount of more than 10% to 30% by weight of the carrier; and
   a2) monoacylglycerol lipid in an amount of 70% to 90% by weight of the carrier, wherein the monoacylglycerol lipid comprises monolinolein or monoolein, or combinations thereof; and
b) a pharmaceutically active agent in an amount of 0.1% to 10% by weight of the composition,
   wherein the composition forms a lipid cubic phase at a temperature of 36° C. to 39° C.

Thus, it may be that the composition comprises:
a) a carrier comprising:
   a1) water in an amount of more than 10% to 25% by weight of the carrier; and
   a2) lipid in an amount of 75% to 90% by weight of the carrier; and
b) a pharmaceutically active agent in an amount of 0.1% to 10% by weight of the composition,
   wherein the lipid is selected from monolinolein or monoolein,
   wherein the composition forms a lipid cubic phase at a temperature of 36° C. to 39° C.

In embodiments, the composition comprises:
(i) 0.1% w/w to 10% w/w of a pharmaceutically active agent;
(ii) 67.5% w/w to 89.9% w/w lipid; and
(iii) 9% w/w to 24.9% w/w water,
wherein the lipid is selected from monolinolein or monoolein,
wherein the composition forms a lipid cubic phase at a temperature of 36° C. to 39° C.

In preferred embodiments, the composition comprises:
a) a carrier comprising:
   a1) water in an amount of from 14% to 18% by weight of the carrier; and
   a2) monoacylglycerol lipid in an amount of 82% to 86% by weight of the carrier, wherein the monoacylglycerol lipid comprises at least 50% by weight monolinolein; and
b) a pharmaceutically active agent in an amount of 0.1% to 10% by weight of the composition,
wherein the composition forms a lipid cubic phase at a temperature of 36° C. to 39° C.

In embodiments, the composition of the invention has a lamellar phase structure at 25° C. It may be that the composition of the invention has a lamellar phase structure at temperatures in the range of from greater than 25° C. to less than rectal temperature. For example, it may be that the composition of the invention has a lamellar phase structure at temperatures in the range of from greater than 25° C. to less than 38° C. Thus, it may be that the composition of the invention has a lamellar phase structure from 25° C. to 37° C. It may be that the composition of the invention has a lamellar phase structure from 25° C. to 36° C. It may be that the composition of the invention has a lamellar phase structure at 25° C., 26° C., 27° C., 28° C., 29° C., 30° C., 31° C., 32° C., 33° C., 34° C., 35° C., 36° C., or 37° C. 119. In embodiments, it may be that the composition is a lamellar gel at 25° C. It may be that the composition of the invention is a lamellar gel at temperatures in the range of from greater than 25° C. to less than rectal temperature. For example, it may be that the composition of the invention is a lamellar gel at temperatures in the range of from greater than 25° C. to less than 38° C. Thus, it may be that the composition of the invention is a lamellar gel at 25° C. to 37° C. It may be that the composition of the invention is a lamellar gel at from 25° C. to 36° C. It may be that the composition of the invention is a lamellar gel at 25° C., 26° C., 27° C., 28° C., 29° C., 30° C., 31° C., 32° C., 33° C., 34° C., 35° C., 36° C., or 37° C.

In embodiments, the lamellar gel has a low structural strength (as indicated by a lower value of storage modulus and loss modulus (G′ and G″, respectively)) with respect to the lipid cubic phase. Thus, the lamellar gel is less viscous than when the composition converts to the lipid cubic phase. Accordingly, the lamellar gel is easier to administer to the subject. Suitably the lamellar composition or gel has a viscosity at ambient temperature (e.g. 25° C.) suitable to be injected through a standard gauge needle (e.g. for subcutaneous administration), or via a conventional enema or rectal administration device.

The lamellar phase of the composition at ambient temperature (e.g. a lamellar gel) is more viscous than a simple aqueous solution of the pharmaceutically active agent. The higher viscosity of the lamellar composition of the invention at ambient temperature improves retention of the composition in a subject immediately following rectal administration whilst the composition undergoes transition to a higher viscosity phase as the temperature of the composition warms to the rectal temperature. The initial retention of the composition is therefore improved compared to the use of conventional simple aqueous solutions or suspensions of an active agent.

In embodiments, when the composition is in the lipid cubic phase, the composition has both a G′ and G″ greater than the G′ and G″ in the lamellar phase. It may be that the lamellar gel has both a G′ and G″ lower than the G′ and G″ in the lipid cubic phase. It may be that the composition is less viscous in the lamellar phase. It may be that the composition is more viscous in the lipid cubic phase. It may be that as the composition phase transitions from the lamellar phase to the lipid cubic phase, the viscosity of the composition increases. Accordingly, the lipid cubic phase structure forms a sustained release depot in situ.

In embodiments, the composition in the lipid cubic phase is retained in the rectum from about 10 min to about 24 h. It may be that the composition in the lipid cubic phase is retained in the rectum from about 20 min to about 12 h. It may be that the composition in the lipid cubic phase is retained in the rectum from about 30 min to about 6 h. It may be that the composition in the lipid cubic phase is retained in the rectum for about 10 min, 20 min, 30 min, 40 min, 50 min, 1 h, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, or 24 h. Preferably, it may be that the composition in the lipid cubic phase is retained in the rectum for at least 30 min.

In embodiments, the composition has a zero shear viscosity of from 1×10⁶ to 1×10⁷ mPa·s, measured at 25° C. and at a 0.01 shear rate (s⁻¹) with a stress-controlled rheometer (Modular Compact Rheometer MCR 72 from Anton Paar, Graz, Austria) used in cone-plate geometry, 0.993° angle, and 49.942 mm diameter. It may be that the composition has a zero shear viscosity of 1×10⁶ mPa·s, 2×10⁶ mPa·s, 3×10⁶ mPa·s, 4×10⁶ mPa·s, 5×10⁶ mPa·s, 6×10⁶ mPa·s, 7×10⁶ mPa·s, 8×10⁶ mPa·s, 9×10⁶ mPa·s, or 1×10⁷ mPa·s, measured at 25° C. and 0.01 s⁻¹. It may be that the composition has a zero shear viscosity of 1×10⁶ mPa·s, measured at 25° C. and 0.01 s⁻¹. It may be that the composition has a zero shear viscosity of 1×10⁷ mPa·s, measured at 25° C. and 0.01 s⁻¹.

In embodiments, the composition has a lamellar phase structure, and has a zero shear viscosity of from 1×10⁶ mPa·s to 1×10⁷ mPa·s, measured at 25° C. and at a 0.01 s⁻¹. It may be that the composition has a lamellar phase structure, and has a zero shear viscosity of 1×10⁶ mPa·s, 2×10⁶ mPa·s, 3×10⁶ mPa·s, 4×10⁶ mPa·s, 5×10⁶ mPa·s, 6×10⁶ mPa·s, 7×10⁶ mPa·s, 8×10⁶ mPa·s, 9×10⁶ mPa·s, or 1×10⁷ mPa·s, measured at 25° C. and 0.01 s⁻¹.

In embodiments, the composition has a lipid cubic phase structure, and has a viscosity of from about 1×10⁷ mPa's to about 1×10⁹ mPa·s, measured at 38° C. It may be that the composition has a lipid cubic phase structure, and has a viscosity of from about 1×10⁷ mPa·s to about 1×10⁸ mPa·s, measured at 38° C. It may be that the composition has a lipid cubic phase structure, and has a viscosity of from about 1×10⁸ mPa·s to about 1×10⁹ mPa·s, measured at 38° C.

In embodiments, the composition is an enema composition. Thus, in embodiments, the composition is rectally administered to the lower colon of a subject as an enema. Unit doses of enema formulations can be administered from pre-filled bags or syringes. It may be that the composition is rectally administered to the sigmoid colon, descending colon, and/or rectum of a subject as an enema.

The viscosity of the enema composition when measured at 25° C. is preferably 10,000 to 70,000 mPa's, more preferably 10,000 to 70,000 mPa·s, and most preferably 10,000 to 40,000 mPa·s. The pH is preferably 5.5 to 7.5, more preferably 6.5 to 7.5.

Once administered to a subject (e.g. as an enema), the composition with a lamellar phase structure, gradually absorbs heat (and the available amount of water) from the body, and converts into the lipid cubic phase, thus forming a controlled release depot in situ. It may be that the composition adheres to the walls of the colon, and thus forms a bioadhesive controlled release depot in situ. It may be that the composition adheres to the walls of the sigmoid colon, descending colon, and/or rectum, and thus forms a bioadhesive controlled release depot in situ.

In embodiments, the composition forms a lipid cubic phase in situ. The term ‘in situ’ may be considered to mean that the composition undergoes a phase transition from a lamellar phase structure to a lipid cubic phase once the composition has been administered to a subject, and the composition reaches a temperature of from 36° C. to 39° C., preferably 38° C. The phase transition of the composition from a lamellar phase structure to a lipid cubic phase can be measured with SAXS, and determined after equilibration at the required temperature.

Preferably, the composition is administered to a subject rectally (e.g. as an enema). Thus, ‘in situ’ may be considered to mean that the composition undergoes a phase transition from a lamellar phase structure to a lipid cubic phase once the composition has been injected into the rectum of a subject, and the composition reaches a temperature of from 36° C. to 39° C., preferably 38° C. (i.e. rectal temperature).

In embodiments, the composition forms a lipid cubic phase at a temperature of from about 36° C. to about 39° C. It may be that the composition forms a lipid cubic phase at a temperature of from 36° C. to 39° C., 37° C. to 39° C., or 37.5° C. to 38.5° C.

It may be that the composition forms a lipid cubic phase at rectal temperature. Thus, it may be that the composition forms a lipid cubic phase at a temperature of 36.0° C., 36.1° C., 36.2° C., 36.3° C., 36.4° C., 36.5° C., 36.6° C., 36.7° C., 36.8° C., 36.9° C., 37.0° C., 37.1° C., 37.2° C., 37.3° C., 37.4° C., 37.5° C., 37.6° C., 37.7° C., 37.8° C., 37.9° C., 38.0° C., 38.1° C., 38.2° C., 38.3° C., 38.4° C., 38.5° C., 38.6° C., 38.7° C., 38.8° C., 38.9° C., or 39.0° C. Preferably, it may be that the composition forms a lipid cubic phase at a temperature of 38° C.

In embodiments, the composition forms a lipid cubic phase after about 1 min, at a temperature of from about 36° C. to about 39° C. It may be that the composition forms a lipid cubic phase after about 1 min to 30 min, at a temperature of from about 36° C. to about 39° C. It may be that the composition forms a lipid cubic phase after about 1 min to 20 min, at a temperature of from about 36° C. to about 39° C. It may be that the composition forms a lipid cubic phase after about 1 min to 10 min, at a temperature of from about 36° C. to about 39° C. Suitably, it may be that the composition forms a lipid cubic phase after about 5 min at a temperature of about 38° C.

In embodiments, the composition transforms into a lipid cubic phase by contact with water, body fluid and/or other aqueous medium, at a temperature of from about 36° C. to about 39° C. It may be that the composition transforms into a lipid cubic phase by contact with water, body fluid and/or other aqueous medium, at a temperature of 38° C. It may be that the body fluid is a fluid from a mucosal surface, gastro-intestinal fluid, extra-vascular fluid, extracellular fluid, interstitial fluid or plasma,

In embodiments, the composition transforms in situ into a lipid cubic phase by contact with water, body fluid and/or other aqueous medium. Thus, it may be that the composition is administered to a subject (e.g. administered to the subject via the rectum, for example, as an enema), and the composition transforms from a lamellar phase structure into a lipid cubic phase structure, at a temperature of from about 36° C. to about 39° C., by contact with water, body fluid and/or other aqueous medium. It may be that the composition is administered to a subject via the rectum (e.g. as an enema), and the composition transforms from a lamellar phase structure into a lipid cubic phase structure, at rectal temperature, by contact with water, body fluid and/or other aqueous medium. It may be that the composition is administered to a subject via the rectum (e.g. as an enema), and the composition transforms from a lamellar phase structure into a lipid cubic phase structure, at a temperature of 38° C., by contact with water, body fluid and/or other aqueous medium.

In embodiments, the composition forms a lipid cubic phase at a pH range of from about 5 to about 9. It may be that the composition forms a lipid cubic phase at a pH range of from about 6 to about 8. It may be that the composition forms a lipid cubic phase at a pH range of from about 6.5 to about 7.5. Thus, it may be that the composition forms a lipid cubic phase at a pH of about 5, 5.5, 6, 6.5, 7, 7.5, 8, 8.5, or 9.

In embodiments, the composition is substantially free from organic solvents. Thus, it may be that the composition contains less than 10% of organic solvents. It may mean that the composition contains less than 5% of organic solvents. It may mean that the composition contains less than 1% of organic solvents. It may mean that the composition contains less than 0.01% of organic solvents. It may mean that the composition contains less than 0.001% of organic solvents. It may mean that the composition contains no detectable organic solvents.

In embodiments, the composition is substantially free from other lipids. Thus, it may be that the composition contains less than 10% of other lipids. It may mean that the composition contains less than 5% of other lipids. It may mean that the composition contains less than 1% of other lipids. It may mean that the composition contains less than 0.01% of other lipids. It may mean that the composition contains less than 0.001% of other lipids. It may mean that the composition contains no detectable other lipids.

It embodiments, the composition is substantially free from other lipid components. Thus, it may be that the composition contains less than 10% of other lipid components. It may mean that the composition contains less than 5% of other lipid components. It may mean that the composition contains less than 1% of other lipid components. It may mean that the composition contains less than 0.01% of other lipid components. It may mean that the composition contains less than 0.001% of other lipid components. It may mean that the composition contains no detectable other lipid components.

In embodiments, the composition is substantially free from additives. Thus, it may be that the composition contains less than 10% of additives. It may mean that the composition contains less than 5% of additives. It may mean that the composition contains less than 1% of additives. It may mean that the composition contains less than 0.01% of additives. It may mean that the composition contains less than 0.001% of additives. It may mean that the composition contains no detectable other additives.

### Carrier

In embodiments, the composition comprises a carrier, wherein the carrier comprises more than 10% to 30% water, and 70% to 90% lipid, wherein the % is % by weight based on the weight of the carrier. It may be that the carrier consists of more than 10% to 30% water, and 70% to 90% lipid, wherein the % is % by weight based on the weight of the carrier.

In embodiments, the composition comprises a carrier, wherein the carrier comprises more than 10% to 25% water, and 75% to 90% lipid, wherein the % is % by weight based on the weight of the carrier. In preferred embodiments, the carrier consists of more than 10% to 25% water, and 75% to 90% lipid, wherein the % is % by weight based on the weight of the carrier.

In embodiments, the lipid is a monoacylglycerol lipid. In embodiments, the monoacylglycerol lipid is selected from monolinolein or monoolein. Suitably, the lipid in the carrier is monolinolein. Thus, in embodiments, the carrier comprises more than 10% to 30% water, and 70% to 90% lipid, wherein the % is % by weight based on the weight of the carrier, and wherein the lipid is selected from monolinolein or monoolein. In preferred embodiments, the carrier consists of more than 10% to 30% water, and 70% to 90% lipid, wherein the % is % by weight based on the weight of the carrier, and wherein the lipid is selected from monolinolein or monoolein.

In embodiments, the lipid is a monoacylglycerol lipid. In embodiments, the monoacylglycerol lipid is selected from monolinolein or monoolein. In embodiments, the monoacylglycerol lipid comprises monolinolein, or monoolein, or combinations thereof. Suitably, the lipid in the carrier is monolinolein. Thus, in embodiments, the carrier comprises more than 10% to 30% water, and 70% to 90% lipid, wherein the % is % by weight based on the weight of the carrier, and wherein the lipid is selected from monolinolein or monoolein. in embodiments, the carrier comprises more than 10% to 30% water, and 70% to 90% lipid, wherein the % is % by weight based on the weight of the carrier, and wherein the lipid is a monoacylglycerol lipid comprising monolinolein, or monoolein, or combinations thereof. In preferred embodiments, the carrier consists of more than 10% to 30% water, and 70% to 90% lipid, wherein the % is % by weight based on the weight of the carrier, and wherein the lipid is selected from monolinolein or monoolein. It may be that the carrier consists of more than 10% to 30% water, and 70% to 90% lipid, wherein the % is % by weight based on the weight of the carrier, and wherein the lipid is a monoacylglycerol lipid comprising monolinolein, or monoolein, or combinations thereof.

In embodiments, the carrier comprises more than 10% to 30% water, wherein the % is % by weight based on the weight of the carrier. For example, it may be that the carrier comprises from 10% to 30% water, 20% to 30% water, 10% to 25% water, 11% to 20% water, or 14% to 18% water, wherein the % is % by weight based on the weight of the carrier. It may be that the carrier comprises 10%, 10.5%, 11%, 12%, 13%, 14%, 15%, 16%, 17%, 18%, 19%, 20%, 21%, 22%, 23%, 24%, 25%, 26%, 27%, 28%, 29%, or 30% water. Preferably, it may be that the carrier comprises 16% water.

In embodiments, the water used in the carrier is deionised water. In certain embodiments the water used for the carrier is ultrapure water (e.g. with a resistivity of greater than about 18 MΩ·cm at 25° C.). In certain embodiments the water used in the carrier is phosphate buffered saline. In embodiments, the water used in the carrier is water for injection (WFI).

In embodiments, the carrier comprises from 70% to 90% lipid, wherein the % is % by weight based on the weight of the carrier. It may be that the carrier comprises from 70% to 90% lipid, 75% to 90% lipid, 70% to 80% lipid, 80% to 90% lipid, or 84% to 88% lipid, wherein the % is % by weight based on the weight of the carrier. It may be that the carrier comprises 70%, 71%, 72%, 73%, 74%, 75%, 76%, 77%, 78%, 79%, 80%, 81%, 82%, 83%, 84%, 85%, 86%, 87%, 88%, 89%, or 90% lipid. Preferably, it may be that the carrier comprises 86% lipid.

In embodiments, the carrier comprises from 70% to 90% lipid, wherein the % is % by weight based on the weight of the carrier. It may be that the carrier comprises from 70% to 90% lipid, 75% to 90% lipid, 70% to 80% lipid, 80% to 90% lipid, or 84% to 88% lipid, wherein the % is % by weight based on the weight of the carrier. It may be that the carrier comprises from 70% to 90% lipid, 75% to 90% lipid, 70% to 80% lipid, 80% to 90% lipid, 82% to 86% lipid, or 84% to 88% lipid, wherein the % is % by weight based on the weight of the carrier. It may be that the carrier comprises 70%, 71%, 72%, 73%, 74%, 75%, 76%, 77%, 78%, 79%, 80%, 81%, 82%, 83%, 84%, 85%, 86%, 87%, 88%, 89%, or 90% lipid. It may be that the carrier comprises 86% lipid. Preferably, it may be that the carrier comprises 84% lipid.

In embodiments, it may be that the carrier comprises more than 10% to 20% water, and 80% to 90% lipid. In preferred embodiments, it may be that the carrier comprises 16% water, and 84% lipid.

In embodiments, it may be that the carrier comprises more than 10% to 20% water, and 80% to 90% lipid. It may be that the carrier comprises 14% to 18% water, and 82% to 86% lipid. In preferred embodiments, it may be that the carrier comprises 16% water, and 84% lipid.

In other embodiments, it may be that the carrier comprises from 20% to 30% water, and 70% to 80% lipid.

In embodiments, the carrier comprises from 75% to 90% monoacylglycerol lipid, wherein the % is % by weight based on the weight of the carrier, wherein the monoacylglycerol lipid comprises monolinolein or monoolein, or combinations thereof. It may be that the carrier comprises from 75% to 90% monoacylglycerol lipid, 80% to 90% monoacylglycerol lipid, or 84% to 88% monoacylglycerol lipid, wherein the % is % by weight based on the weight of the carrier. It may be that the carrier comprises from 75% to 90% monoacylglycerol lipid, 80% to 90% monoacylglycerol lipid, 82% to 86% monoacylglycerol lipid or 84% to 88% monoacylglycerol lipid, wherein the % is % by weight based on the weight of the carrier. It may be that the carrier comprises 75%, 76%, 77%, 78%, 79%, 80%, 81%, 82%, 83%, 84%, 85%, 86%, 87%, 88%, 89%, or 90% monoacylglycerol lipid, wherein the monoacylglycerol lipid comprises monolinolein or monoolein, or combinations thereof. It may be that the carrier comprises 86% monoacylglycerol lipid. Preferably, it may be that the carrier comprises 84% monoacylglycerol lipid.

In embodiments, the carrier comprises from 75% to 90% monolinolein, wherein the % is % by weight based on the weight of the carrier. It may be that the carrier comprises from 75% to 90% monolinolein, 80% to 90% monolinolein, or 84% to 88% monolinolein, wherein the % is % by weight based on the weight of the carrier. It may be that the carrier comprises 75%, 76%, 77%, 78%, 79%, 80%, 81%, 82%, 83%, 84%, 85%, 86%, 87%, 88%, 89%, or 90% monolinolein. Preferably, it may be that the carrier comprises 86% monolinolein.

In embodiments, the carrier comprises from 75% to 90% monolinolein, wherein the % is % by weight based on the weight of the carrier. It may be that the carrier comprises from 75% to 90% monolinolein, 80% to 90% monolinolein, or 84% to 88% monolinolein, wherein the % is % by weight based on the weight of the carrier. It may be that the carrier comprises from 75% to 90% monolinolein, 80% to 90% monolinolein, 82% to 86% monolinolein or 84% to 88% monolinolein, wherein the % is % by weight based on the weight of the carrier. It may be that the carrier comprises 75%, 76%, 77%, 78%, 79%, 80%, 81%, 82%, 83%, 84%, 85%, 86%, 87%, 88%, 89%, or 90% monolinolein. It may be that the carrier comprises 86% monolinolein. Preferably, it may be that the carrier comprises 84% monolinolein.

In embodiments, the composition comprises:
a) a carrier comprising:
   a1) water in an amount of more than 10% to 20% by weight of the carrier; and
   a2) monolinolein in an amount of 80% to 90% by weight of the carrier; and
b) a pharmaceutically active agent,
wherein the composition forms a lipid cubic phase at a temperature of 36° C. to 39° C.

In embodiments, the composition comprises:
a) a carrier comprising:
   a1) water in an amount of 16% by weight of the carrier; and
   a2) monolinolein in an amount of 84% by weight of the carrier; and
b) a pharmaceutically active agent,
wherein the composition forms a lipid cubic phase at a temperature of 36° C. to 39° C. Preferably, the composition forms a lipid cubic phase at the rectal temperature. Thus, it may be that the composition comprises:
a) a carrier comprising:
   a1) water in an amount of 16% by weight of the carrier; and
   a2) monolinolein in an amount of 84% by weight of the carrier; and
b) a pharmaceutically active agent,
wherein the composition forms a lipid cubic phase at a temperature of 38° C.

In embodiments, the carrier comprises from 70% to 90% monoolein, wherein the % is % by weight based on the weight of the carrier. It may be that the carrier comprises from 70% to 90% monoolein, 70% to 85% monoolein, or 70% to 80% monoolein, wherein the % is % by weight based on the weight of the carrier. It may be that the carrier comprises 70%, 71%, 72%, 73%, 74%, 75%, 76%, 77%, 78%, 79%, or 80% monoolein.

In embodiments, the composition comprises:
a) a carrier comprising:
   a1) water in an amount of more than 20% to 30% by weight of the carrier; and
   a2) monoolein in an amount of 70% to 80% by weight of the carrier; and
b) a pharmaceutically active agent,
wherein the composition forms a lipid cubic phase at a temperature of 36° C. to 39° C. In embodiments, the composition comprises:
a) a carrier comprising:
   a1) water in an amount of 20% by weight of the carrier; and
   a2) monoolein in an amount of 80% by weight of the carrier; and
b) a pharmaceutically active agent,
wherein the composition forms a lipid cubic phase at a temperature of 36° C. to 39° C. Preferably, it may be that the composition forms a lipid cubic phase at the rectal temperature. Thus, it may be that the composition comprises:
a) a carrier comprising:
   a1) water in an amount of 20% by weight of the carrier; and
   a2) monoolein in an amount of 80% by weight of the carrier; and
b) a pharmaceutically active agent,
wherein the composition forms a lipid cubic phase at a temperature of 38° C.

In embodiments, the composition comprises:
a) a carrier comprising:
   a1) water in an amount of more than 10% to 30% by weight of the carrier; and
   a2) monoacylglycerol lipid in an amount of 70% to 90% by weight of the carrier, wherein the monoacylglycerol lipid comprises monolinolein or monoolein, or combinations thereof; and
b) a pharmaceutically active agent,
wherein the composition forms a lipid cubic phase at a temperature of 36° C. to 39° C. It may be that the composition forms a lipid cubic phase at rectal temperature. It may be that the composition forms a lipid cubic phase at 38° C. It may be that the monoacylglycerol lipid is monolinolein.

In embodiments, the composition comprises:
a) a carrier comprising:
   a1) water in an amount of more than 10% to 20% by weight of the carrier; and
   a2) monoacylglycerol lipid in an amount of 80% to 90% by weight of the carrier, wherein the monoacylglycerol lipid comprises monolinolein or monoolein, or combinations thereof; and
b) a pharmaceutically active agent,
wherein the composition forms a lipid cubic phase at a temperature of 36° C. to 39° C. It may be that the composition forms a lipid cubic phase at rectal temperature. It may be that the composition forms a lipid cubic phase at 38° C. It may be that the monoacylglycerol lipid is monolinolein.

In embodiments, the composition comprises:
a) a carrier comprising:
   a1) water in an amount of more than 14% to 18% by weight of the carrier; and
   a2) monoacylglycerol lipid in an amount of 82% to 86% by weight of the carrier, wherein the monoacylglycerol lipid comprises monolinolein or monoolein, or combinations thereof; and
b) a pharmaceutically active agent,
wherein the composition forms a lipid cubic phase at a temperature of 36° C. to 39° C. It may be that the composition forms a lipid cubic phase at rectal temperature. It may be that the composition forms a lipid cubic phase at 38° C. It may be that the monoacylglycerol lipid is monolinolein.

In embodiments, the composition comprises:
a) a carrier comprising:
   a1) water in an amount of 16% by weight of the carrier; and
   a2) monoacylglycerol lipid in an amount of 84% by weight of the carrier, wherein the monoacylglycerol lipid comprises monolinolein or monoolein, or combinations thereof; and
b) a pharmaceutically active agent,
wherein the composition forms a lipid cubic phase at a temperature of 36° C. to 39° C. It may be that the composition forms a lipid cubic phase at rectal temperature. It may be that the composition forms a lipid cubic phase at 38° C. It may be that the monoacylglycerol lipid is monolinolein.

In embodiments, the composition comprises:
a) a carrier comprising:
   a1) water in an amount of from 14% to 18% by weight of the carrier; and
   a2) monoacylglycerol lipid in an amount of 82% to 86% by weight of the carrier, wherein the monoacylglycerol lipid comprises at least 50% by weight monolinolein; and
b) a pharmaceutically active agent in an amount of 0.1% to 10% by weight of the composition,
wherein the composition forms a lipid cubic phase at a temperature of 36° C. to 39° C. It may be that the composition forms a lipid cubic phase at rectal temperature. It may be that the composition forms a lipid cubic phase at 38° C. It may be that the monoacylglycerol lipid comprises at least 55% by weight monolinolein.

In embodiments, the composition comprises:
a) a carrier comprising:
   a1) water in an amount of from 14% to 18% by weight of the carrier; and
   a2) monoacylglycerol lipid in an amount of 82% to 86% by weight of the carrier, wherein the monoacylglycerol lipid comprises at least 50% by weight monolinolein; and
b) a pharmaceutically active agent in an amount of 1% to 5% by weight of the composition,
wherein the composition forms a lipid cubic phase at a temperature of 36° C. to 39° C. It may be that the composition forms a lipid cubic phase at rectal temperature. It may be that the composition forms a lipid cubic phase at 38° C. It may be that the monoacylglycerol lipid comprises at least 55% by weight monolinolein.

In embodiments, the composition comprises:
a) a carrier comprising:
   a1) water in an amount of 16% by weight of the carrier; and
   a2) monoacylglycerol lipid in an amount of 84% by weight of the carrier, wherein the monoacylglycerol lipid comprises at least 50% by weight monolinolein; and
b) a pharmaceutically active agent in an amount of 1% to 5% by weight of the composition,
wherein the composition forms a lipid cubic phase at a temperature of 36° C. to 39° C. It may be that the composition forms a lipid cubic phase at rectal temperature. It may be that the composition forms a lipid cubic phase at 38° C. It may be that the monoacylglycerol lipid comprises at least 55% by weight monolinolein, for example, at least about 60% by weight monolinolein.

### Pharmaceutically Active Agent

In embodiments, the composition comprises at least one pharmaceutically active agent. Thus, it may be that the composition comprises one pharmaceutically active agent. It may be that the composition comprises more than one pharmaceutically active agent. For example, the composition may comprise two pharmaceutically active agents. It may be that the composition comprises two or more pharmaceutically active agents. For example, it may be that the composition comprises three pharmaceutically active agents. It may be that the composition comprises four pharmaceutically active agents.

In embodiments, the composition comprises up to 20% w/w of the pharmaceutically active agent. Thus, it may be that the composition comprises from 0.1% to 20% of the pharmaceutically active agent, wherein the % is % by weight based on the weight of the composition. In preferred embodiments, the composition comprises up to 10% w/w of the pharmaceutically active agent. It may be that the composition comprises from 0.1% to 10% of the pharmaceutically active agent, wherein the % is % by weight based on the weight of the composition. Thus, it may be that the composition comprises:
a) a carrier comprising:
   a1) water in an amount of more than 10% to 30% by weight of the carrier; and
   a2) lipid in an amount of 70% to 90% by weight of the carrier; and
b) a pharmaceutically active agent in an amount of from 0.1% to 10% by weight of the composition,
wherein the lipid is selected from monolinolein or monoolein,
wherein the composition forms a lipid cubic phase at a temperature of 36° C. to 39° C.

It may be that the composition comprises from 0.1% to 10% of the pharmaceutically active agent, 0.5% to 10% of the pharmaceutically active agent, 0.5% to 7.5% of the pharmaceutically active agent, or 1% to 5% of the pharmaceutically active agent, wherein the % is % by weight based on the weight of the composition. For example, it may be that the composition comprises 0.1%, 0.25%, 0.5%, 0.75%, 1%, 2%, 3%, 4%, 5%, 6%, 7%, 8%, 9%, or 10% of the pharmaceutically active agent, wherein the % is % by weight based on the weight of the composition. Preferably, it may be that the composition comprises from 0.1% to 10% of the pharmaceutically active agent, wherein the % is % by weight based on the weight of the composition. More preferably, it may be that the composition comprises from 1% to 5% of the pharmaceutically active agent, wherein the % is % by weight based on the weight of the composition. Thus, it may be that the composition comprises:
a) a carrier comprising:
   a1) water in an amount of more than 10% to 30% by weight of the carrier; and
   a2) lipid in an amount of 70% to 90% by weight of the carrier; and
b) a pharmaceutically active agent in an amount of from 1% to 5% by weight of the composition,
wherein the lipid is selected from monolinolein or monoolein,
wherein the composition forms a lipid cubic phase at a temperature of 36° C. to 39° C. 170. It may be that the composition comprises:
a) a carrier comprising:
   a1) water in an amount of more than 10% to 25% by weight of the carrier; and
   a2) lipid in an amount of 75% to 90% by weight of the carrier; and
b) a pharmaceutically active agent in an amount of from 1% to 5% by weight of the composition,
wherein the lipid is selected from monolinolein or monoolein, preferably monolinolein,
wherein the composition forms a lipid cubic phase at a temperature of 36° C. to 39° C. In this instance the total composition comprises:
(i) 1% w/w to 5% w/w of a pharmaceutically active agent;
(ii) 71.25% w/w to 89.1% w/w lipid; and
(iii) 9.5% w/w to 24.75% w/w water.

For example, it may be that the composition comprises 1% of the pharmaceutically active agent, wherein the % is % by weight based on the weight of the composition. In preferred embodiments, it may be that the composition comprises: a) a carrier comprising: a1) water in an amount of 16% by weight of the carrier; and a2) lipid in an amount of 84% by weight of the carrier; and b) a pharmaceutically active agent in an amount of 1% of by weight of the composition. Thus, it may be that the composition comprises:
a) 99% of a carrier (wherein the % is % by weight of the composition), wherein the carrier comprises:
   a1) water in an amount of 16% by weight of the carrier; and
   a2) lipid in an amount of 84% by weight of the carrier; and
b) 1% of a pharmaceutically active agent (wherein the % is % by weight of the composition),
wherein the lipid is selected from monolinolein or monoolein,
wherein the composition forms a lipid cubic phase at a temperature of 36° C. to 39° C. Preferably, the lipid is monolinolein. In this instance the total composition comprises:
(i) 1% w/w of a pharmaceutically active agent;
(ii) 83.16% w/w monolinolein; and
(iii) 15.84% w/w water.

In another example, it may be that the composition comprises 5% of the pharmaceutically active agent, wherein the % is % by weight based on the weight of the composition. In preferred embodiments, it may be that the composition comprises: a) a carrier comprising: a1) water in an amount of 16% by weight of the carrier; and a2) lipid in an amount of 84% by weight of the carrier; and b) a pharmaceutically active agent in an amount of 5% by weight of the composition. Thus, it may be that the composition comprises:
a) 95% of a carrier (wherein the % is % by weight of the composition), wherein the carrier comprises:
   a1) water in an amount of 16% by weight of the carrier; and
   a2) lipid in an amount of 84% by weight of the carrier; and
b) 5% of a pharmaceutically active agent (wherein the % is % by weight of the composition),
wherein the lipid is selected from monolinolein or monoolein,
wherein the composition forms a lipid cubic phase at a temperature of 36° C. to 39° C. Preferably, the lipid is monolinolein. In this instance the total composition comprises:
(i) 5% w/w of a pharmaceutically active agent;
(ii) 79.8% w/w monolinolein; and
(iii) 15.2% w/w water.

In other embodiments, it may be that the composition comprises: a) a carrier comprising: a1) water in an amount of 20% by weight of the carrier; and a2) lipid in an amount of 80% by weight of the carrier; and b) a pharmaceutically active agent in an amount of 1% by weight of the composition. Thus, it may be that the composition comprises:
a) 99% of a carrier (wherein the % is % by weight of the composition), wherein the carrier comprises:
   a1) water in an amount of 20% by weight of the carrier; and
   a2) lipid in an amount of 80% by weight of the carrier; and
b) 1% of a pharmaceutically active agent (wherein the % is % by weight of the composition),
wherein the lipid is selected from monolinolein or monoolein,
wherein the composition forms a lipid cubic phase at a temperature of 36° C. to 39° C. Preferably, the lipid is monoolein. In this instance the total composition comprises:
(i) 1% w/w of a pharmaceutically active agent;
(ii) 79.2% w/w monoolein; and
(iii) 19.8% w/w water.

In another example, it may be that the composition comprises: a) a carrier comprising: a1) water in an amount of 20% by weight of the carrier; and a2) lipid in an amount of 80% by weight of the carrier; and b) a pharmaceutically active agent in an amount of 5% by weight of the composition. Thus, it may be that the composition comprises:
a) 95% of a carrier (wherein the % is % by weight of the composition), wherein the carrier comprises:
   a1) water in an amount of 20% by weight of the carrier; and
   a2) lipid in an amount of 80% by weight of the carrier; and
b) 5% of a pharmaceutically active agent (wherein the % is % by weight of the composition),
wherein the lipid is selected from monolinolein or monoolein,
wherein the composition forms a lipid cubic phase at a temperature of 36° C. to 39° C. Preferably, the lipid is monoolein. In this instance the total composition comprises:
(i) 5% w/w of a pharmaceutically active agent;
(ii) 76% w/w monolinolein; and
(iii) 19% w/w water.

In embodiments, the pharmaceutically active agent is selected from a hydrophilic pharmaceutically active agent, or a hydrophobic pharmaceutically active agent. Thus, it may be that the pharmaceutically active agent is a hydrophilic pharmaceutically active agent. Accordingly, it may be that the pharmaceutically active agent is water soluble. In other embodiments, it may be that the pharmaceutically active agent is a hydrophobic pharmaceutically active agent. Accordingly, it may be that the pharmaceutically active agent is a lipophilic pharmaceutically active agent.

In embodiments, the pharmaceutically active agent is dissolved in the carrier. In other embodiments, the pharmaceutically active agent is suspended in the carrier.

In embodiments, the pharmaceutically active agent is a hydrophilic pharmaceutically active agent, which is dissolved in water to provide a drug mixture, as described in detail in the ‘Method of Preparation’ section herein.

In embodiments, the pharmaceutically active agent is a hydrophobic pharmaceutically active agent, which is not dissolved in water. It may be that the hydrophobic pharmaceutically active agent is mixed with a lipid, and hydrated in water to provide a lipid-drug mixture, as described in detail in the ‘Method of Preparation’ section herein.

In some embodiments, the pharmaceutically active agent is water soluble. By “soluble”, it is meant that 1 g of the pharmaceutically active agent requires less than 10,000 mL, preferably less than 1,000 mL, more preferably less than 100 mL, even more preferably less than 30 mL or 10 ml of solvent to dissolve at a given pH (at 25.0±0.5° C.). It may be that by “soluble”, it is meant that the log P of the substance has a negative value. In embodiments, the pharmaceutically active agent is soluble in water at pH 7.0 and 25.0±0.5° C.

In other embodiments, the pharmaceutically active agent is water insoluble. By “insoluble” it is meant that 1 g of the pharmaceutically active agent requires more than 10,000 mL of solvent to dissolve at a given pH (e.g. at pH 7.0 and 25.0±0.5° C.). It may be that by “insoluble”, it is meant that the log P of the substance has a positive value.

The pharmaceutically active ingredient is not particularly limited and can be selected by the skilled person according to the needs.

In embodiments, the composition may be for the use in the treatment or prevention of IBD (particularly, ulcerative colitis). In embodiments, the composition may be for the use in inhibiting IBD (particularly, ulcerative colitis) or preventing progression of the disease. Accordingly, the composition may comprise at least one pharmaceutically active agent selected from the group consisting of anti-inflammatory agents (e.g. 5-ASA, 4-ASA, sulphasalazine and balsalazide); non-steroidal anti-inflammatory agents (e.g. ibuprofen and diclofenac); steroids (e.g. prednisolone; budesonide, hydrocortisone or fluticasone); immunosuppressants (e.g. azathioprine; cyclosporin; tacrolimus and methotrexate); antibiotics (e.g. metronidazole, ciprofloxacin, amoxicillin, tetracycline and sulphamethoxazole); and biological agents including peptides, proteins, antibodies and antibody fragments. Suitable examples of biological agents include alkaline phosphatase and anti-TNF antibodies such as infliximab, adalimumab, certolizumab pegol, golimumab and ustekinumab.

In embodiments, the pharmaceutically active agent is selected from the group consisting of biological agents, anti-inflammatory agents, corticosteroids, immunosuppressants, antifungal agents, antibiotics, antifibrotic agents, and anti-cancer agents. In embodiments, the pharmaceutically active agent is selected from the group consisting of biological agents (e.g. anti-TNF antibodies, IL-23 inhibitors, IL-12 inhibitors, TLR9 agonists, anti-MAdCAM antibodies, a human IL-22Fc fusion protein, interleukins, anti-β7 integrin antibodies, matrixmetalloproteinase 9 (MMP9) inhibitors), JAK inhibitors, PDE4 inhibitors, sphingosine-1-phosphate receptor modulators, anti-inflammatory agents, corticosteroids, immunosuppressants, antifungal agents, antibiotics, antifibrotic agents, and anti-cancer agents.

In embodiments, the pharmaceutically active agent is a biological agent, for example, a peptide, protein, antibody, or antibody fragment. In embodiments, the pharmaceutically active agent is an antibody or functional fragment thereof. In embodiments, the pharmaceutically active agent is a peptide.

In embodiments, the pharmaceutically active agent is an antibody or functional fragment thereof, and is suitable for use in the treatment of a gastrointestinal disease, for example an inflammatory bowel disease (IBD) (e.g. Crohn's disease or ulcerative colitis), cancer (e.g. colorectal cancer or small intestine cancer), celiac disease, or an infection (e.g. Clostridium difficile infection), more preferably an IBD.

The antibody or functional fragment thereof used in the composition is not particularly limited. In one embodiment, the antibody or functional fragment thereof is an antibody. In another embodiment, the antibody or functional fragment thereof is a functional fragment as defined herein. The antibody or functional fragment thereof may further comprise one or more modifications, e.g. in the form of added or substituted residues, that improve stability, specificity or targeting. These may include any such modifications that are known in the art.

The antigen against which the antibody or functional fragment is directed i.e. the immunogen, peptide, protein, or other molecular structure to which the antibody or functional fragment thereof can specifically bind, is not limited. In its most general form (and when no defined reference is mentioned), “specific to” or “specific binding” refers to the ability of the antibody or functional fragment thereof to discriminate between the target of interest and an unrelated biomolecule (e.g. for antibodies specific to human TNFα to discriminate between human TNFα and an unrelated biomolecule), as determined, for example, in accordance with specificity assay methods known in the art.

In embodiments, the antibody or functional fragment thereof is selected from antibodies specific to tumour necrosis factor alpha (TNFα) and functional fragments thereof, antibodies specific to a4137 integrin and functional fragments thereof, antibodies specific to CD3, CD4 or CD20 and functional fragments thereof, antibodies specific to interleukin 6 (IL-6), interleukin 12 (IL-12), interleukin 13 (IL-13), interleukin 23 (IL-23) or to their receptors and functional fragments thereof, antibodies specific to Janus kinase (JAK) and functional fragments thereof, antibodies specific to CXCL10/IP-10 and functional fragments thereof, and antibodies specific to p40 protein subunit and functional fragments thereof. In embodiments, the antibody or functional fragment thereof is selected from infliximab, adalimumab, etanercept, certolizumab pegol, golimumab, visilizumab, eldelumab, abrilumab, canakinumab, tocilizumab, ustekinumab, natalizumab, etrolizumab, priliximab, tofacitinib, or vedolizumab, and functional fragments thereof.

In embodiments, the antibody or functional fragment thereof in the composition specifically binds to TNFα. The terms “anti-TNFα antibody”, “TNFα antibody” and “antibody specific to TNFα” as used herein are interchangeable. In one embodiment, specific binding refers to the ability of the antibody or fragment to discriminate between human TNFα and human TNFβ. In a preferred embodiment of the present invention the TNFα antibody or functional fragment thereof is a TNFα antibody. In an alternatively preferred embodiment of the present invention the TNFα antibody or functional fragment thereof is a functional fragment of a TNFα antibody.

Currently approved anti-TNFα biotherapeutics include: (i) infliximab, a chimeric IgG anti-human monoclonal antibody (Remicade®); (ii) etanercept, a TNFR2 dimeric fusion protein, with an IgG1 Fc (Enbrel®); (iii) adalimumab, a fully human monoclonal antibody (mAb) (Humira®), (iv) certolizumab, a PEGylated Fab fragment (Cimzia®) and (v) golimumab, a human IgGIK monoclonal antibody (Simponi®). Therefore, in embodiments, the antibody or functional fragment thereof is selected from infliximab, adalimumab, etanercept, certolizumab pegol and golimumab or functional fragments thereof.

In embodiments, the pharmaceutically active agent is an anti-tumour necrosis factor-alpha inhibitor (TNF inhibitors). It may be that the TNF inhibitor is selected from adalimumab, certolizumab, and infliximab. In embodiments, the pharmaceutically active agent is an anti-integrin agent. It may be that the anti-integrin agent is selected from natalizumab and vedolizumab. In embodiments, the pharmaceutically active agent is an anti-interleukin-12 agent or an anti-interleukin-23 agent such as ustekinumab. In embodiments, the pharmaceutically active agent is a JAK inhibitor, such as tofacitinib. In preferred embodiments, the pharmaceutically active agent is tofacitinib.

In embodiments, the pharmaceutically active agent is an anti-inflammatory agent. It may be that the anti-inflammatory agent is a corticosteroid, such as prednisolone. Corticosteroids are steroids that help reduce both inflammation and immune response. It may be that the composition comprises a corticosteroid. It may be that the composition comprises a corticosteroid and a 5-ASA. Suitably, corticosteroids are not used as a long-term treatment to maintain remission of UC. Thus, it may be that a composition comprising a corticosteroid is for use as a short-term treatment for UC.

In embodiments, the pharmaceutically active agent is a corticosteroid selected from the group consisting of beclomethasone dipropionate, budesonide, hydrocortisone, methylprednisolone, prednisone, and prednisolone.

In embodiments, the pharmaceutically active agent is an anti-inflammatory agent, wherein the anti-inflammatory agent is an aminosalicylate. It is understood that aminosalicylates reduce inflammation in the intestine lining to ease symptoms of IBD (e.g. ulcerative colitis) and/or Crohn's. Suitably, it may be that the composition comprises an aminosalicylate, wherein the composition is for use in treating mild to moderate episodes of Crohn's.

In embodiments, the pharmaceutically active agent is an aminosalicylate selected from the group consisting of: balsalazide, mesalamine, olsalazine, and sulfasalazine.

It is understood that Crohn's disease is caused by a problem with the immune system, wherein cells that usually protect the body instead attack the GI tract. Accordingly, pharmaceutically active agents that suppress or regulate the immune system (i.e. immunosuppressants) are commonly used in the treatment of Crohn's.

In embodiments, the pharmaceutically active agent is an immunosuppressant. It may be that the composition is for use in the treatment of fistulas, e.g. IBD associated-perianal fistula, or vaginal fistula, wherein the composition comprises an immunosuppressant.

It may be that the subject is administered a composition comprising an immunosuppressant, as an alternative treatment to aminosalicylates and/or corticosteroids.

In embodiments, the pharmaceutically active agent is an immunosuppressant selected from the group consisting of: azathioprine, cyclosporine, mercaptopurine, methotrexate, mycophenolate mofetil, or tacrolimus. Preferably, it may be that the pharmaceutically active agent is tacrolimus.

In embodiments, it may be that the pharmaceutically active agent is an antifungal agent, for use in treating a fungal infection. It may be that the antifungal agent is:
(i) a polyene (e.g. amphotericin B (e.g. amphotericin B deoxycholate, liposomal amphotericin B, amphotericin B lipid complex or amphotericin B colloidal dispersion), candicidin, filipin, hamycin, natamycin, nystatin, or rimocidin);
(ii) a triazole, preferably a triazole other than a compound of the invention (e.g. albaconazole, efinaconazole, epoxiconazole, fluconazole, isavuconazole, itraconazole, posaconazole, propiconazole, ravuconazole, terconazole or voriconazole);
(iii) an imidazole (e.g. bifonazole, butoconazole, chlormidazole, clotrimazole, eberconazole, econazole, fenticonazole, flutrimazole, isoconazole, ketoconazole, luliconazole, miconazole, omoconazole, oxiconazole, sertaconazole, sulconazole, or tioconazole);
(iv) a thiazole (e.g. abafungin);
(v) an echinocandin (e.g. anidulafungin, biafungin, caspofungin, or micafungin);
(vi) an allylamine (e.g., amorolfin, butenafine, naftifine, or terbinafine); or
(vii) amorolfine, ciclopirox olamine, griseofulvin or flucytosine (5-fluorocytosine).

In certain embodiments the pharmaceutically active agent is an antifungal agent selected from clotrimazole, or fluconazole. Thus, it may be that the composition comprises clotrimazole. It may be that the composition comprises fluconazole.

In embodiments, it may be that the pharmaceutically active agent is an antibiotic. It may be that the composition comprising an antibiotic is for use in the treatment of a bacterial infection, e.g. a bacterial infection caused by Crohn's. For example, it may be that the bacterial infection caused by Crohn's is fistulas and/or abscesses.

In embodiments, the pharmaceutically active agent is an antibiotic selected from the group consisting of: ampicillin, ceftizoxime, sulbenicillin, piperacillin, mezlocillin, bacampicillin, cefoxitin, cefazolin, latamoxef, cefotaxime, ceftazidime, gentamicin, tobramycin, erythromycin, metronidazole, tinidazole, fluconazole, mupirocin, demeclocycline, retapamulin, chlortetracycline, virginiamycin, chloramphenicol, oxytetracycline, bacitracin, tetracycline, gentamicin, ciprofloxacin, rifaximin, and vancomycin.

In embodiments, it may be that the pharmaceutically active agent is an antifibrotic agent. Thus, it may be that the composition comprises an antifibrotic agent, wherein the antifibrotic agent is selected from pirfenidone or nintedanib.

In embodiments, the composition of the invention is for use in the treatment of a cancer. Suitably, the composition of the invention is for use in the treatment of a cancer affecting the GI tract, particularly the lower GI tract, and especially the colon. Accordingly, the composition may be for use in the treatment of colorectal cancer. Thus, the pharmaceutically active agent in the composition may be an anticancer agent. Alternatively, the composition may further comprise an anticancer agent.

Anti-cancer agents which may be suitable for use with the composition described herein include, but are not limited to one or more agents selected from:
(i) antiproliferative/antineoplastic drugs and combinations thereof, such as alkylating agents (for example cis-platin, oxaliplatin, carboplatin, cyclophosphamide, nitrogen mustard, uracil mustard, bendamustin, melphalan, chlorambucil, chlormethine, busulphan, temozolamide, nitrosoureas, ifosamide, melphalan, pipobroman, triethylene-melamine, triethylenethiophoporamine, carmustine, lomustine, stroptozocin and dacarbazine); antimetabolites (for example gemcitabine and antifolates such as fluoropyrimidines like 5-fluorouracil and tegafur, raltitrexed, methotrexate, pemetrexed, cytosine arabinoside, floxuridine, cytarabine, 6-mercaptopurine, 6-thioguanine, fludarabine phosphate, pentostatine, and gemcitabine and hydroxyurea); antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); proteasome inhibitors, for example carfilzomib and bortezomib; interferon therapy; and topoisomerase inhibitors (for example epipodophyllotoxins like etoposide and teniposide, amsacrine, topotecan, irinotecan, mitoxantrone and camptothecin); bleomcin, dactinomycin, daunorubicin, doxorubicin, epirubicin, idarubicin, ara-C, paclitaxel (Taxol™), nabpaclitaxel, docetaxel, mithramycin, deoxyco-formycin, mitomycin-C, L-asparaginase, interferons (especially IFN-alpha), etoposide, teniposide, VP-16, DNA-demethylating agents, (for example, azacitidine or decitabine); and histone de-acetylase (HDAC) inhibitors (for example vorinostat, MS-275, panobinostat, romidepsin, valproic acid, mocetinostat (MGCD0103) and pracinostat SB939);
(ii) cytostatic agents such as antiestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride; and navelbene, CPT-II, anastrazole, letrazole, capecitabine, reloxafme, cyclophosphamide, ifosamide, and droloxafine;
(iii) anti-invasion agents, for example dasatinib and bosutinib (SKI-606), and metalloproteinase inhibitors, inhibitors of urokinase plasminogen activator receptor function or antibodies to Heparanase;
(iv) inhibitors of growth factor function: for example such inhibitors include growth factor antibodies and growth factor receptor antibodies, for example the anti-erbB2 antibody trastuzumab [Herceptin™], the anti-EGFR antibody panitumumab, the anti-erbB1 antibody cetuximab, tyrosine kinase inhibitors, for example inhibitors of the epidermal growth factor family (for example EGFR family tyrosine kinase inhibitors such as gefitinib, erlotinib, 6-acrylamido-N-(3-chloro-4-fluorophenyl)-7-(3-morpholinopropoxy)-quinazolin-4-amine (CI 1033), erbB2 tyrosine kinase inhibitors such as lapatinib) and antibodies to costimulatory molecules such as CTLA-4, 4-IBB and PD-I, or antibodies to cytokines (IL-10, TGF-beta); inhibitors of the hepatocyte growth factor family; inhibitors of the insulin growth factor family; modulators of protein regulators of cell apoptosis (for example Bcl-2 inhibitors); inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib, tipifarnib and lonafarnib), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor, kinase inhibitors; aurora kinase inhibitors and cyclin dependent kinase inhibitors such as CDK2 and/or CDK4 inhibitors; and CCR2, CCR4 or CCR6 antagonists;
(v) antiangiogenic agents such as those which inhibit the effects of vascular endothelial growth factor, [for example the anti-vascular endothelial cell growth factor antibody bevacizumab (Avastin™)]; thalidomide; lenalidomide; and for example, a VEGF receptor tyrosine kinase inhibitor such as vandetanib, vatalanib, sunitinib, axitinib and pazopanib;
(vi) gene therapy approaches, including for example approaches to replace aberrant genes such as aberrant p53 or aberrant BRCA1 or BRCA2;
(vii) immunotherapy approaches, including for example antibody therapy such as alemtuzumab, rituximab, ibritumomab tiuxetan (Zevalin®) and ofatumumab; interferons such as interferon α; interleukins such as IL-2 (aldesleukin); interleukin inhibitors for example IRAK4 inhibitors; cancer vaccines including prophylactic and treatment vaccines such as HPV vaccines, for example Gardasil, Cervarix, Oncophage and Sipuleucel-T (Provenge); gp100; dendritic cell-based vaccines (such as Ad.p53 DC); toll-like receptor modulators for example TLR-7 or TLR-9 agonists; PD-1, PD-L1, PD-L2 and CTL4-A modulators (for example Nivolumab), antibodies and vaccines; other IDO inhibitors (such as indoximod); anti-PD-1 monoclonal antibodies (such as MK-3475 and nivolumab); anti-PDL1 monoclonal antibodies (such as MEDI-4736 and RG-7446); anti-PDL2 monoclonal antibodies; and anti-CTLA-4 antibodies (such as ipilumumab; and
(viii) cytotoxic agents for example fludaribine (fludara), cladribine, pentostatin (Nipent™).

In embodiments, the composition may be for the use in the prevention of colon cancer or colorectal cancer, primarily in patients suffering from colitis. Accordingly, the pharmaceutically active agent may be selected from the anti-inflammatory agents 5-ASA, sulindac, celecoxib and/or eflornithine (DFMO).

In embodiments the composition is administered to the subject as an enema. Thus, in embodiments, it may be that the pharmaceutically active agent is selected from the group consisting of mesalamine, budesonide, prednisolone, hydrocortisone, cobitolimod, tacrolimus, cyclosporin or tofacitinib.

Cytokine mediators of inflammation in IBD such as IL-9, IL-12, IL-23 and interferon-gamma (IFN-γ) are reliant on the Janus Kinase Signal Transducer and Activator of Transcription (JAK-STAT) pathway signalling. Therefore, targeting the JAK-STAT is an appealing therapeutic modality in IBD. JAK1, JAK2, JAK3 and tyrosine kinase 2 (TYK2) are all part of the JAK family of tyrosine kinase proteins. Thus, in embodiments, the pharmaceutically active agent is a JAK Inhibitor. In embodiments, the JAK inhibitor is selected from the group consisting of tofacitinib, filgotinib, upadacitinib, TD-1473, Brepocitinib (PF-06700841), or PF-06651600. In embodiments, the pharmaceutically active agent is a TYK2 inhibitor. In embodiments, the TYK2 inhibitor is Brepocitinib (PF-06700841), or BMS-986165.

IL-23 is a regulator of T-helper (Th)-17 cell and type 3 innate lymphoid cell (ILC3) pathways that lead to inflammatory cytokine production and inflammation and polymorphisms of the IL-23 receptor gene may be associated with increased susceptibility to Crohn's disease. IL-23 prevents regulatory T-cell response in the intestine, and therefore increases inflammation in the gut. In embodiments, the pharmaceutically active agent is an IL-23 inhibitor. In embodiments, the IL-23 inhibitor is selected from risankizumab, brazikumab, mirikizumab, or guselkumab.

In embodiments, the pharmaceutically active agent is an IL-6 inhibitor. It may be that the IL-6 inhibitor is PF-04236921.

In embodiments, the pharmaceutically active agent is a human IL-22Fc fusion protein. It may be that the human IL-22Fc fusion protein is UTTR1147A.

Migration of pro-inflammatory T cells into the gut facilitates inflammation which is characteristic of Crohn's disease and ulcerative colitis. Interaction between surface-expressed a4β1 and α4β7 integrins on lymphocytes and adhesion molecules, which are present on endothelial cells, allow for activated effector T cells to target the gut. Thus, in embodiments, it may be that the pharmaceutically active agent is an anti-adhesion molecule. It may be that the pharmaceutically active agent is an α4β7 integrin inhibitor. It may be that the pharmaceutically active agent is an α4β7 and αEβ7 integrin inhibitor. It may be that the pharmaceutically active agent is an α4 integrin inhibitor. It may be that the pharmaceutically active agent is selected from vedolizumab, etrolizumab, AJM300, Abrilumab (AMG 181 or MEDI 7183), or PF-00547659 (SHP647).

In embodiments, the pharmaceutically active agent is an anti-TNF agent. It may be that the anti-TNF agent is selected from AVX-470, or OPRX-106.

In embodiments, the pharmaceutically active agent is a sphingosine-1-phosphate receptor modulator (S1P1-S1P5). It may be that the S1P1-S1P5 is selected from ozanimod, etrasimod, or amiselimod (MT-1303).

In embodiments, the pharmaceutically active agent is a phosphodiesterase 4 (PDE4) inhibitor. It may be that the PDE4 inhibitor is apremilast.

In embodiments, the pharmaceutically active agent is a Toll-like-receptor 9 (TLR9) inhibitor. It may be that the TLR9 inhibitor is cobtiolimod.

In embodiments, the pharmaceutically active agent is selected from the agents listed in Table 1.

TABLE 1

| Drug class | Agent | Target |
|---|---|---|
| JAK inhibitors | Tofacitinib | JAK1/JAK3 |
| | Filgotinib | JAK1 |
| | Upadacitinib | JAK1 |
| | TD-1473 | Pan-JAK (gut selective) |
| | Brepocitinib | TYK2/JAK1 |
| | (PF-06700841) | |
| | PF-06651600 | JAK3 |
| | BMS-986165 | TYK2 |
| Anti-trafficking | Vedolizumab SC | α4β7 integrin |
| therapies | Etrolizumab | α4β7 and αEβ7 integrins |
| | AJM300 | α4 integrin |
| | PF-00547659 | MAdCAM |
| IL-23 inhibitors | Risankizumab | IL23/p19 subunit |
| | Brazikumab | IL23/p19 subunit |
| | Mirikizumab | IL23/p19 subunit |
| | Guselkumab | IL23/p19 subunit |
| S1P receptor | Ozanimod | S1PR1 and S1PR5 |
| modulators | Etrasimod | S1PR1, S1PR4, and S1PR5 |
| | Amiselimod (MT-1303) | S1PR1 |
| PDE4 inhibitors | Apremilast | PDE4 |
| TLR9 agonist | Cobitolimod | TLR9 |

Janus kinase (JAK), tyrosine kinase 2 (TYK 2), sphingosine 1 phosphate (S1P), sphingosine 1 phosphate receptor (S1PR), phosphodiesterase 4 (PDE4), toll-like receptor 9 (TLR9), alpha4-beta7 (α4β7), alpha4 (α4), alphaE-beta7 (αEβ7), mucosal addressin cell adhesion molecule-1 (MAdCAM), interleukin 23 (IL-23).

In embodiments, the pharmaceutically active agent is selected for use in the treatment of Crohn's disease and/or ulcerative colitis. Accordingly, it may be that the pharmaceutically active agent is selected from the group consisting of: ZEPOSIA® (ozanimod), HUMIRA® (Adalimumab), Hulio® (Adalimumab-fkjp), Avsola™ (Infliximab-axxq), Remicade® (infliximab), Abrilada™ (Adalimumab-afzb), HADLIMA (Adalimumab-bwwd), Hyrimoz (Adalimumab-adaz), Xeljanz® (Toficitinib), IXIFI™ (Infliximab-qbtx), CYLTEZO™ (Adalimumab-adbm), RENFLEXIS® (Infliximab-abda), STELARA® (Ustekinumab), AMJEVITA™ (Adalimumab-atto), MESALAMINE DR 800 mg, INFLECTRA™ (Infliximab-dyyb), UCERIS® (Budesonide), and ENTYVIO™ (Vedolizumab).

In embodiments, the pharmaceutically active agent is selected from the group consisting of: an antibody or functional fragment thereof, an anti-inflammatory agent, an anti-inflammatory agent, an immunosuppressant, an antifungal agent, an antibiotic, an antifibrotic agent, and an anticancer agent.

In embodiments, the pharmaceutically active agent is selected from the group consisting of: AbGn168H, ABT-494, ABX464, apremilast, PF-00547659, PF-06687234, 6-mercaptopurine, adalimumab, azathioprine, bertilimumab, brazikumab (MEDI2070), cobitolimod, certolizumab pegol, CP-690,550, corticosteroids (e.g., multimax budesonide, methylprednisolone), cyclosporine, E6007, etrolizumab, guselkumab, IL-2, etrasimod, figlotinib, golimumab, IMU-838, infliximab, matrixmetalloproteinase 9 (MMP9) inhibitors (e.g., GS-5745), mesalamine, mirikizumab (LY3074828), RPC 1063, risankizumab (BI 6555066), SHP647, sulfasalazine, TD-1473, TJ301, tildrakizumab (MK 3222), tacrolimus, Janus kinase inhibitors (e.g. tofacitinib), ustekinumab, UTTR1147A, vedolizumab, immunosuppressants (e.g. rapamycin), antifibrotics (e.g. pirfenidone, nintedanib) and antifungals (e.g. clotrimazole, fluconazole).

In preferred embodiments, the pharmaceutically active agent is a Janus kinase inhibitor. In embodiments, the pharmaceutically active agent is an inhibitor of the enzymes Janus kinase 1 (JAK 1) and/or 3 (JAK 3). Suitably, the Janus kinase inhibitor is tofacitinib (TOFA). It may be that the pharmaceutically active agent is tofacitinib, or a pharmaceutically acceptable salt thereof. Thus, it may be that the composition comprises tofacitinib, or a pharmaceutically acceptable salt thereof. In embodiments, the composition comprises up to 20% of tofacitinib, or a pharmaceutically acceptable salt thereof, wherein the % is % by weight based on the weight of the composition. In preferred embodiments, the composition comprises from 0.1% to 10% of tofacitinib, or a pharmaceutically acceptable salt thereof, wherein the % is % by weight based on the weight of the composition. It may be that the composition comprises 0.1% to 10% of tofacitinib, or a pharmaceutically acceptable salt thereof, 0.5% to 10% of tofacitinib, or a pharmaceutically acceptable salt thereof, 0.5% to 5% of tofacitinib, or a pharmaceutically acceptable salt thereof, or 1% to 5% of tofacitinib, or a pharmaceutically acceptable salt thereof, wherein the % is % by weight based on the weight of the composition. For example, it may be that the composition comprises 0.1%, 0.25%, 0.5%, 0.75%, 1%, 2%, 3%, 4%, 5%, 6%, 7%, 8%, 9% or 10% of tofacitinib, or a pharmaceutically acceptable salt thereof, wherein the % is % by weight based on the weight of the composition. Thus, it may be that the composition comprises:
a) a carrier comprising:
   a1) water in an amount of 10% to 30% by weight of the carrier; and
   a2) lipid in an amount of 70% to 90% by weight of the carrier; and
b) tofacitinib, or a pharmaceutically acceptable salt thereof in an amount from 0.1% to 10% by weight of the composition,
wherein the lipid is selected from monolinolein or monoolein,
wherein the composition forms a lipid cubic phase at a temperature of 36° C. to 39° C.

Suitably in this embodiment the lipid is monolinolein.

It may be that the composition comprises:
a) a carrier comprising:
   a1) water in an amount of 10% to 30% by weight of the carrier; and
   a2) monoacylglycerol lipid in an amount of 70% to 90% by weight of the carrier, wherein the monoacylglycerol lipid comprises monolinolein or monoolein, or combinations thereof; and
b) tofacitinib, or a pharmaceutically acceptable salt thereof in an amount from 0.1% to 10% by weight of the composition,
wherein the composition forms a lipid cubic phase at a temperature of 36° C. to 39° C.

It may be that the composition comprises:
a) a carrier comprising:
   a1) water in an amount of from 14% to 18% by weight of the carrier; and
   a2) monoacylglycerol lipid in an amount of 82% to 86% by weight of the carrier, wherein the monoacylglycerol lipid comprises at least 50% by weight monolinolein; and
b) tofacitinib, or a pharmaceutically acceptable salt thereof in an amount from 0.1% to 10% by weight of the composition,
wherein the composition forms a lipid cubic phase at a temperature of 36° C. to 39° C.

Preferably, it may be that the composition comprises from 1% to 5% of tofacitinib, or a pharmaceutically acceptable salt thereof, wherein the % is % by weight based on the weight of the composition. Thus, it may be that the composition comprises:
a) a carrier comprising:
   a1) water in an amount of more than 10% to 25% by weight of the carrier; and
   a2) lipid in an amount of 75% to 90% by weight of the carrier; and
b) tofacitinib, or a pharmaceutically acceptable salt thereof in an amount of from 1% to 5% by weight of the composition,
wherein the lipid is selected from monolinolein or monoolein,
wherein the composition forms a lipid cubic phase at a temperature of 36° C. to 39° C.

More preferably, it may be that the composition comprises 5% of tofacitinib, or a pharmaceutically acceptable salt thereof, wherein the % is % by weight based on the weight of the composition. Thus, in preferred embodiments, it may be that the composition comprises:
a) a carrier comprising:
   a1) water in an amount of 16% by weight of the carrier; and
   a2) monolinolein in an amount of 84% by weight of the carrier; and
b) tofacitinib, or a pharmaceutically acceptable salt thereof in an amount of 5% by weight of the composition,
wherein the composition forms a lipid cubic phase at a temperature of 36° C. to 39° C. It may be that the composition forms a lipid cubic phase at a temperature of 38° C. For the avoidance of doubt, in this embodiment, the total composition comprises:
(i) 5% w/w of tofacitinib, or a pharmaceutically acceptable salt thereof;
(ii) 79.8% w/w monolinolein; and
(iii) 15.2% w/w water,
i.e., the composition comprises 5 mg of TOFA per 100 mg of carrier.

In other preferred embodiments, the pharmaceutically active agent is tacrolimus (TAC). Thus, it may be that the composition comprises tacrolimus. In embodiments, the composition comprises up to 20% of tacrolimus, wherein the % is % by weight based on the weight of the composition. In preferred embodiments, the composition comprises from 0.1% to 10% of tacrolimus, wherein the % is % by weight based on the weight of the composition. It may be that the composition comprises 0.1% to 10% of tacrolimus, 0.5% to 10% of tacrolimus, 0.5% to 5% of tacrolimus, or 1% to 5% of tacrolimus, wherein the % is % by weight based on the weight of the composition. For example, it may be that the composition comprises 0.1%, 0.25%, 0.5%, 0.75%, 1%, 2%, 3%, 4%, 5%, 6%, 7%, 8%, 9% or 10% of tacrolimus, wherein the % is % by weight based on the weight of the composition. Thus, it may be that the composition comprises:
a) a carrier comprising:
   a1) water in an amount of more than 10% to 30% by weight of the carrier; and
   a2) lipid in an amount of 70% to 90% by weight of the carrier; and
b) tacrolimus in an amount of from 0.1% to 10% by weight of the composition,
wherein the lipid is selected from monolinolein or monoolein,
wherein the composition forms a lipid cubic phase at a temperature of 36° C. to 39° C.

Suitably in this embodiment the lipid is monolinolein.

It may be that the composition comprises:
a) a carrier comprising:
   a1) water in an amount of 10% to 30% by weight of the carrier; and
   a2) monoacylglycerol lipid in an amount of 70% to 90% by weight of the carrier, wherein the monoacylglycerol lipid comprises monolinolein or monoolein, or combinations thereof; and
b) tacrolimus in an amount of from 0.1% to 10% by weight of the composition,
wherein the composition forms a lipid cubic phase at a temperature of 36° C. to 39° C.

It may be that the composition comprises:
a) a carrier comprising:
   a1) water in an amount of from 14% to 18% by weight of the carrier; and
   a2) monoacylglycerol lipid in an amount of 82% to 86% by weight of the carrier, wherein the monoacylglycerol lipid comprises at least 50% by weight monolinolein; and
b) tacrolimus in an amount of from 0.1% to 10% by weight of the composition,
wherein the composition forms a lipid cubic phase at a temperature of 36° C. to 39° C.

Preferably, it may be that the composition comprises from 1% to 5% of tacrolimus, wherein the % is % by weight based on the weight of the composition. Thus, it may be that the composition comprises:
a) a carrier comprising:
   a1) water in an amount of more than 10% to 25% by weight of the carrier; and
   a2) lipid in an amount of 75% to 90% by weight of the carrier; and
b) tacrolimus in an amount of from 1% to 5% by weight of the composition,
wherein the lipid is selected from monolinolein or monoolein,
wherein the composition forms a lipid cubic phase at a temperature of 36° C. to 39° C.

More preferably, it may be that the composition comprises 1% of tacrolimus, wherein the % is % by weight based on the weight of the composition. Thus, in preferred embodiments, it may be that the composition comprises:
a) a carrier comprising:
   a1) water in an amount of 16% by weight of the carrier; and
   a2) monolinolein in an amount of 84% by weight of the carrier; and
b) tacrolimus in an amount of 1% by weight of the composition,
wherein the composition forms a lipid cubic phase at a temperature of 36° C. to 39° C. It may be that the composition forms a lipid cubic phase at a temperature of 38° C. For the avoidance of doubt, in this embodiment, the total composition comprises:
(i) 1% w/w of tacrolimus;
(ii) 83.16% w/w monolinolein; and
(iii) 15.84% w/w water.
i.e., the composition comprises 1 mg of TAC per 100 mg of carrier.

In other embodiments, the pharmaceutically active agent is clotrimazole. Thus, it may be that the composition comprises clotrimazole. In embodiments, the composition comprises up to 20% of clotrimazole, wherein the % is % by weight based on the weight of the composition. In preferred embodiments, the composition comprises from 0.1% to 10% of clotrimazole, wherein the % is % by weight based on the weight of the composition. It may be that the composition comprises 0.1% to 10% of clotrimazole, 0.5% to 10% of clotrimazole, 0.5% to 5% of clotrimazole, or 1% to 5% of clotrimazole, wherein the % is % by weight based on the weight of the composition. For example, it may be that the composition comprises 0.1%, 0.25%, 0.5%, 0.75%, 1%, 2%, 3%, 4%, 5%, 6%, 7%, 8%, 9% or 10% of clotrimazole, wherein the % is % by weight based on the weight of the composition. Thus, it may be that the composition comprises:
a) a carrier comprising:
   a1) water in an amount of more than 10% to 30% by weight of the carrier; and
   a2) lipid in an amount of 70% to 90% by weight of the carrier; and
b) clotrimazole in an amount of from 0.1% to 10% by weight of the composition,
wherein the lipid is selected from monolinolein or monoolein,
wherein the composition forms a lipid cubic phase at a temperature of 36° C. to 39° C. Suitably in this embodiment the lipid is monolinolein.

It may be that the composition comprises:
a) a carrier comprising:
   a1) water in an amount of 10% to 30% by weight of the carrier; and
   a2) monoacylglycerol lipid in an amount of 70% to 90% by weight of the carrier, wherein the monoacylglycerol lipid comprises monolinolein or monoolein, or combinations thereof; and
b) clotrimazole in an amount of from 0.1% to 10% by weight of the composition,
wherein the composition forms a lipid cubic phase at a temperature of 36° C. to 39° C.

It may be that the composition comprises:
a) a carrier comprising:
   a1) water in an amount of from 14% to 18% by weight of the carrier; and
   a2) monoacylglycerol lipid in an amount of 82% to 86% by weight of the carrier, wherein the monoacylglycerol lipid comprises at least 50% by weight monolinolein; and
b) clotrimazole in an amount of from 0.1% to 10% by weight of the composition,
wherein the composition forms a lipid cubic phase at a temperature of 36° C. to 39° C.

Preferably, it may be that the composition comprises from 1% to 5% of clotrimazole, wherein the % is % by weight based on the weight of the composition. Thus, it may be that the composition comprises:
a) a carrier comprising:
   a1) water in an amount of more than 10% to 25% by weight of the carrier; and
   a2) lipid in an amount of 75% to 90% by weight of the carrier; and
b) clotrimazole in an amount of from 1% to 5% by weight of the composition,
wherein the lipid is selected from monolinolein or monoolein,
wherein the composition forms a lipid cubic phase at a temperature of 36° C. to 39° C.

More preferably, it may be that the composition comprises 5% of clotrimazole, wherein the % is % by weight based on the weight of the composition. Thus, in preferred embodiments, it may be that the composition comprises:
a) a carrier comprising:
   a1) water in an amount of 16% by weight of the carrier; and
   a2) monolinolein in an amount of 84% by weight of the carrier; and
b) clotrimazole in an amount of 5% by weight of the composition,
wherein the composition forms a lipid cubic phase at a temperature of 36° C. to 39° C. It may be that the composition forms a lipid cubic phase at a temperature of 38° C. For the avoidance of doubt, in this embodiment, the total composition comprises:
(i) 5% w/w of clotrimazole;
(ii) 79.8% w/w monolinolein; and
(iii) 15.2% w/w water.
i.e., the composition comprises 5 mg of clotrimazole per 100 mg of carrier.

In embodiments, the pharmaceutically active agent is mesalamine. It will be understood that mesalamine is also known as 5-aminosalicylic acid (5-ASA). Thus, it may be that the pharmaceutically active agent is 5-ASA.

In other embodiments, the pharmaceutically active agent is mesalamine. Thus, it may be that the composition comprises mesalamine. In embodiments, the composition comprises up to 20% of mesalamine, wherein the % is % by weight based on the weight of the composition. In preferred embodiments, the composition comprises from 0.1% to 10% of mesalamine, wherein the % is % by weight based on the weight of the composition. It may be that the composition comprises 0.1% to 10% of mesalamine, 0.5% to 10% of mesalamine, 0.5% to 5% of mesalamine, or 1% to 5% of mesalamine, wherein the % is % by weight based on the weight of the composition. For example, it may be that the composition comprises 0.1%, 0.25%, 0.5%, 0.75%, 1%, 2%, 3%, 4%, 5%, 6%, 7%, 8%, 9% or 10% of mesalamine, wherein the % is % by weight based on the weight of the composition. Thus, it may be that the composition comprises:
a) a carrier comprising:
   a1) water in an amount of more than 10% to 30% by weight of the carrier; and
   a2) lipid in an amount of 70% to 90% by weight of the carrier; and
b) mesalamine in an amount of from 0.1% to 10% by weight of the composition,
wherein the lipid is selected from monolinolein or monoolein,
wherein the composition forms a lipid cubic phase at a temperature of 36° C. to 39° C.

Suitably in this embodiment the lipid is monolinolein.

It may be that the composition comprises:
a) a carrier comprising:
   a1) water in an amount of 10% to 30% by weight of the carrier; and
   a2) monoacylglycerol lipid in an amount of 70% to 90% by weight of the carrier, wherein the monoacylglycerol lipid comprises monolinolein or monoolein, or combinations thereof; and
b) mesalamine in an amount of from 0.1% to 10% by weight of the composition,
wherein the composition forms a lipid cubic phase at a temperature of 36° C. to 39° C.

It may be that the composition comprises:
a) a carrier comprising:
   a1) water in an amount of from 14% to 18% by weight of the carrier; and
   a2) monoacylglycerol lipid in an amount of 82% to 86% by weight of the carrier, wherein the monoacylglycerol lipid comprises at least 50% by weight monolinolein; and
b) mesalamine in an amount of from 0.1% to 10% by weight of the composition,
wherein the composition forms a lipid cubic phase at a temperature of 36° C. to 39° C.

Preferably, it may be that the composition comprises from 1% to 5% of mesalamine, wherein the % is % by weight based on the weight of the composition. Thus, it may be that the composition comprises:
a) a carrier comprising:
   a1) water in an amount of more than 10% to 25% by weight of the carrier; and
   a2) lipid in an amount of 75% to 90% by weight of the carrier; and
b) mesalamine in an amount of from 1% to 5% by weight of the composition,
wherein the lipid is selected from monolinolein or monoolein,
wherein the composition forms a lipid cubic phase at a temperature of 36° C. to 39° C.

More preferably, it may be that the composition comprises 5% of mesalamine, wherein the % is % by weight based on the weight of the composition. Thus, in preferred embodiments, it may be that the composition comprises:
a) a carrier comprising:
   a1) water in an amount of 16% by weight of the carrier; and
   a2) monolinolein in an amount of 84% by weight of the carrier; and
b) mesalamine in an amount of 5% by weight of the composition,
wherein the composition forms a lipid cubic phase at a temperature of 36° C. to 39° C. It may be that the composition forms a lipid cubic phase at a temperature of 38° C. For the avoidance of doubt, in this embodiment, the total composition comprises:
(i) 5% w/w of mesalamine;
(ii) 79.8% w/w monolinolein; and
(iii) 15.2% w/w water.
i.e., the composition comprises 5 mg of mesalamine per 100 mg of carrier.

In other embodiments, the pharmaceutically active agent is budesonide. Thus, it may be that the composition comprises budesonide. In embodiments, the composition comprises up to 20% of budesonide, wherein the % is % by weight based on the weight of the composition. In preferred embodiments, the composition comprises from 0.1% to 10% of budesonide, wherein the % is % by weight based on the weight of the composition. It may be that the composition comprises 0.1% to 10% of budesonide, 0.5% to 10% of budesonide, 0.5% to 5% of budesonide, or 1% to 5% of budesonide, wherein the % is % by weight based on the weight of the composition. For example, it may be that the composition comprises 0.1%, 0.25%, 0.5%, 0.75%, 1%, 2%, 3%, 4%, 5%, 6%, 7%, 8%, 9% or 10% of budesonide, wherein the % is % by weight based on the weight of the composition. Thus, it may be that the composition comprises:
a) a carrier comprising:
   a1) water in an amount of more than 10% to 30% by weight of the carrier; and
   a2) lipid in an amount of 70% to 90% by weight of the carrier; and
b) budesonide in an amount of from 0.1% to 10% by weight of the composition,
wherein the lipid is selected from monolinolein or monoolein,
wherein the composition forms a lipid cubic phase at a temperature of 36° C. to 39° C.

Suitably in this embodiment the lipid is monolinolein.

It may be that the composition comprises:
a) a carrier comprising:
   a1) water in an amount of 10% to 30% by weight of the carrier; and
   a2) monoacylglycerol lipid in an amount of 70% to 90% by weight of the carrier, wherein the monoacylglycerol lipid comprises monolinolein or monoolein, or combinations thereof; and
b) budesonide in an amount of from 0.1% to 10% by weight of the composition,
wherein the composition forms a lipid cubic phase at a temperature of 36° C. to 39° C.

It may be that the composition comprises:
a) a carrier comprising:
   a1) water in an amount of from 14% to 18% by weight of the carrier; and
   a2) monoacylglycerol lipid in an amount of 82% to 86% by weight of the carrier, wherein the monoacylglycerol lipid comprises at least 50% by weight monolinolein; and
b) budesonide in an amount of from 0.1% to 10% by weight of the composition,
wherein the composition forms a lipid cubic phase at a temperature of 36° C. to 39° C.

Preferably, it may be that the composition comprises from 1% to 5% of budesonide, wherein the % is % by weight based on the weight of the composition. Thus, it may be that the composition comprises:
a) a carrier comprising:
   a1) water in an amount of more than 10% to 25% by weight of the carrier; and
   a2) lipid in an amount of 75% to 90% by weight of the carrier; and
b) budesonide in an amount of from 1% to 5% by weight of the composition,
wherein the lipid is selected from monolinolein or monoolein,
wherein the composition forms a lipid cubic phase at a temperature of 36° C. to 39° C.

More preferably, it may be that the composition comprises 5% of budesonide, wherein the % is % by weight based on the weight of the composition. Thus, in preferred embodiments, it may be that the composition comprises:
a) a carrier comprising:
   a1) water in an amount of 16% by weight of the carrier; and
   a2) monolinolein in an amount of 84% by weight of the carrier; and
b) budesonide in an amount of 5% by weight of the composition,
wherein the composition forms a lipid cubic phase at a temperature of 36° C. to 39° C. It may be that the composition forms a lipid cubic phase at a temperature of 38° C. For the avoidance of doubt, in this embodiment, the total composition comprises:
(i) 5% w/w of budesonide;
(ii) 79.8% w/w monolinolein; and
(iii) 15.2% w/w water.
i.e., the composition comprises 5 mg of budesonide per 100 mg of carrier.

### Other Additives

Additives can modify the structure of the lipid mesophases. For example, adding an increasing amount of hexadecane or vitamin A can tune the phase of a monoolein-water system. The geometry of self-assembled mesophases is important in determining the release rate, and as such, the open or closed state of the aqueous channels influences the rate of drug release. Typical lipidic mesophases with a Pn3m, Im3m, or Ia3d symmetry are characterised by water channels with a diameter of about 3 to 5 nm. This geometric constraint prevents large hydrophilic molecules, such as hydrophilic proteins, hormones, and anti-bodies, from being included in the mesophase. However, this structural limitation can be overcome by additives that increase the water-channel dimensions, including hydration-modulating agents such as sucrose stearate, phospholipids, and cholesterol. Electrostatic swelling increases the size of the water channels, for example, by doping lipids with charged lipids capable of swelling the mesophases.

Thus, in embodiments, the composition further comprises an additive. It may be that the composition comprises at least one additive. Thus, it may be that the composition comprises one additive. It may be that the composition comprises more than one additive. For example, the composition may comprise two additives. It may be that the composition comprises three additives. It may be that the composition comprises four additives.

In embodiments, the composition further comprises up to 10% of an additive, wherein the % is % by weight based on the weight of the composition. Thus, it may be that the composition comprises from 0.1% to 10% of the additive, wherein the % is % by weight based on the weight of the composition. It may be that the composition comprises 0.1%, 0.25%, 0.5%, 0.75%, 1%, 2%, 3%, 4%, 5%, 6%, 7%, 8%, 9% or 10% of the additive, wherein the % is % by weight based on the weight of the composition.

In embodiments, the additive is selected from the group consisting of vitamin A, sucrose stearate, phospholipids, cholesterol, and electrolytes (e.g. sodium chloride, sodium sulfate, sodium iodide and calcium cations).

In embodiments, the additive is a negatively or positively charged phospholipid. Thus, it may be that the additive is selected from the group consisting of: 1, 2-dioleoyl-sn-glycero-3-phospho-(1′-rac-glycerol) (DOPG), 1,2-dioleoyl-sn-glycero-3-phospho-L-serine (DOPS), and 2-dioleoyl-3 trimethylammonium propane (DOTAP). It may be that the composition further comprises cholesterol, and a negatively or positively charged phospholipid (e.g. DOPG, DOPS, and DOTAP).

In embodiments, the additive is a negatively or positively charged phospholipid (e.g. DOPG, DOPS, and DOTAP). Thus, it may be that the composition further comprises up to 10% of a negatively or positively charged phospholipid (e.g. DOPG, DOPS, or DOTAP), wherein the % is % by weight based on the weight of the composition. Thus, it may be that the composition further comprises from 0.1% to 10% of a negatively or positively charged phospholipid (e.g. DOPG, DOPS, or DOTAP), wherein the % is % by weight based on the weight of the composition. It may be that the composition comprises 0.1%, 0.25%, 0.5%, 0.75%, 1%, 2%, 3%, 4%, 5%, 6%, 7%, 8%, 9% or 10% of a negatively or positively charged phospholipid (e.g. DOPG, DOPS, or DOTAP), wherein the % is % by weight based on the weight of the composition.

In embodiments, the additive is cholesterol. Thus, it may be that the composition further comprises up to 5% of cholesterol, wherein the % is % by weight based on the weight of the composition. Thus, it may be that the composition further comprises from 0.1% to 5% of cholesterol, wherein the % is % by weight based on the weight of the composition. It may be that the composition comprises 0.1%, 0.25%, 0.5%, 0.75%, 1%, 2%, 3%, 4%, or 5% of cholesterol, wherein the % is % by weight based on the weight of the composition.

In embodiments, the composition further comprises cholesterol, and a negatively or positively charged phospholipid (e.g. DOPG, DOPS, and DOTAP). It may be that the composition further comprises up to 5% of cholesterol, and up to 10% of a negatively or positively charged phospholipid (e.g. DOPG, DOPS, or DOTAP), wherein the % is % by weight based on the weight of the composition. Thus, it may be that the composition further comprises from 0.1% to 5% of cholesterol, and from 0.1% to 10% of a negatively or positively charged phospholipid (e.g. DOPG, DOPS, or DOTAP), wherein the % is % by weight based on the weight of the composition. It may be that the composition comprises 0.1%, 0.25%, 0.5%, 0.75%, 1%, 2%, 3%, 4%, or 5% of cholesterol, and 0.1%, 0.25%, 0.5%, 0.75%, 1%, 2%, 3%, 4%, 5%, 6%, 7%, 8%, 9% or 10% of a negatively or positively charged phospholipid (e.g. DOPG, DOPS, or DOTAP), wherein the % is % by weight based on the weight of the composition.

In embodiments, the composition may further comprise an additive selected from suspending agents, dispersing agents, antioxidants, buffers, pH adjusting agents, colorants, fragrances, preservatives and foam-stiffening agents.

In embodiments, the composition does not further comprise an additive.

In embodiments, the additive is an additional lipid. Thus, it may be that the composition comprises an additional lipid. It may be that the composition comprises one or more additional lipids. It may be that the composition further comprises up to 10% of an additional lipid, wherein the % is % by weight based on the weight of the composition. Thus, it may be that the composition comprises from 0.1% to 10% of an additional lipid, wherein the % is % by weight based on the weight of the composition. It may be that the composition comprises 0.1%, 0.25%, 0.5%, 0.75%, 1%, 2%, 3%, 4%, 5%, 6%, 7%, 8%, 9% or 10% of the additional lipid, wherein the % is % by weight based on the weight of the composition. It may be that the composition comprises no more than 10% of an additional lipid. It may be that the composition does not comprise an additional lipid, i.e. is substantially free of an additional lipid.

### Uses

Also provided herein, is the use of a formulation comprising more than 10% w/w to 30% w/w water, and 70% w/w to 90% w/w lipid, as a carrier for a pharmaceutically active agent, wherein the lipid is selected from monolinolein or monoolein. In embodiments, the formulation comprises more than 10% w/w to 25% w/w water, and 75% w/w to 90% w/w lipid, as a carrier for a pharmaceutically active agent, wherein the lipid is selected from monolinolein or monoolein. In embodiments, it may be that the pharmaceutically active agent is dispersed or dissolved in the carrier.

The formulation furthermore provides sustained and controlled release of the pharmaceutically active agent with a tuneable window of duration. The formulation is therefore highly suitable for the formation of depot compositions following non-parenteral (e.g. topical) administration to body cavities and/or surfaces of the body and are formed from lipids which may provide inherent benefits in themselves in addition to forming highly effective carriers and topical depots for active agents.

Thus, in embodiments, the carrier provides controlled release of the pharmaceutically active agent at a temperature of 36° C. to 39° C. It may be that the carrier provides controlled release of the pharmaceutically active agent at a temperature of 36° C. to 39° C., 37° C. to 39° C., or 37.5° C. to 38.5° C. For example, it may be that the carrier provides controlled release of the pharmaceutically active agent at a temperature of 36.0° C., 36.1° C., 36.2° C., 36.3° C., 36.4° C., 36.5° C., 36.6° C., 36.7° C., 36.8° C., 36.9° C., 37.0° C., 37.1° C., 37.2° C., 37.3° C., 37.4° C., 37.5° C., 37.6° C., 37.7° C., 37.8° C., 37.9° C., 38.0° C., 38.1° C., 38.2° C., 38.3° C., 38.4° C., 38.5° C., 38.6° C., 38.7° C., 38.8° C., 38.9° C., or 39.0° C. Preferably, it may be that the carrier provides controlled release of the pharmaceutically active agent at a temperature of 38° C.

In embodiments, the carrier forms a controlled release depot for the pharmaceutically active agent at a temperature of 36° C. to 39° C. It may be that the carrier forms a controlled release depot for the pharmaceutically active agent at a temperature of 36° C. to 39° C., 37° C. to 39° C., or 37.5° C. to 38.5° C. For example, it may be that the carrier forms a controlled release depot for the pharmaceutically active agent at a temperature of 36.0° C., 36.1° C., 36.2° C., 36.3° C., 36.4° C., 36.5° C., 36.6° C., 36.7° C., 36.8° C., 36.9° C., 37.0° C., 37.1° C., 37.2° C., 37.3° C., 37.4° C., 37.5° C., 37.6° C., 37.7° C., 37.8° C., 37.9° C., 38.0° C., 38.1° C., 38.2° C., 38.3° C., 38.4° C., 38.5° C., 38.6° C., 38.7° C., 38.8° C., 38.9° C., or 39.0° C. Preferably, it may be that the carrier forms a controlled release depot for the pharmaceutically active agent at a temperature of 38° C.

Thus, an advantage of the controlled release depots of the present invention is that the pharmaceutically active agents are released gradually over long periods without the need for repeat dosing.

The formulations of the present invention may form non-parenteral depots where the pharmaceutically active agent is slowly released at a body surface. It is particularly significant that the compositions generated from the formulations are bioadhesive because this allows local release of the pharmaceutically active agent over a sustained period. Thus, the compositions should coat the surface to which they are applied and should remain in place even when this surface is subjected to a flow of air or liquid and/or rubbing. For example, it may be that the compositions are administered rectally, and coat the wall of the colon, where the composition remains in place for the desired retention period (as described herein). It may be that the compositions are administered rectally, and coat the wall of the sigmoid colon, descending colon, and/or rectum, where the composition remains in place for the desired retention period (as described herein).

Suitably, the carrier is administered to a body cavity. For example, the formulation is a rectal formulation. Thus, in preferred embodiments, the carrier is administered as an enema.

Also provided herein, is the use of a pre-formulation composition comprising a lipid and a pharmaceutically active agent for the manufacture of a composition of the invention, wherein the lipid is selected from monolinolein or monoolein.

Also provided is the use of a pre-formulation composition comprising a monoacylglycerol lipid and a pharmaceutically active agent for the manufacture of a composition of the invention. It may be that the monoacylglycerol lipid comprises at least 50% by weight monolinolein.

In embodiments, the pharmaceutically active agent in the pre-formulation is a hydrophilic pharmaceutically active agent, as described herein.

In embodiments, the pre-formulation composition is a lyophilised mixture. In embodiments, the lyophilised mixture is hydrated with water to provide the composition of the invention.

### Therapeutic Uses and Applications

The composition of the invention may advantageously be used for rectal delivery of pharmaceutically active agents, by forming a sustained release depot in situ.

The compositions of the invention include modified release compositions which comprise monoacylglycerol lipids (such as monolinolein (MLO)), water, and a pharmaceutically acceptable agent, to target release of the pharmaceutically acceptable agent to the lower GI tract (GIT), particularly in the colon and/or rectum.

Accordingly, the compositions according to the invention comprising a pharmaceutically acceptable agent for local treatment of the lower GIT are expected to be useful in the treatment or prevention of a condition of the GIT. In certain embodiments the composition of the invention is for use in the treatment or prevention of a condition affecting the descending colon, sigmoid colon and/or the rectum. In certain embodiments the composition of the invention is for use in the treatment or prevention of a condition affecting the rectum. In certain embodiments the composition of the invention is for use in the treatment or prevention of a condition affecting sigmoid colon. In certain embodiments the composition of the invention is for use in the treatment or prevention of a condition affecting the descending colon. It may be that the pharmaceutically acceptable agent is a hydrophilic pharmaceutically acceptable agent, such as tofacitinib (TOFA). It may be that the pharmaceutically acceptable agent is a hydrophobic pharmaceutically acceptable agent, such as tacrolimus (TAC). It may be that the composition comprises one or more pharmaceutically acceptable agents. Thus, it may be that the composition comprises TOFA and a further pharmaceutically acceptable agent. It may be that the composition comprises TAC and a further pharmaceutically acceptable agent. It may be that the composition comprises mesalamine and a further pharmaceutically acceptable agent. It may be that the composition comprises budesonide and a further pharmaceutically acceptable agent. For example, the composition of the invention may comprise TOFA and/or a further pharmaceutically acceptable agent and be useful in the prevention or treatment of inflammatory conditions affecting the lower GI tract, particularly conditions affecting the colon. The composition of the invention may comprise TAC and/or a further pharmaceutically acceptable agent and be useful in the prevention or treatment of inflammatory conditions affecting the lower GI tract, particularly conditions affecting the colon. It may be that the composition of the invention comprises mesalamine and/or a further pharmaceutically acceptable agent and be useful in the prevention or treatment of inflammatory conditions affecting the lower GI tract, particularly conditions affecting the colon. It may be that the composition of the invention comprises budesonide and/or a further pharmaceutically acceptable agent and be useful in the prevention or treatment of inflammatory conditions affecting the lower GI tract, particularly conditions affecting the colon.

In embodiments, the composition of the invention may be administered via injection, for example, as a subcutaneous, intramuscular or intradermal injectable formulation, preferably a subcutaneous injectable formulation.

In other embodiments, the composition of the invention may be used for vaginal delivery of pharmaceutically active agents, by forming a sustained release depot in situ. In embodiments, the composition of the invention may be applied topically, e.g. via rectal administration or vaginal administration. Thus, it may be that the composition is topically applied to the colon, for example, as an enema. The dose required will vary depending upon the specific condition being treated and the stage of the condition. In the case of compositions containing TAC, the composition will generally be administered to provide a dose of TAC of from 0.1 mg to 5 mg, for example a dose of from 0.1 mg to 3 mg, or particularly a dose of 0.5 mg to 1.5 mg of TAC. In the case of compositions containing TOFA, the composition will generally be administered to provide a dose of TOFA of from 0.1 mg to 10 mg, for example a dose of from 2.5 mg to 10 mg, or particularly a dose of 5 mg to 10 mg of TOFA. The composition is suitably administered as a single daily or bi-daily dose, preferably a bi-daily dose. Alternatively, the composition is administered as a once-weekly dose.

In one aspect of the invention there is provided a composition of the invention for use in the treatment or prophylaxis of a disease. In embodiments, the composition of the invention for use in inhibiting or preventing disease progression. It may be that the disease is selected from the group consisting of: an inflammatory bowel disease, Crohn's disease, ulcerative colitis, irritable bowel syndrome (e.g. with constipation, diarrhoea and/or pain symptoms), diverticulosis, diverticulitis, proctitis, chemotherapy-associated enteritis, radiation-associated enteritis, colitis, colorectal carcinoma, adenocarcinoma, inflammatory disorders such as diversion colitis, ischemic colitis, infectious colitis, chemical colitis, microscopic colitis (including collagenous colitis and lymphocytic colitis), atypical colitis, pseudomembraneous colitis, fulminant colitis, autistic enterocolitis, indeterminate colitis, ileocolitis, granulomatous colitis, familial adenomatous polyposis, or perinanal Crohn's, including perianal fistulae.

Thus, in embodiments, the composition of the invention for use in the treatment or prophylaxis of an inflammatory bowel disease, Crohn's disease, ulcerative colitis, irritable bowel syndrome (e.g. with constipation, diarrhoea and/or pain symptoms), diverticulosis, diverticulitis, proctitis, chemotherapy-associated enteritis, radiation-associated enteritis, colitis, colorectal carcinoma, adenocarcinoma, inflammatory disorders such as diversion colitis, ischemic colitis, infectious colitis, chemical colitis, microscopic colitis (including collagenous colitis and lymphocytic colitis), atypical colitis, pseudomembraneous colitis, fulminant colitis, autistic enterocolitis, indeterminate colitis, ileocolitis, granulomatous colitis, familial adenomatous polyposis, or perinanal Crohn's, including perianal fistulae.

In one embodiment, the composition of the invention is for use in the treatment of an inflammatory bowel disease. In embodiments, the composition of the invention is for use in inhibiting or preventing progression of an inflammatory bowel disease. The main forms of inflammatory bowel disease are Crohn's disease and ulcerative colitis. Accordingly, the composition of the invention may be useful in the treatment of both of these conditions.

The composition of the invention may be for use in the treatment or prevention of irritable bowel syndrome (e.g. with constipation, diarrhoea and/or pain symptoms), diverticulitis, proctitis, radiation-associated enteritis, colitis, diverticulosis, colorectal carcinoma, adenocarcinoma, inflammatory disorders such as diversion colitis, ischemic colitis, infectious colitis, chemical colitis, microscopic colitis (including collagenous colitis and lymphocytic colitis), atypical colitis, pseudomembraneous colitis, fulminant colitis, autistic enterocolitis, indeterminate colitis, ileocolitis, or granulomatous colitis. The composition may also be for use in the treatment or prevention of Clostridium difficile colitis.

Crohn's disease may affect the entire GI tract including the colon. However, ulcerative colitis is a condition which affects only the colon and the rectum. Accordingly, the release profile provided by the colon-targeted, pharmaceutically acceptable agent-containing (e.g. TAC-containing or TOFA-containing) composition according to the invention is expected to be especially beneficial in the treatment of ulcerative colitis.

The colon-targeted, composition of the invention primarily releases the pharmaceutically acceptable agent (e.g. TAC or TOFA), in the colon. However, the pharmaceutically acceptable agent may also be released higher in the GI tract and accordingly the composition may also provide therapeutic benefit in conditions which affect other parts of the lower GI tract, for example Crohn's disease, irritable bowel syndrome (e.g. with constipation, diarrhoea and/or pain symptoms), diverticulitis, collagenous colitis, proctitis, radiation-associated enteritis, diverticulosis, colorectal carcinoma, or adenocarcinoma.

In a further embodiment there is provided a composition of the invention for use in the treatment or prophylaxis of ulcerative colitis. In embodiments, the composition of the invention is for use in inhibiting or preventing progression of ulcerative colitis.

A further aspect of the invention provides a composition comprising a pharmaceutically active agent as defined herein for use in the treatment of a cancer affecting the GI tract, particularly the lower GI tract, and especially the colon. Accordingly, the composition comprising the pharmaceutically active agent may be for use in the treatment of colorectal cancer. The composition comprising the pharmaceutically active agent may be for use in providing a cytostatic effect in a cancer affecting the GI tract, particularly a colorectal cancer.

Also provided is a composition comprising a pharmaceutically active agent for use in the preventing or delaying the onset of a cancer of the GI tract in a patient with chronic inflammatory condition affecting the GI tract, particularly the lower GI tract and especially the colon. For example, the composition comprising the pharmaceutically active agent may be for use in inhibiting tumourigenesis in the GI tract, particularly the colon.

The composition comprising the pharmaceutically active agent may be used alone or together with another anti-cancer agents to treat or delay the onset of a cancer affecting the GI tract. Thus, in embodiments, the pharmaceutically active agent in the composition may be an anticancer agent. Alternatively, the composition comprising the pharmaceutically active agent may be administered to a subject as a fixed dose combination with one or more additional anticancer agents. Anti-cancer agents which may be suitable for use with the composition are described herein.

Also provided is a composition comprising a pharmaceutically active agent for use in the prevention or treatment of fibrotic diseases or disorders. It may be that the fibrotic disease or disorder is selected from the group consisting of intestinal fibrosis, intra articular fibrosis, vaginal fibrosis, arthrofibrosis, endometrial fibrosis, endometriosis, epidural fibrosis, and skin fibrosis.

Also provided is a composition comprising a pharmaceutically active agent for use in the prevention or treatment of a fungal infection, for example, a vaginal fungal infection, or a fungal colonic infection (e.g. Paracoccidioidomycosis, histoplasmosis, and candidiasis).

Also provided is a composition comprising a pharmaceutically active agent for use in the prevention or treatment of a bacterial infection, for example, a bacterial infection caused by Crohn's, e.g. fistulas and/or abscesses.

### Ulcerative Colitis

Ulcerative colitis (UC) is a chronic inflammatory disease characterised by diffuse mucosal inflammation of the colon. The disease is characterised by amongst other features bloody diarrhoea, often with symptoms of rectal urgency and tenesmus. The term “ulcerative colitis” used herein incudes diverticulitis, pouchitis, proctitis, diversion colitis, ischemic colitis, infectious colitis, chemical colitis, radiation-induced colitis, microscopic colitis (including collagenous colitis and lymphocytic colitis), atypical colitis, pseudomembraneous colitis, fulminant colitis, autistic enterocolitis, indeterminate colitis, and granulomatous colitis. The invention contemplates the use of the composition as described herein for the treatment of any of such condition. Also contemplated is the composition for use in the treatment of colitis associated with inflammatory diseases of the gastrointestinal tract, particularly colitis associated with inflammatory diseases affecting the colon.

When UC is suspected in a patient, initial diagnosis generally includes a complete blood count to check for anaemia, urinalysis, stool culture, erythrocyte sedimentation rate (ESR) as an indicator of inflammation, liver and renal function tests, and electrolyte studies. However, these markers alone may not be sufficient to definitively diagnose ulcerative colitis. Suitably, therefore, endoscopy is generally the most accurate diagnostic tool for UC. A flexible sigmoidoscopy is usually sufficient to diagnose UC, however, a full colonoscopy may be performed if diagnosis is unclear. This procedure involves an investigation for the presence of superficial ulceration, erythema or friability of the mucosa, loss of vascular appearance of the colon, and pseudopolyps.

Biopsies may also be taken in order to differentiate UC from Crohn's disease. The biopsy samples are generally taken at the time of endoscopy and are examined for distortion of crypt architecture, inflammation of the crypts, crypt abscesses, and haemorrhage or inflammation in the lamina propia.

The ulcerative colitis may affect part of the colon, or substantially the entire colon. The ulcerative colitis may be ulcerative proctosigmoiditis. Reference herein to “ulcerative proctosigmoiditis” refers to ulcerative colitis limited to the rectum and sigmoid colon.

The ulcerative colitis may be left-sided ulcerative colitis. Reference to “left-sided colitis” herein means ulcerative colitis which is limited to the proportion of the colon distal to the splenic flexure, more particularly ulcerative colitis that extends beyond the rectum and as far proximally as the splenic flexure.

The ulcerative colitis may be extensive ulcerative colitis wherein substantially all of the colon is affected. Reference to “extensive ulcerative” or “pancolitis” herein means ulcerative colitis which extends proximal to the splenic flexure (i.e. extending beyond the splenic flexure towards the ileo-caecal junction).

Accordingly, the composition of the invention comprises a pharmaceutically active agent for use in the treatment of ulcerative colitis that affects any part or substantially the whole of the colon, for example, ulcerative colitis selected from ulcerative proctosigmoiditis, left-sided ulcerative colitis and extensive ulcerative colitis.

Ulcerative colitis is generally further characterised by the severity of the disease and may be mild, moderate or severe ulcerative colitis. Accordingly, the composition of the invention comprises a pharmaceutically active agent for use in the treatment of mild, moderate or severe ulcerative colitis. For example, the use of the composition in accordance with the invention may be for use in the treatment of mild ulcerative colitis. The use of the composition in accordance with the invention may be for use in the treatment of moderate ulcerative colitis. The use of the composition in accordance with the invention may be for use in the treatment of severe ulcerative colitis. The use of the composition in accordance with the invention may be for use in the treatment of patients with mild or moderate ulcerative colitis. The use of the composition in accordance with the invention may be for use in the treatment of patients with moderate or severe ulcerative colitis.

The severity of the ulcerative colitis may be determined by known methods, which generally rely upon a combination of patient characteristics. For example mild, moderate or severe UC may be determined as described in Dignas et al., “Second European evidence-based consensus on the diagnosis and management of ulcerative colitis: Definitions and diagnosis”, J. Crohns Colitis. 2012 December; 6 (10), which is incorporated herein by reference. Mild, moderate and severe ulcerative colitis may also be defined according to the criteria adopted by Truelove and Witts; Cortisone in ulcerative colitis; final report on a therapeutic trial. Br Med J 1955; 2:1041-8.

It is to be understood that methods of treatment corresponding to any of the uses of the composition in the treatment of ulcerative colitis described herein are intended to be encompassed within the invention. Similarly, any of the uses described herein may be described in relation to the use of the composition in the manufacture of a medicament for use in any of the treatments of ulcerative colitis described herein. The invention encompasses all such corresponding uses in the manufacture of a medicament.

### Dosage and Dosage Regimens

The amount of pharmaceutically active agent to be formulated with the compositions of the present invention will depend upon the functional dose and the period during which the depot composition formed upon administration is to provide sustained release. Typically, the dose formulated for a particular pharmaceutically active agent will be around the equivalent of the normal single dose multiplied by the number times greater the expected duration of action the formulation is to provide. Evidently, this amount will need to be tailored to take into account any adverse effects of a large dose at the beginning of treatment and so this will generally be the maximum dose used. The precise amount suitable in any case will readily be determined by suitable experimentation.

It may be that the composition of the invention is administered topically, for example administration for a period of less than 2 weeks.

The duration of treatment will depend upon the nature of the infection being treated. Suitably, the topical administration is continued until the condition is eradicated and/or the symptoms of the condition are reduced or eliminated. The upper limit of the period of treatment can be readily determined by a physician. The composition may, for example, be topically administered for a period selected from 1 day, 2 days, more than 3 days, more than 1 week, more than 2 weeks, more than 3 weeks, more than 4 weeks, more than 6 weeks, more than 12 weeks, more than 6 months and more than 1 year. For example, the composition may be topically administered for a period of more than two weeks to about 1 year; a period 3 weeks to 1 year; a period of 4 weeks to 1 year; a period of 4 weeks to 6 months; or 4 weeks to 3 months.

The frequency of administration of the composition of the invention will depend upon a number of factors that may readily be determined by a physician, for example the severity of the condition, the responsiveness to initial treatment and the particular condition being treated. Suitably, the composition of the invention may be topically administered once per day, twice per day, three times per day, four times per day, once every other day or once per week. Preferably, the composition of the invention may be topically applied to the colon of a subject. Suitably, the composition may be administered as an enema.

The dosage of the pharmaceutically active agent administered with the composition of the invention will vary depending upon a number of factors including, for example the age, weight and gender of animal or human suffering from the condition, the severity of the condition and the selected administration frequency.

A suitable dosage for topical application can be readily determined by a physician. The composition suitably comprises the pharmaceutically active agent in an amount of from about 0.1% to about 20%, preferably from about 0.1% to about 10%, more preferably from about 0.5% to about 6%, and even more preferably from about 1% to about 5%, wherein the % is % by weight based on the weight of the composition.

The composition of the invention is suitably topically applied to the colon of a subject. Preferably, the composition of the invention is administered rectally, for example, the composition of the invention is administered as an enema.

### Routes of Administration

The composition of the invention may be administered to a subject by any suitable route of administration, appropriate for the condition to be treated and the pharmaceutically active agent to be used. For example, topically administered, rectally administered, vaginally administered, or intravenously administered. It may be that the composition is topically administered, rectally administered, or vaginally administered.

Routes of administration include, but are not limited to, oral (e.g., by ingestion, tablets, sprays etc.); buccal; sublingual; transdermal (including, e.g., by a patch, plaster, dressing etc.); transmucosal (including, e.g., by a patch, plaster, etc.); ocular (e.g., by eyedrops); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; or by implant.

It may be that the composition of the invention is a non-parental composition (e.g. topical), and is administered to the surface of skin, mucous membranes and/or nails, to opthalmological, nasal, oral or internal surfaces or to cavities such as nasal, rectal, vaginal or buccal cavities, the periodontal pocket or cavities formed following extraction of a natural or implanted structure or prior to insertion of an implant (e.g a joint, stent, cosmetic implant, tooth, tooth filling or other implant).

In preferred embodiments, the composition of the invention is administered rectally. Thus, it may be that the composition of the invention is in the form of a suppository; a rectal capsule; a rectal solution, emulsion or suspension; a powder or tablet for rectal solutions or suspensions; a semi-solid rectal preparation; a rectal foam; a rectal tampon; or an enema. In preferred embodiments, it may be that the composition is administered to the subject via the rectum in the form of: a suppository; a rectal capsule; a semi-solid rectal preparation; a rectal foam; a rectal tampon; or an enema. Preferably, it may be that the composition of the invention is administered rectally as an enema. Thus, in a preferred embodiment, the composition of the invention is administered directly as a flowable composition. For example, the composition of the invention is administered rectally as a lamellar gel, e.g. in the form of an enema.

As described herein, at room temperature (e.g. at about 25° C.), the composition of the invention has a lamellar phase structure (e.g. the composition of the invention is a lamellar gel). Accordingly, the compositions of the invention are easily administered into the lower gastrointestinal (GI) tract rectally, and quickly convert into the highly viscous lipid cubic phase at rectal temperature (e.g. a temperature of from about 36° C. to 39° C.). This is particularly advantageous as thick, viscous preparations can be difficult to apply effectively rectally to the lower GI tract. Moreover, less viscous preparations (although easy to administer) are poorly retained in situ which results in rapid loss of material. In contrast, the compositions of the invention act as a highly viscous bioadhesive controlled depot system, and are retained in situ for at least about 6 hours.

It is well understood that the lower GI tract begins at the cecum and also includes the appendix (in humans), colon (e.g. sigmoid colon, descending colon, transverse colon, ascending colon), splenic flexure, hepatic flexure, rectum, and anus. As described herein, the composition of the invention is administered to the lower GI tract of a subject. Thus, it may be that the composition of the invention is topically applied to the lining of the lower GI tract. For example, it may be that the composition of the invention is topically applied to the lining of the colon and/or the rectum. Thus, it may be that the composition of the invention is topically applied to the lining of the lower GI tract via the rectum, e.g. in the form of an enema. For example, it may be that the composition of the invention is topically applied to the lining of the colon via the rectum, e.g. in the form of an enema. In particular embodiments, the composition of the invention is topically applied to the rectum, sigmoid colon, and/or the descending colon. More preferably, the composition of the invention is topically applied to the rectum and/or sigmoid colon.

Enema devices and/or kits used for enema delivery are well known, and include, for example, an enema bag, tubing, nozzles, syringes (such as rectal bulb syringes), etc. In embodiments, the composition of the invention is delivered locally via an endoscope for targeting of the composition to specific areas of the lower GI tract, e.g., sites of inflammation, damage, tumours, polyps etc. It may be that the composition of the invention is delivered via an endoscope to the tissues of the sigmoid colon, descending colon, transverse colon, ascending colon and/or rectum. In embodiments, the composition of the invention is administered rectally via a rectal catheter.

It may be that the subject has had a colostomy (a surgical procedure to connect the colon through an opening in the abdominal wall (stoma)). Thus, it may be that the subject has a stoma. Suitably, the composition of the invention may be administered to the lower GI tract of the subject via the stoma, e.g. in the form of a suppository or an enema. It may be that the composition of the invention is administered to the lower GI tract of the subject via the stoma, in the form of an enema. For example, it may be that the composition of the invention may be administered to the colon of the subject via the stoma, in the form of an enema. Thus, it may be that the composition of the invention is topically applied to the lining of the lower GI tract via the stoma, in the form of an enema. For example, it may be that the composition of the invention is topically applied to the lining of the colon via the stoma, in the form of an enema.

In embodiments, the composition of the invention is a parental composition. Thus, it may be that the composition of the invention is an injectable formulation and is administered to the subject subcutaneously, intramuscularly or intradermally. Preferably, the composition is administered to the subject subcutaneously.

In embodiments, the composition of the invention is administered vaginally. Thus, it may be that the composition of the invention is in the form of a vaginal tablet; a vaginal suppository or pessary; a vaginal foam, spray, gel, or cream.

### Subject

The compositions of the invention are suitable for use in the treatment of a subject affected by any of the diseases or conditions described herein. Preferably, the compositions of the invention are suitable for use in the topical treatment of a subject affected by any of the diseases or conditions described herein, wherein the composition is applied topically to the colon of a subject, e.g, wherein the composition is administered rectally to a subject, preferably as an enema.

In embodiments, the subject may be a warm-blooded mammal. In particular embodiments, the subject treated is a human. It may be that the subject is an adult human (aged 18 years or more). It may be that the subject is a paediatric human aged less than 18 years. It may be that the paediatric subject is aged from 2 to 4 years. It may be that the paediatric subject is aged from 5 to 10 years. It may be that the paediatric subject is aged from 11 to 18 years.

In embodiments, the subject may be an animal. In certain embodiments, the composition of the invention is for use as veterinary product for the topical treatment of an animal. In certain embodiments, the compositions of the invention are for use in the topical treatment of diseases and conditions in commercial animals such as livestock (e.g. cows, sheep, chickens, pigs, geese, ducks, goats, etc.). In other embodiments, the compositions of the present invention may be for use in the topical treatment of diseases or conditions in companion animals such as cats, dogs, horses, etc.

### Method of Preparation

The composition of the invention may be prepared by a method comprising the steps:
a) hydrating a mixture comprising a lipid and a pharmaceutically active agent with water, to provide a lipid-drug mixture; and
b) equilibrating the lipid-drug mixture to provide the composition.

In embodiments, the pharmaceutically active agent is a hydrophobic pharmaceutically active agent. For example, it may be that the pharmaceutically active agent is tacrolimus. Thus, it may be that the composition is prepared by a method comprising the steps:
a) hydrating a mixture comprising a lipid and a pharmaceutically active agent (e.g. a hydrophobic pharmaceutically active agent) with water, to provide a lipid-drug mixture; and
b) equilibrating the lipid-drug mixture to provide the composition.

In embodiments, the lipid is a monoacylglycerol lipid. In embodiments, the monoacylglycerol lipid is selected from monolinolein or monoolein. It may be that the lipid is monolinolein. It may be that the lipid is monoolein.

In embodiments, the lipid is a monoacylglycerol lipid that comprises monolinolein or monoolein, or combinations thereof. In embodiments, the lipid is a monoacylglycerol lipid that comprises at least 50% by weight monolinolein.

Thus, in embodiments, the composition of the invention may be prepared by a method comprising the steps:
a) hydrating a mixture comprising a lipid and a pharmaceutically active agent (e.g. a hydrophobic pharmaceutically active agent) with water, to provide a lipid-drug mixture; and
equilibrating the lipid-drug mixture to provide the composition, b)
   wherein the lipid is selected from monolinolein or monoolein. Preferably, the lipid is monolinolein.

In embodiments, in step a) the mixture is hydrated with deionised water. In embodiments, in step a) the mixture is hydrated with phosphate buffered saline (PBS). In embodiments, in step a) the mixture is hydrated with water for injection (WFI).

In embodiments, the lipid-drug mixture of step a) is vortexed at room temperature until a homogenous mixture is obtained.

In embodiments, in step b), the lipid-drug mixture is equilibrated up to about 48 h. It may be that the lipid-drug mixture is equilibrated for about 1 min, 2 min, 3 min, 4 min, 5 min, 10 min, 20 min, 30 min, 40 min, 50 min, 1 h, 2 h, 3 h, 4 h, 5 h, 6 h, 7 h, 8 h, 9 h, 10 h, 11 h, 12 h, 18 h, 24 h, 30 h, 36 h, 42 h, or 48 h.

In embodiments, the pharmaceutically active agent is photosensitive. Thus, it may be that the lipid-drug mixture in step b) is equilibrated under dark conditions.

In embodiments, the mixture in step a) is a lyophilised mixture. It may be that the lyophilised mixture is obtained by:
i) dissolving the lipid and the pharmaceutically active agent in an organic solvent; and
ii) lyophilising the mixture of i) to provide the lyophilised mixture.

In embodiments, in step i), it may be that the organic solvent is an alcohol. Examples of suitable alcohols include ethanol, methanol, isopropanol and glycerol formal. It may be that the organic solvent is ethanol. It may be that the organic solvent is methanol. Preferably, the organic solvent is ethanol.

In embodiments, in step ii) the mixture is lyophilised by removing the organic solvent. It may be that the organic solvent is removed under reduced pressure. It may be that the organic solvent is removed by freeze-drying the mixture of i). It may be that the mixture of i) is freeze-dried for about 24 h. Suitably, the mixture of i) is freeze-dried for 24 h at 0.22 mbar to provide the lyophilised mixture.

In other embodiments, the pharmaceutically active agent is a hydrophilic pharmaceutically active agent. It may be that the pharmaceutically active agent is a Janus kinase inhibitor. Preferably, it may be that the pharmaceutically active agent is tofacitinib, or a pharmaceutically acceptable salt thereof. Thus, the composition of the invention may also be prepared by a method comprising the steps:
a) dissolving a pharmaceutically active agent (e.g. a hydrophilic pharmaceutically active agent) in water to provide a drug mixture;
b) hydrating a lipid with the drug mixture, to provide a lipid-drug mixture; and
c) equilibrating the lipid-drug mixture to provide the composition.

In embodiments, the lipid is a monoacylglycerol lipid. In embodiments, the monoacylglycerol lipid is selected from monolinolein or monoolein. It may be that the lipid is monolinolein. It may be that the lipid is monoolein.

In embodiments, the lipid is a monoacylglycerol lipid that comprises monolinolein or monoolein, or combinations thereof. In embodiments, the lipid is a monoacylglycerol lipid that comprises at least 50% by weight monolinolein.

Thus, in embodiments, the composition of the invention may also be prepared by a method comprising the steps:
a) dissolving a pharmaceutically active agent (e.g. a hydrophilic pharmaceutically active agent) in water to provide a drug mixture;
b) hydrating a lipid with the drug mixture, to provide a lipid-drug mixture; and
c) equilibrating the lipid-drug mixture to provide the composition,
   wherein the lipid is selected from monolinolein or monoolein. Preferably, the lipid is monolinolein.

In embodiments, in step a), the pharmaceutically active agent is dissolved in deionised water. In embodiments, in step a) the pharmaceutically active agent is dissolved in water for injection (WFI).

In embodiments, the lipid-drug mixture of step b) is vortexed at room temperature until a homogenous mixture is obtained. It may be that the lipid-drug mixture of step b) is vortexed at room temperature for greater than 30 s to at least 10 min. It may be that the lipid-drug mixture of step b) is vortexed at room temperature for at least 30 s, 40 s, 50 s, 1 min, 2 min, 3 min, 4 min, 5 min, 6 min, 7 min, 8 min, 9 min, or 10 min. Suitably, it may be that the lipid-drug mixture of step b) is vortexed at room temperature for at least 5 min.

In embodiments, in step c), the lipid-drug mixture is equilibrated up to about 48 h. It may be that the lipid-drug mixture is equilibrated for about 1 min, 2 min, 3 min, 4 min, 5 min, 10 min, 20 min, 30 min, 40 min, 50 min, 1 h, 2 h, 3 h, 4 h, 5 h, 6 h, 7 h, 8 h, 9 h, 10 h, 11 h, 12 h, 18 h, 24 h, 30 h, 36 h, 42 h, or 48 h.

In embodiments, the pharmaceutically active agent is photosensitive. Thus, it may be that the lipid-drug mixture in step c) is equilibrated under dark conditions.

The composition of the invention may also be prepared by a method comprising the following steps:
a) heating the lipid to provide a molten lipid;
b) mixing the molten lipid with the pharmaceutically active agent, to provide a lipid-drug mixture;
c) mixing the lipid-drug mixture with water; and
d) equilibrating the lipid-drug mixture and water to provide the composition.

In embodiments, the lipid is a monoacylglycerol lipid. In embodiments, the monoacylglycerol lipid is selected from monolinolein or monoolein. It may be that the lipid is monolinolein. It may be that the lipid is monoolein.

In embodiments, the lipid is a monoacylglycerol lipid that comprises monolinolein or monoolein, or combinations thereof. In embodiments, the lipid is a monoacylglycerol lipid that comprises at least 50% by weight monolinolein.

In embodiments, in step a) the lipid is heated to a temperature of about 30° C. to 70° C. It may be that the lipid is heated to a temperature of about 40° C. to 60° C. It may be that the lipid is heated to a temperature of about 45° C. to 55° C. Preferably, it may be that the lipid is heated to a temperature of about 50° C.

In embodiments, in step b) the molten lipid and pharmaceutically active agent are heated to a temperature of about 30° C. to 70° C. It may be that the molten lipid and pharmaceutically active agent are heated to a temperature of about 40° C. to 60° C. It may be that the molten lipid and pharmaceutically active agent are heated to a temperature of about 45° C. to 55° C. Preferably, it may be that the molten lipid and pharmaceutically active agent are heated to a temperature of about 50° C.

In embodiments, in step b) the molten lipid and pharmaceutically active agent are mixed until a homogenous lipid-drug mixture is obtained. It may be that the molten lipid and pharmaceutically active agent are mixed for up to about 1 h. It may be that the molten lipid and pharmaceutically active agent are mixed for up to about 30 min. It may be that the molten lipid and pharmaceutically active agent are mixed for up to about 15 min. It may be that the molten lipid and pharmaceutically active agent are mixed for up to about 5 min. It may be that the molten lipid and pharmaceutically active agent are mixed for about 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, 25 min, 30 min, 35 min, 40 min, 45 min, 50 min, 55 min, or 1 h.

In embodiments, in step c) the lipid-drug mixture is mixed with deionised water. In embodiments, in step c) the lipid-drug mixture is mixed with phosphate buffered saline (PBS). In embodiments, in step c) the lipid-drug mixture is mixed with water for injection (WFI).

The lipid-drug mixture in step c) can be mixed by any method known in the art. For example, it may be that the lipid-drug mixture is mixed with water in a dual-syringe. A dual syringe can comprise two separate chambers, a mixing nozzle and a plunger. Accordingly, the lipid-drug mixture is inserted into the first chamber, and the water is inserted into the second chamber. When force is applied to the plunger, the lipid-drug mixture and the water are mixed in the mixing nozzle, to obtain a homogenous mixture. Alternatively, a dual-syringe can comprise two syringes which are attached via a connector. Accordingly, the lipid-drug mixture is inserted into the first syringe, and the water is inserted into the second syringe. The lipid-drug mixture in the first syringe is transferred into the second syringe containing the water. Then the lipid-drug mixture and the water are transferred back into the first syringe. This process is repeated until a homogenous mixture is obtained. Thus, it may be that the lipid-drug mixture in step c) is mixed with a dual-syringe.

In embodiments, in step d), the lipid-drug mixture and water are equilibrated up to about 1 h. It may be that the lipid-drug mixture is equilibrated up to about 30 min. It may be that the lipid-drug mixture is equilibrated up to about 15 min. It may be that the lipid-drug mixture is equilibrated up to about 5 min. It may be that the lipid-drug mixture is equilibrated up to about 1 min. It may be that the lipid-drug mixture is equilibrated for about 1 min, 2 min, 3 min, 4 min, 5 min, 6 min, 7 min, 8 min, 9 min, min, 11 min, 12 min, 13 min, 14 min, 15 min, 16 min, 17 min, 18 min, 19 min, 20 min, 25 min, 30 min, 35 min, 40 min, 45 min, 50 min, 55 min, or 1 h.

### Kit

Provided herein is a kit comprising:
a) a first container comprising a lipid and a pharmaceutically active agent; and
b) instructions to combine a) with water to provide the composition of the invention.

In embodiments, the lipid is a monoacylglycerol lipid. In embodiments, the monoacylglycerol lipid is selected from monolinolein or monoolein. It may be that the lipid is monolinolein. It may be that the lipid is monoolein.

In embodiments, the lipid is a monoacylglycerol lipid that comprises monolinolein or monoolein, or combinations thereof. In embodiments, the lipid is a monoacylglycerol lipid that comprises at least 50% by weight monolinolein.

Thus, in embodiments, the kit comprises:
a) a first container comprising a lipid and a pharmaceutically active agent; and
b) instructions to combine a) with water to provide the composition of the invention,
   wherein the lipid is selected from monolinolein or monoolein. Preferably, the lipid is monolinolein.

In embodiments, the pharmaceutically active agent is selected from any of the pharmaceutically active agents as defined herein. Suitably, the pharmaceutically active agent is a hydrophobic pharmaceutically active agent. It may be that the pharmaceutically active agent is tacrolimus.

In embodiments, the kit further comprises a second container, wherein the second container comprises water. It may be that the water is deionised water. It may be that the water is water for injection (WFI). Therefore, it may be that the kit comprises:
a) a first container comprising a lipid and a pharmaceutically active agent;
b) a second container comprising water; and
c) instructions to combine a) with b) to provide the composition of the invention,
   wherein the lipid is selected from monolinolein or monoolein.

In embodiments, the lipid and the pharmaceutically active agent in the first container are provided as a lyophilised mixture. It may be that the lyophilised mixture is obtained by the methods described herein. Thus, it may be that the kit comprises instructions to hydrate the lyophilised mixture with a certain amount of water, in order to arrive at the composition of the invention. For example, it may be that the kit comprises:
a) a first container comprising a lyophilised mixture, wherein the lyophilised mixture comprises lipid and a pharmaceutically active agent;
b) a second container comprising water; and
c) instructions to combine a) with b) to provide the composition of the invention,
   wherein the lipid is selected from monolinolein or monoolein.

In preferred embodiments, it may be that the kit comprises:
a) a first container comprising a lyophilised mixture, wherein the lyophilised mixture comprises monolinolein and tacrolimus;
b) a second container comprising water; and
c) instructions to combine a) with b) to provide the composition of the invention,
   wherein the resulting composition comprises:
(i) 1% w/w of tacrolimus;
(ii) 83.16% w/w monolinolein; and
(iii) 15.84% w/w water.

In other embodiments, the pharmaceutically active agent is a hydrophilic pharmaceutically active agent. For example, it may be that the pharmaceutically active agent is tofacitinib, or a pharmaceutically acceptable salt thereof. Thus, also provided is a kit comprising:
a) a first container comprising a lipid; and
b) instructions to combine a) with a solution comprising a pharmaceutically active agent (e.g. a hydrophilic pharmaceutically active agent) dissolved in water to provide the composition of the invention.

In embodiments, the lipid is a monoacylglycerol lipid. In embodiments, the monoacylglycerol lipid is selected from monolinolein or monoolein. It may be that the lipid is monolinolein. It may be that the lipid is monoolein.

In embodiments, the lipid is a monoacylglycerol lipid that comprises monolinolein or monoolein, or combinations thereof. In embodiments, the lipid is a monoacylglycerol lipid that comprises at least 50% by weight monolinolein.

Thus, in embodiments, the kit comprises:
a) a first container comprising a lipid; and
b) instructions to combine a) with a solution comprising a pharmaceutically active agent (e.g. a hydrophilic pharmaceutically active agent) dissolved in water to provide the composition of the invention,
   wherein the lipid is selected from monolinolein or monoolein. Preferably, the lipid is monolinolein.

In embodiments, the kit further comprises a second container, wherein the second container comprises the pharmaceutically active agent dissolved in water. It may be that the water is deionised water. It may be that the water is water for injection (WFI). Therefore, it may be that the kit comprises:
a) a first container comprising a lipid;
b) a second container comprising a solution comprising a pharmaceutically active agent dissolved in water; and
c) instructions to combine a) with b) to provide the composition of the invention,
   wherein the lipid is selected from monolinolein or monoolein.

It may be that the kit comprises instructions to hydrate the lipid in the first container with a certain amount of solution in the second container, in order to arrive at the composition of the invention. For example, it may be that the kit comprises:
a) a first container comprising monolinolein;
b) a second container comprising a solution comprising tofacitinib, or a pharmaceutically acceptable salt thereof dissolved in water; and
c) instructions to combine a) with b) to provide the composition of the invention,
   wherein the resulting composition comprises:
(i) 5% w/w of tofacitinib, or a pharmaceutically acceptable salt thereof;
(ii) 79.8% w/w monolinolein; and
(iii) 15.2% w/w water.

The following examples are intended to illustrate, but not to limit, the invention in any manner, shape, or form, either explicitly or implicitly.

### EXAMPLES

Solvents, reagents and starting materials were purchased from commercial vendors and used as received unless otherwise described. All reactions were performed at room temperature unless otherwise stated. Starting materials were purchased from commercial sources or synthesised according to the methods described herein or using literature procedures.

### Abbreviations

DCs: dendritic cells
DSS: dextran sodium sulfate
h: hour(s)
H: hexagonal phase
H&E: hematoxilin and eosin
HEPES: 4-(2-hydroxyethyl)-1-piperazineethanesulfonic acid
HPLC: High Performance Liquid Chromatography
L: lamellar phase
LMP: lipidic mesophase
LVR: linear viscoelastic regime
min: minute(s)
MLO: monolinolein
PBS: phosphate-buffered saline
Q: cubic phase
SAXS: Small Angle X-ray Scattering
TAC: tacrolimus
TIF-Gel: temperature-triggered in situ forming adhesive lipid gel
TOFA: tofacitinib
UC: ulcerative colitis
WAXS: Wide Angle X-ray Scattering

### Materials

Monolinolein (MLO) was purchased by NU-Check Prep, Inc. (MN, USA). Ultrapure water of resistivity 18.2 MΩ·cm was produced by Barnstead Smart2pure (Thermo scientific) and used as the aqueous phase. Methanol, acetonitrile, and tetrahydrofuran were analytical grade supplied by Fisher Scientific (Schwerte, Germany). Ethanol absolute >99.5 wt % was obtained from VWR chemicals BDH (London, UK). Tofacitinib citrate (TOFA) was purchased by LC laboratories (Woburn, MA) and tacrolimus (TAC) was obtained from R&S Pharmchem Co., Ltd (Shangai, China). Clotrimazole and mesalamine were purchased from Merck (Darmstadt, Germany). Budesonide (97% purity) was obtained from Thermo Scientific (Pittsburgh, USA). The lipase from porcine pancreas and methyl cellulose (viscosity 25 cp) were obtained from Sigma Chemical Co. (St. Louis, USA). Caffeine (Ph. Eur. Quality) was purchased from Hanseler Swiss Pharma. HEPES salt was obtained from Carl Roth (Karlsruhe, Germany).

### Analytical Methods

### Small Angle X-Ray Scattering (SAXS)

SAXS measurements were used to determine the phase identity and symmetry of the produced LMPs. Measurements were performed on a Bruker AXS Micro, with a microfocused X-ray source, operating at voltage and filament current of 50 kV and 1000 μA, respectively. The Cu Kα radiation (λCu Kα=1.5418 Å) was collimated by a 2D Kratky collimator, and the data were collected by a 2D Pilatus 100K detector. The scattering vector Q=(4π/λ) sin θ, with 2θ being the scattering angle, was calibrated using silver behenate. Data were collected and azimuthally averaged using the Saxsgui software to yield 1D intensity vs. scattering vector Q, with a Q range from 0.001 to 0.5 Å⁻¹. For all measurements the samples were placed inside a stainless-steel cell between two thin replaceable mica sheets and sealed by an O-ring, with a sample volume of 10 μL and a thickness of ˜1 mm. Measurements were performed at 25° C., 30° C., 34° C., 36° C. and 38° C. Samples were equilibrated for 10 min before measurements whereas scattered intensity was collected over 30 min and over 60 min in case of lamellar phase. On the other hand, for the kinetic study, the sample was pre equilibrated at 25° C. and inserted in the sample holder kept at 38° C. and the scattered intensity collected over 5 min. To determine the structural parameters such as the size of the water channels, SAXS data information on the lattice were combined with the composition of the samples (R. Mezzenga, et al., Shear rheology of lyotropic liquid crystals: a case study, Langmuir 21, 3322-3333 (2005)).

### SAXS Spectra Acquired at Different Temperatures on Gels Containing 10% w/w TOFA and 10% w/w TAC

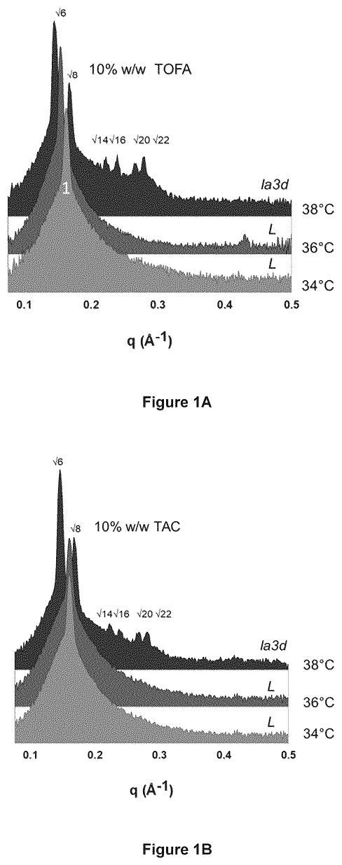

MLO was used as the lipid component of the mesophases and mixed with weighed amounts of drugs (10% w/w) in sealed Pyrex tubes and alternatively centrifuging (10 min, 5000 g) several times at room temperature until a homogenous mixture was obtained. The mesophase was then equilibrated for 48 h at room temperature in the dark (see, FIGS. 1A and 1B).

### Rheology Experiments

A stress-controlled rheometer (Modular Compact Rheometer MCR 72 from Anton Paar, Graz, Austria) was used in cone-plate geometry, 0.993° angle, and 49.942 mm diameter. The temperature control was set either at 25° C. or 38° C. First, a strain sweep was performed at 1 Hz between 0.002 and 100% strain to determine the linear range, the linear viscoelastic regime (LVR), the yield and low points. Then, oscillatory frequency sweeps were performed at 0.1% strain between 0.1 and 100 rad/s. Frequency sweep measurements were performed at a constant strain in the linear viscoelastic regime (LVR), as determined by the oscillation strain sweep (amplitude sweep) measurement performed for each sample. Within the linear viscoelastic region, in fact, the material response is independent of the magnitude of the deformation and the material structure is maintained intact; this is a necessary condition to accurately determine the mechanical properties of the material.

### Release Experiments of TAC and TOFA: In Vitro and Ex Vivo Set-Up and HPLC Drugs Quantification

Formulations and free drug enema were tested in vitro and ex vivo with vertical diffusion cells (PermeGear, Pennsylvania, USA) using a 3000 nm polycarbonate membrane (Sterlitech Corporation, USA). HEPES buffer with a pH 7.4 (8 mL) was used as the release medium and the device was placed in a shaking incubator at 100 rpm and 37° C. To investigate the effect of lipase on drug release, porcine pancreatic lipase (1000 U/mL) was added to the sample in the donor chamber. Ex vivo experiments were performed using rat intestinal tissue to evaluate the drug release of the TIF-Gel. Briefly, fresh intestinal tissue was obtained and cut into suitable samples (2 mm*1 mm*1 mm) for Franz cell apparatus. Tissue was placed on the polycarbonate membrane for tensile loading. At designated time points (0.5 h, 1 h, 1.5 h, 2 h, 3 h, 4 h, 5 h, 6 h, 7 h, 8 h), the release medium was completely replaced with 8 mL of fresh HEPES and 1 mL aliquot was taken for lyophilisation. Each sample was resuspended with internal standard in the mobile phase and the drug content was analysed by HPLC. The same experimental design was used for both TOFA- and TAC-loaded TIF-Gels. Moreover, samples of TIF-Gel containing drug were stored at room temperature and 4° C. for 30 days. The drug stability was determined by HPLC analysis. The same experimental design was used for both TOFA- and TAC-loaded TIF-Gels.

### HPLC Method: Tofacitinib Citrate

Tofacitinib citrate was detected by reverse-phase liquid chromatography using a Macherey-Nagel Nucleosil 100-5 C18 (4.0×250 mm; 5.0 μm particle size) column. The mobile phase consisted of acetonitrile/methanol/water (13:13:74 v/v)+0.1% trifluoroacetic acid at a flow rate of 1 mL/min, temperature 25° C. and UV detection at λ=278 nm. An internal standard (caffeine, 20 μg/mL) was added to each sample to correct for inter-injection variation and UV detection at λ=278 nm. Data were collected and analysed using the software Chromeleon 7 (Thermo Fisher).

### HPLC Method: Tacrolimus

Tacrolimus was detected by reverse-phase liquid chromatography using a Macherey-Nagel Nucleosil 100-5 C18 (4.0×250 mm; 5.0 μm particle size) column. The mobile phase consisted of methanol/water (80:20 v/v)+0.1% trifluoroacetic acid at a flow rate of 1 mL/min, temperature 50° C. and UV detection at λ=214 nm. An internal standard (ketoconazole, 20 μg/mL) was added to each sample to correct for inter-injection variation and UV detection at λ=278 nm. Data were collected and analysed using the software Chromeleon 7 (Thermo Fisher).

Release Experiments of Clotrimazole. Budesonide and Mesalamine: In Vitro Set-Up and HPLC Drugs Quantification

### Release Experiments: Clotrimazole

A 5% w/w clotrimazole formulation was prepared following the described preparation method in Example 1. Briefly, the TIF-Gel composition comprised: a) a carrier comprising: a1) water in an amount of 16% by weight of the carrier; and a2) MLO in an amount of 84% by weight of the carrier; and b) clotrimazole in an amount of 5% by weight of the composition. A 0.5 mg/ml solution of free clotrimazole was also prepared using Tween 80 2% w/v as solvent. The drug release profiles were then tested in vitro with vertical Franz cells (PermeGear, Pennsylvania, USA) and a 3000 nm polycarbonate membrane (Sterlitech Corporation, USA). The donor chamber contained 8 mL of Tween 80 2% w/v release medium. The loaded Franz cells were placed in a shaking incubator at 100 rpm and 37° C. For free clotrimazole and 5% w/w TIF-Gel, 300 UL and approximately 40 mg were placed directly in the donor chamber, respectively. At predetermined time points of 0.5, 1, 1.5, 2, 3, 4, 5, 6, 7, 8 h, the release medium was completely replaced, and an aliquot was lyophilised. Each sample was resuspended with a solution of acetonitrile/water (70:30 v/v) and the clotrimazole content was analysed by HPLC.

### HPLC Method: Clotrimazole

The clotrimazole concentration was determined by reverse-phase liquid chromatography using a Macherey-Nagel Nucleosil 100-5 C18 (4.0×250 mm; 5.0 μm particle size) column. The mobile phase consisted of acetonitrile/water (70:30 v/v)+0.1% trifluoroacetic acid at flow rate 0.5 mL/min, temperature 40° C. and UV detection at λ=200 nm.

### Release Experiments: Budesonide

A 5% w/w budesonide formulation was prepared following the described preparation method in Example 1. Briefly, the TIF-Gel composition comprised: a) a carrier comprising: a1) water in an amount of 16% by weight of the carrier; and a2) MLO in an amount of 84% by weight of the carrier; and b) budesonide in an amount of 5% by weight of the composition. A 0.5 mg/mL solution of free budesonide was also prepared using Tween 80 (2% w/v) as solvent. The drug release profiles were then tested in vitro with vertical Franz cells (PermeGear, Pennsylvania, USA) and a 3000 nm polycarbonate membrane (Sterlitech Corporation, USA). The donor chamber contained 8 mL of Tween 80 2% w/v release medium. The loaded Franz cells were placed in a shaking incubator at 100 rpm and 37° C. For free budesonide and 5% w/w TIF-Gel, 300 μL and approximately 40 mg were placed directly in the donor chamber respectively. At predetermined time points of 0.5, 1, 1.5, 2, 3, 4, 5, 6, 7, 8 h, the release medium was completely replaced, and an aliquot was lyophilised. Each sample was resuspended with a solution of acetonitrile/water (70:30 v/v) and the clotrimazole content was analysed by HPLC.

### HPLC Method: Budesonide

The budesonide concentration was determined by reverse-phase liquid chromatography using a Macherey-Nagel Nucleosil 100-5 C18 (4.0×250 mm; 5.0 μm particle size) column. The mobile phase consisted of acetonitrile/water (70:30 v/v)+0.1% trifluoroacetic acid at flow rate 0.5 mL/min, temperature 40° C. and UV detection at λ=260 nm.

### Release Experiments: Mesalamine

A 5% w/v mesalamine formulation was prepared following the described method in Example 1. Briefly, the TIF-Gel composition comprised: a) a carrier comprising: a1) water in an amount of 16% by weight of the carrier; and a2) MLO in an amount of 84% by weight of the carrier; and b) mesalamine in an amount of 5% by weight of the composition. A 1 mg/mL solution of free mesalamine in HEPES (10 mM, pH 7.4) was also prepared. The drug profile releases were tested in vitro with vertical Franz cells (PermeGear, Pennsylvania, USA) and a 3000 nm polycarbonate membrane (Sterlitech Corporation, USA). The donor chamber contained 8 mL of HEPES (10 mM, pH 7.4) release medium. The loaded Franz cells were placed in a shaking incubator at 100 rpm and 37° C. For free mesalamine and 5% w/w TIF-Gel, 300 μL and approximately 40 mg were placed directly in the donor chamber respectively. At predetermined time points of 0.5, 1, 1.5, 2, 3, 4, 5, 6, 7, 8 h, the release medium was completely replaced. Aliquots of 200 uL were directly analysed by absorbance.

### HPLC Method: Mesalamine

Mesalamine concentration was determined by absorbance using a microplate reader (Spark 10M, Tecan, Switzerland). The maximal absorbance wavelength of mesalamine was 298 nm for mesalamine in HEPES (10 mM, pH 7.4) at 20° C.

### Drug Homogeneity in the Gel Structure

To determine whether TOFA and TAC were homogeneously distributed into the gel matrix, the gel (loaded with TAC or TOFA) was prepared as described herein and transferred into a 2 mL Eppendorf tube. The tube was centrifuged and kept at rest for 24 hours. Subsequently, the gel was divided into 3 different layers (Top, Middle, and Bottom), and the drug content evaluated in each by HPLC.

### LC-MS/MS Analysis

Samples, standards and QC were extracted by protein precipitation and analysed by LC-MS/MS using the following method. For plasma samples, 10 μL of plasma was mixed with 25 μL of precipitation solution (80:20 Acetonitrile: Methanol+0.1 uM loperamide). The samples were centrifuged at 10000 g for 10 min and 20 μL of supernatant was diluted with 40 μL of H₂O+0.1% FA. The samples were centrifuged at 3400 rpm for 10 min and 50 μL of supernatant was diluted with 100 μL of H2O+0.1% FA. All the samples were analysed by LC-MS/MS (Shimadzu prominence HPLC coupled to an AB/SCIEX 4000 QTRAP) in positive MRM mode. The samples were separated on a Cortecs RP shield column (3×50 mm 2.6 u) using a fast gradient of 10 mM ammonium formate in water (A) and methanol (B). The gradient starts at 20% B and increases to 98% B in 2 minutes, hold for 0.5 min and equilibrates for 1.4 min. The MRM parameters were optimized for each analyte; the MRM transition 313. to 149.3 was selected for tofacitinib, 822.3 to 770.1 for tacrolimus and 477.1 to 266.0 for loperamide (internal standard). The samples were quantified using a calibration curve prepared in matrix using the area ration of analyte to internal standard.

### WAXS (Wide Angle X-Ray Scattering)

WAXS measurements were performed on a Bruker AXS Micro, with a microfocused X-ray source, operating at voltage and filament current of 50 kV and 1000 μA, respectively. The Cu Kα radiation (λCu Kα=1.5418 Å) was collimated by a 2D Kratky collimator, and the data were collected by a 2D Pilatus 100K detector. The scattering vector Q=(4π/λ) sin θ, with 2θ being the scattering angle, was calibrated using silver behenate. Data were collected and azimuthally averaged using the Saxsgui software to yield 1D intensity vs. scattering vector Q, with a Q range from 13 to 18 nm-1. For all measurements the samples were placed inside a stainless-steel cell between two thin replaceable mica sheets and sealed by an O-ring, with a sample volume of 10 μL and a thickness of ˜1 mm.

### Methods—In Vivo Investigation

### Chemically Induced Colitis Based on Application of DSS

Female 6-8 week-old C57B/6J Mice (ex. Charles River Germany) were maintained under specific and opportunistic pathogen-free (SOPF) microbiota conditions at the animal facility of the University of Bern. Mice were ear marked, randomly assigned to different cages and treatment groups, and bedding mixed between all cages to avoid potential cage effects on microbiota. All methods used were approved by the Bernese animal welfare authority (permission no. BE 20/18). One day prior to the start of DSS supplementation, mice were intra-rectally injected with 100 μL of empty gel, TOFA in 1% methylcellulose, or gel loaded with TOFA (5 mg TOFA/100 μL gel). The next day, mouse drinking water was supplemented with 2% w/v dextran sodium sulfate (DSS; MP Biomedicals, 160110). Every other day, the different compounds were applied intra-rectally until the end of the experiment. During the experiment, the mice were constantly monitored and the weight and disease scores were recorded when appropriate. Disease score was determined by grading of 1-4 of the following criteria (with grade 4 corresponding to most unhealthy/abnormal): posture, mobility, fur appearance, weight, stool consistency, and stool colour (L. F. Mager, et al., The ESRP1-GPR137 axis contributes to intestinal pathogenesis, Elife 6 (2017), doi: 10.7554/eLife.28366.). At the termination of the experiment, mice were euthanised by asphyxia with carbon dioxide, and organs were collected and used as described in the results. Swiss rolls (C. Moolenbeek, et al., (1981) The “Swiss roll”: A simple technique for histological studies of the rodent intestine, Lab. Anim. 15, 57-59) were made from the colons, fixed overnight with 10% formalin in PBS, then washed with PBS, embedded in paraffin, and sectioned with H&E staining Histopathology scoring was performed by board-certified pathologist, in a blinded manner, using the following criteria: loss of goblet cells, crypt abscesses, epithelial erosions, hyperaemia, thickness of mucosa, and cellular infiltration (maximal score per category: 3).

### Flow Cytometry and Quantification of Single Cells

The gating strategy was adapted from the inventors previously published work (S. E. Liyanage, et al., Flow cytometric analysis of inflammatory and resident myeloid populations in mouse ocular inflammatory models, Exp. Eye Res. 151, 160-170 (2016)). Briefly, mouse spleens (after weighing) and mesenteric lymph nodes were homogenised through a 70 μm cell strainer, after which point the red blood cells were removed from the spleen by re-suspending the cell pellet in ACK lysing buffer (150 mM NH₄Cl, 10 mM KHCO₃, 0.1 mM; pH: 7.4) at room temperature for 5 minutes. Splenocytes were quantified using a CASY cell counter (Omni Life Sciences) and the following populations were quantified following single cell and live/dead selection (Thermofischer, L34961). T cells (defined as CD3ε+ cells; antibody used: eBioscience, 25-0031-82); dendritic cells (CD11c+, CD11b+; Biolegend 117324 & 101241); neutrophils (CD11b+; Ly6G+; Biolegend, B156884), Macrophages (CD11b+, CD11c−, Ly6G−, Ly6C−), and inflammatory monocytes (CD11b+, Ly6C+; Biolegend, 128024). Stained cells were analysed on a BD Bioscience LSR II SORP flow cytometer.

### T-Cell Transfer Colitis

To induce T cell mediated colitis, CD4+ T cells were isolated from the spleen of C57/BL6 mice (Strain #: 000664; RRID:IMSR_JAX: 000664 ex. Jackson laboratories) using the CD4 T cell isolation kit from Stemcell Technologies (#19852; Cologne, Germany) and subsequently naïve T helper cells (CD3+, CD4+, CD25low, CD6Lhigh, CD44low cells) were sorted on a FACS Aria III (Becton Dickinson; Eysins, Switzerland) as described previously (D. V. Ostanin, et al., T cell transfer model of chronic colitis: Concepts, considerations, and tricks of the tradeAm. J. Physiol.—Gastrointest. Liver Physiol. 296, 135-146 (2009); M. R. Spalinger, et al., PTPN2 controls differentiation of CD4+ T cells and limits intestinal inflammation and intestinal dysbiosis, Mucosal Immunol. 8, 918-929 (2015); M. R. Spalinger, et al., Loss of PTPN22 Promotes Intestinal Inflammation by Compromising Granulocyte-mediated Antibacterial Defence, J. Crohn's Colitis 15, 2118-2130 (2021)). 12-15 week old male and female Rag^{-/-} mice in the C57/BL6 background (model RAGN12 (B6.129S6-Rag2^{tm1Fwa} N12) ex. Taconic) were injected intraperitoneally with 2.5×10⁵ naïve T helper cells (all methods used were approved under licence no. ZH043/2021). Starting on day 2 post T cell injection, the mice received one daily rectal instillation (100 μL) of empty TIF-Gels, TAC-loaded TIF-Gels or TAC in vehicle solution (1% nitrocellulose in distilled water). Weight development and disease activity scores were measured on a daily basis. On the last day of the experiment (day 18), the mice were anaesthetised using a mixture of ketamine 90-120 mg/kg bodyweight (Vétoquinol, Bern, Switzerland) and xylazine 8 mg/kg bodyweight (Bayer, Lyssach, Switzerland), and were subjected to mouse endoscopy to assess the extent of endoscopic colitis (M. R. Spalinger, et al., Protein tyrosine phosphatase non-receptor type 22 modulates colitis in a microbiota-dependent manner, J. Clin. Invest. 129, 2527-2541 (2019)) using the following parameters: 1) thickening of the colon wall, 2) vascularisation/bleeding, 3) extent of fibrin deposits, 4) granular appearance of the colon wall, 5) stool consistency. Each parameter was given a score from 0 (normal) to 3 (most severe appearance), resulting in a maximal total score of 15. After colonoscopy, the mice were sacrificed and colon tissue harvested for histology and isolation of lamina propria immune cells. Immune cells were isolated from the colon, mesenteric lymph nodes and the spleen, and analysed for immune cell subsets (M. R. Spalinger, et al., (2019) Loss of PTPN22 abrogates the beneficial effect of cohousing-mediated fecal microbiota transfer in murine colitis, Mucosal Immunol. 12, 1336-1347).

### H&E Staining and Histological Analysis of Colitis Severity

To assess the microscopic extent of colitis, formalin-fixed, paraffin embedded sections of the most distal 1.5 cm of the colon were subjected to hematoxilin and eosin (H&E) staining using standard protocols (M. R. Spalinger, et al. (2019)). The sections were analysed by two blinded scientists for the extent of epithelial damage (score 0-4) and infiltration of immune cells (score 0-4) resulting in a maximal possible score of 8. Images were taken using a Zeiss Axio Imager.Z2 microscope (Zeiss), equipped with an AxioCam HRc (Zeiss, Jena, Germany) camera and ZEN imaging software (Zeiss, Germany).

Phase Transition Identification after In Vivo Applications

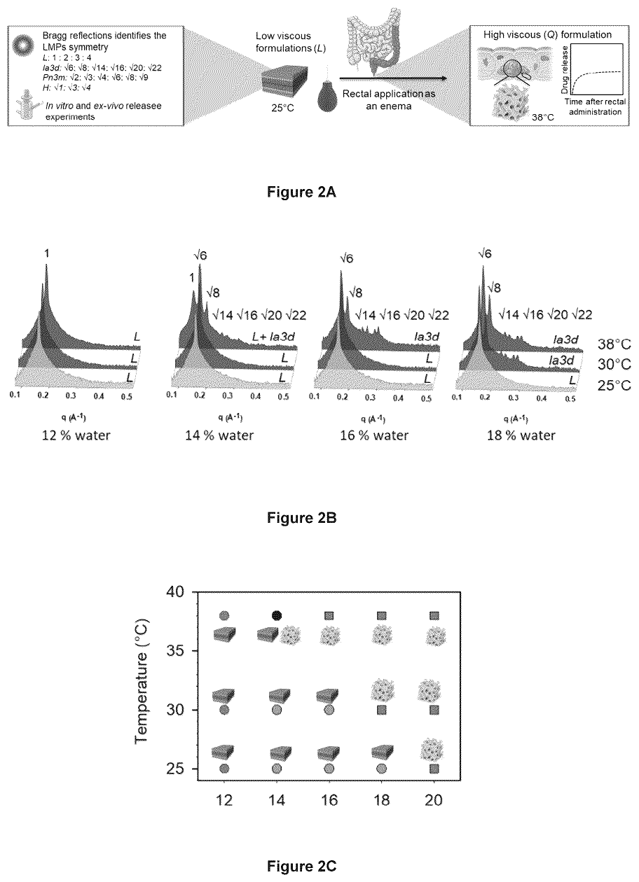

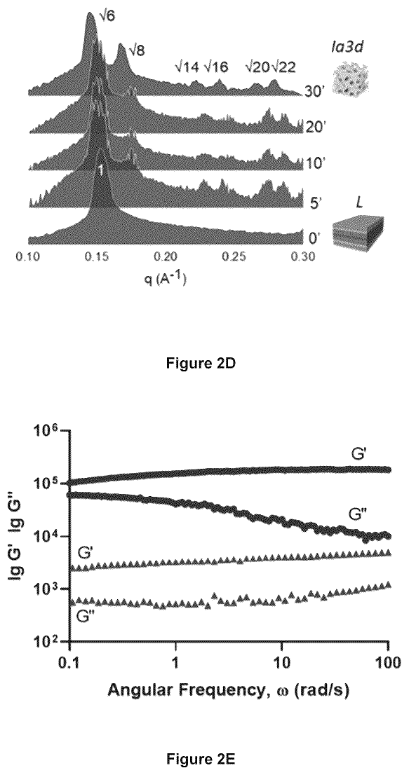

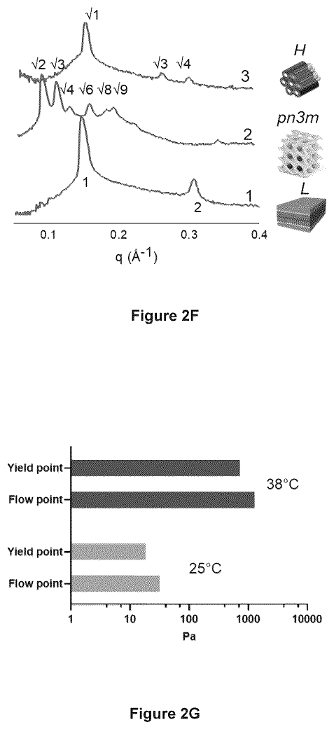

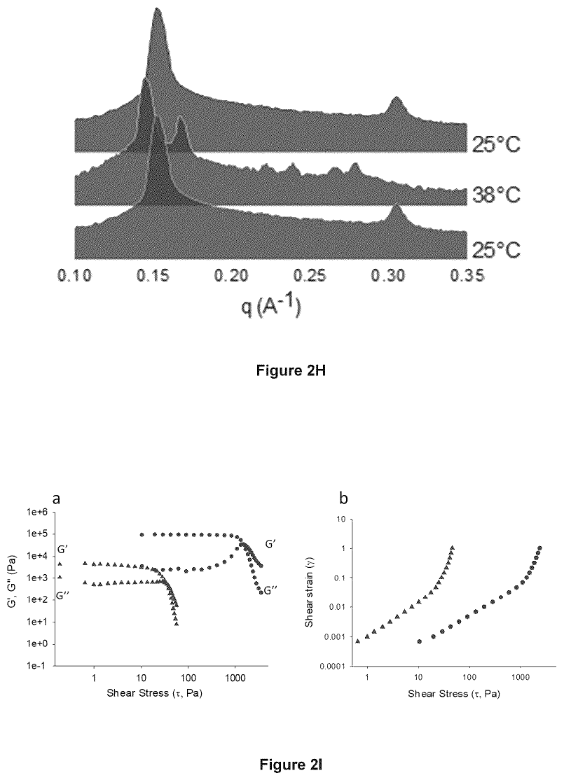

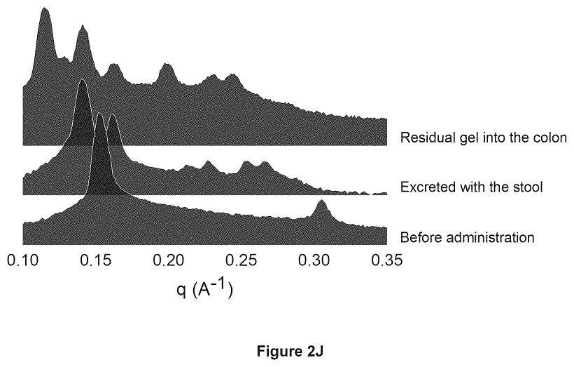

Healthy animals were administered with 100 ml of TIF-Gel and either the excreted gel (with stool after 30 min) or the residual gel present in the colon after 6 h was collected and analysed by SAXS (the animal was sacrificed, colon harvested and the residual gel washed 3× with PBS before analysis). As shown in FIG. 2J, the Bragg reflections characteristic of L phase were present before administration at 25° C., whereas the gel excreted with the stool showed the L→Ia3d transition. Moreover, the lamellar phase absorbed heat and water during the experiment reaching a cubic (pn3m) phase as was already observed in the in vitro investigations.

### Analysis of Cytokine Levels in Colon

To analyse cytokine levels in the colons, colon pieces were lysed in PBS (1 ml PBS/0.1 mg tissue) using a GentleMACS device from Miltenyi Biotec (Miltenyi Biotec, Bergisch Gladbach, Germany). Lysates were then analysed for cytokines using the Bio-Plex Pro Mouse Cytokine 23-plex Assay from Bio-Rad (Hercules, CA) according to the manufacturer's instructions.

### Pharmacokinetics (PK)

The PK studies in healthy animals were performed by the Platform of Biopharmacy of the University of Montreal, in accordance with local animal welfare committee of the University of Montreal, and in agreement with regulations of the Canadian Council on Animal Care (CCAC). Healthy female C57BL/6 mice (5 animals/group) received, under anaesthesia, a single intra-rectal administration (100 μL) of either drug-loaded TIF-Gel (TIF-Gel-TOFA or TIF-Gel-TAC) or free drugs (TOFA or TAC in suspension). All the formulations contain 5 mg of TOFA or 1 mg of TAC and they were applied once at t=0 rectally. Plasma levels were determined after 0.25, 1, 2, 4, 6, 12, 24 and 48 h post-application. Animals were euthanised with CO₂ after the last sampling point. Blood was collected and stored in K2-EDTA BD-Microtainer™ (Fisher Scientific AG, Switzerland). Drugs were extracted from plasma and their concentration was determined using LC-MS/MS analysis (see above). AUC_{0-48 h} were calculated according to the trapezoid method.

### In Vivo/Ex Vivo Experiments to Evaluate the Adhesion of the TIF-Gel to the Colon Wall

For in vivo adhesion testing, healthy animals (n=11) received an enema of 100 μl DiR (1,1′-dioctadecyl-3,3,3′,3′-tetramethylindotricarbocyanine iodide) loaded gel (DiR-TIF-Gel) under anesthesia as described above. Animals were sacrificed after 30 minutes (n=3), 2 and 6 hours (n=4). The distal 3 cm of the colon (including the rectum) was harvested and freshly imaged after a gentle washing with PBS. Intensity of the fluorescent signal was measured using the IVIS SpectrumCT In Vivo Imaging System (PerkinElmer, MA, US). DiR fluorescent signal (excitation 754 nm, emission 778 nm) was detected in the distal part of dissected colons at 3 time points post gel injection. An untreated control mouse was included in each measurement (n=3). Acquired images were analysed using the Living Image® software (PerkinElmer, MA, US). Backgrounds (untreated tissue samples) were measured for each time point. Obtained signal was analysed as radiant efficiency (RE), which is a calibrated unit that compensates for device settings and non-uniform light excitation pattern.

### Example 1—TIF-Gel Preparation

MLO was used as the lipid component of the mesophases and mixed with TOFA (5% w/w; 5 mg/100 mg) or TAC (1% w/w; 1 mg/100 mg). Lipid/drug mixtures were prepared by dissolving the appropriate amounts of lipid and drug stock solutions together in ethanol. The solvent was then completely removed under reduced pressure (freeze-drying for 24 h at 0.22 mbar) and the dried lipid mixture was hydrated by mixing weighed amounts of water (16% w/w) in sealed Pyrex tubes and alternatively centrifuging (10 min, 5000 g) several times at room temperature until a homogenous mixture was obtained. The mesophase was then equilibrated for 48 h at room temperature in the dark. The final TIF-Gel composition loaded with TOFA comprised: a) a carrier comprising: a1) water in an amount of 16% by weight of the carrier; and a2) MLO in an amount of 84% by weight of the carrier; and b) TOFA in an amount of 5% by weight of the composition. The final TIF-Gel composition loaded with TAC comprised: a) a carrier comprising: a1) water in an amount of 16% by weight of the carrier; and a2) MLO in an amount of 84% by weight of the carrier; and b) TAC in an amount of 1% by weight of the composition. The resulting TOFA concentration was 5 mg/100 mg of the composition. The resulting TAC concentration was 1 mg/100 mg of the composition. For in vivo studies, after 48 h equilibration (as described above) the formulation was loaded into a 1 ml syringe (Inkjet-F, Braun) and the dead volume of the the animal feeding needle (20G, L×diam. 1.5 in.×1.9 mm) for rectal administration was calculated so that exactly 100 μL was applied.

### Example 2—Physico-Chemical Characterisation of TAC- and TOFA-Loaded TIF-Gel

At 25° C. and in the presence of a low percentage of water, MLO forms a lamellar (L) phase with a lower structural strength with respect to the cubic phase (Q), resulting in a formulation easier to administer and able to treat remote tissue areas, as depicted in FIG. 2A. Once applied into the rectum, the precursor L phase gradually absorbs heat (and the available amount of water) from the body and rapidly (<5 min) converts into the cubic phase, contributing to the formation of a depot in situ releasing locally the incorporated drug in a sustained fashion.

Firstly, small angle X-ray scattering (SAXS) measurements were used to determine the optimal amount of water needed to obtain a lamellar phase which provides a transition to a cubic at 38° C. (FIGS. 2B and 2C). The X-ray beam directed at the gel results in a scattering pattern with a set of maxima, that correspond to sharp Bragg reflections characteristic of the long-range positional order. The sequence of Bragg reflections (and their ratio; listed in FIG. 2A) identifies the symmetry of the mesophase studied (See also FIGS. 1A and 1B).

As shown in FIGS. 2B and 2C, with 12% of water, the Bragg reflections characteristic of L phase were present at 25° C. and 38° C. Hydrating the MLO with 14% of water led to a lamellar structure at 25° C. and a coexistence of L and Q structure (with a Ia3d geometry) at 38° C., whereas increasing the amount of water up to 18% w/w induced the L→Q transition already at 30° C. On the other hand, a mesophase composed by 16% w/w of water and 84% w/w of MLO gives Bragg reflections characteristic of the lamellar structure at 25° C. and a transition to a Q structure (with a Ia3d geometry) at 38° C., i.e. the rectal temperature.

The reflections characteristic of this L phase adopt those characteristic of a Q phase after only 5 min of incubation at 38° C. (FIG. 2D), indicating a fast conversion of the lamellar precursor into the Ia3d cubic structure, particularly suited for rectal administration. The transition is reversible if the temperature is brought back to 25° C. (FIG. 2H). While this information is not relevant for rectal applications per se, it is an important property for the storage conditions of TIF-Gel.

The diverse topologies of the mesophases were confirmed by the different viscoelastic regimes identified by rheological (frequency sweep) measurements. Specifically, the precursor L phase had a low structural strength, as indicated by the lower value of storage modulus and loss modulus (G′ and G″, respectively) with respect to the viscoelastic Q phase, resulting in a less viscous pseudoplastic gel characterised by extensive energy dissipation mechanisms associated with the parallel slip of the lamellae. In simulated administration conditions, temperature increment and an increased water availability resulted in a swelling of the structure corresponding to a Q phase transition (where both G′ and G″ are higher than those obtained for L phase; FIG. 2E). Moreover, either the flow or the yield points (both representing the shear limit above which a material starts to behave like a fluid) better determine the differences between our low viscous lamellar precursor and the high viscoelastic cubic gel and they might identify a threshold above which a formulation is too elastic to be rectally applied (FIG. 2G). Since the sliding of a lamellae can occur along any possible direction, a low shear is required to be applied to this gel so that it starts behaving like a fluid and it can be forced to pass easily through a canula for enema, a syringe or a colon pipe. This translates into a low viscosity material with a low structural strength easier to administer compared to the fully hydrated cubic gel owing to its high flow and a yield point (FIG. 2I).

To prove the occurrence of the expected transition, a series of SAXS experiments were carried out after that the gel was soaked in 1 mL of HEPES (or, alternatively, in a buffer solution containing lipase) and incubated at 38° C. for 8 h. As shown in FIG. 2F, the L phase present at the beginning of the release experiment at 25° C. absorbed heat (and water) during the release experiments reaching a cubic (pn3m) phase with a lattice parameters (a=8.7 nm) and a water channel (d_{w}=4 nm) comparable with those obtained for a Pn3m phase at its maximum hydration level (43). These transitions were also confirmed in vivo where, after rectal application, the gel excreted and collected with the stool after 30 min had an Ia3d phase identity, whereas the residual gel present in the colon after 6 h was determined to be in the pn3m cubic phase (FIG. 2J).

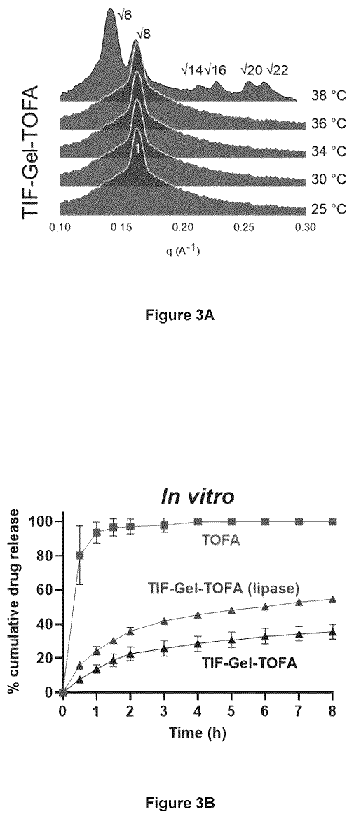

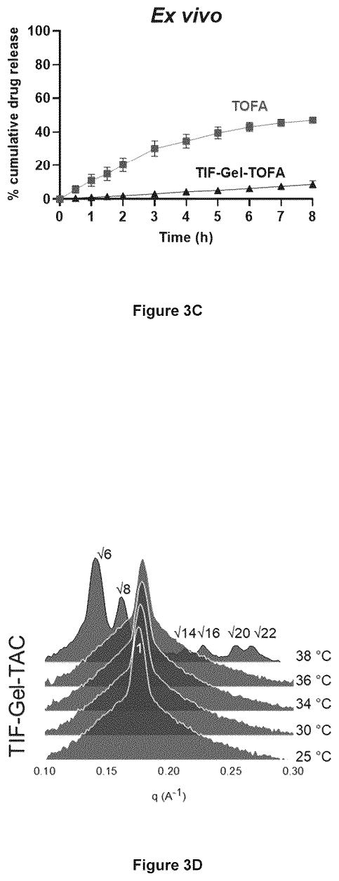

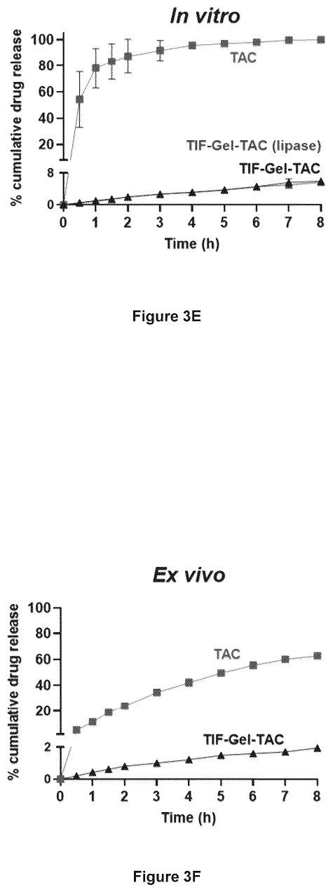

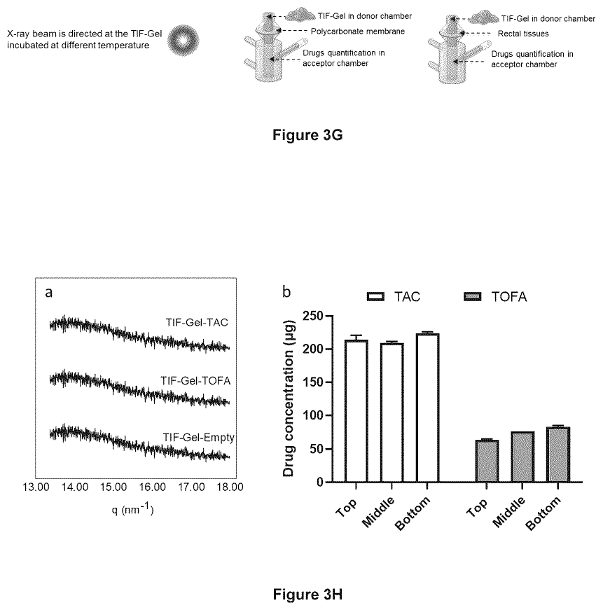

The presence of lipase (1000 U/mL) hydrolyses the ester group of MLO inducing a transition from Q→Hexagonal phase (H), with the latter not linked to any burst release phenomenon (FIG. 3). Based on this initial pivotal characterisation, the inventors chose an 84% MLO and 16% water formulation for subsequent in vitro and in vivo studies, which had suitable rheological properties to pass through a small diameter animal feeding needle (size 20G) to further expand into a sponge-like system once injected into the rectum.

### Example 3—TOFA and TAC Encapsulation and Release from TIF-Gel

To evaluate the influence of the guest drugs on the phase identity, TOFA or TAC loaded-mesophases were prepared and analysed with SAXS. The TOFA loaded mesophase composition comprised: a) a carrier comprising: a1) water in an amount of 16% by weight of the carrier; and a2) MLO in an amount of 84% by weight of the carrier; and b) TOFA in an amount of 5% by weight of the composition. The TAC loaded mesophase composition comprised: a) a carrier comprising: a1) water in an amount of 16% by weight of the carrier; and a2) MLO in an amount of 84% by weight of the carrier; and b) TAC in an amount of 1% by weight of the composition. Notably, the entrapment of drugs (5 mg of TOFA and 1 mg of TAC in 100 mg of gel-5 or 1% w/w, respectively) (FIGS. 3A and 3D) did not affect the phase identity and thermal behaviour of the carrier gel and the rectal temperature still induced a transition from L→Q phase. Both drugs could be encapsulated in the TIF-Gel with a 100% encapsulation efficacy. Moreover, both drugs do not form crystals once embedded in the 3D gel structure, as proven by the absence of reflections associated with a drug crystallisation in the wide-angle X-ray scattering (WAXS) spectra at high q (FIG. 3H). The drugs were also homogeneously distributed in the gel matrix (FIG. 3H).

When hydrated with water, the lipid/drug mixtures form the lamellar structure and the totality of the drugs are embedded in the gel. In the in vitro release experiments, drug-loaded TIF-Gel formulations were placed in the donor chamber of a vertical diffusion cell (as depicted in FIG. 3G) and kept separated from the acceptor chamber by a polycarbonate membrane with pore diameter of 3 μm, which allows the passage of the free drugs only.

Contrary to the small intestine, for which different in vitro models are established, in the case of rectum only animal models are available, mostly used in pre-clinical studies. Therefore, to bypass this limitation, an ex vivo approach was employed to study the diffusion time of the drugs from the gel across the rectal epithelium. Tissues isolated from healthy rat rectum were used as natural membrane and the amount of drugs diffused into the acceptor chamber was quantified. The 3D gel network decreased the release rate of TOFA (hydrophilic drug) either in vitro or in an ex vivo setup (FIGS. 3B and 3C, respectively). The same sets of experiments were carried out also for the TAC-loaded gel. Even in this case the gel could retain the drug and slowly release it in in vitro and ex vivo experiments (FIGS. 3E and 3F, respectively).

Notably, the presence of lipase in our experimental conditions did not induce disassembly of the gel with consequent burst release of the drug, as reported for another lipid-based gel, IT-hydrogel, developed to topically treat UC.⁴¹ The enzyme induced a responsive release (+20% of drug released) in the IT-hydrogel only after 24 h, whereas TAC and TOFA were released from our TIF-Gel within only 8 h, a time span more compatible with the retention time of rectally administered dosage forms. The structural control efficiency index (SCEI) provides an estimate of the kinetics of drug release for various phases. However, the phase identity of the gel described herein changes dynamically during the release experiment. Thus, the SCEI cannot be used to describe the release profile. Indeed, the hydrophobic drugs do not follow a Fickian diffusion profile and, consequently, the release profile cannot be modelled using the Higuchi equation. The inventors did not observe any gel erosion (no weight loss was recorded either in vitro or in ex vivo experiments) and can, therefore, reject the hypothesis that the release process is driven by gel dissolution. Interestingly, the presence of 10 mg of the drugs (10% w/w of both TOFA and TAC) did not affect the phase identity and the transition temperature of the gel, which gives a lamellar phase at room temperature and a cubic (Ia3d) phase at 38° C. (see FIGS. 1A and 1B). This demonstrates that the administration of a low volume of TIF-Gel could deliver a high amount of drug, whilst reducing the urgency associated with a high-applied volume.

### Example 4—Release of Clotrimazole, Budesonide, and Mesalamine from TIF-Gel

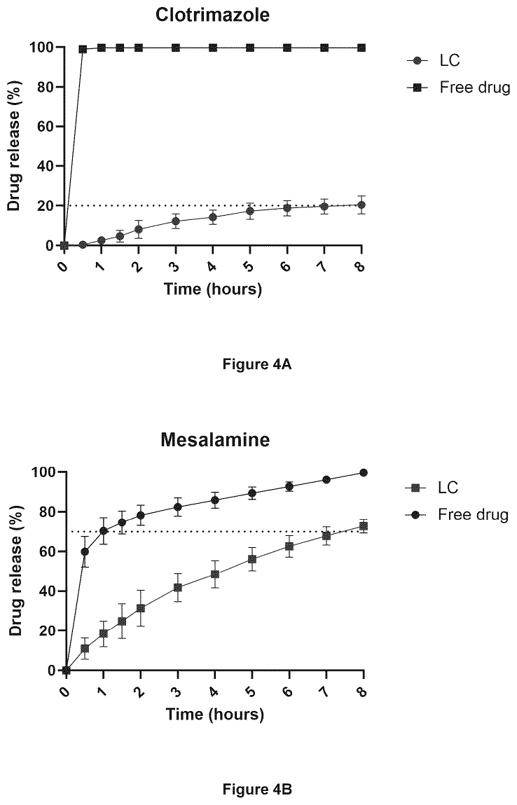

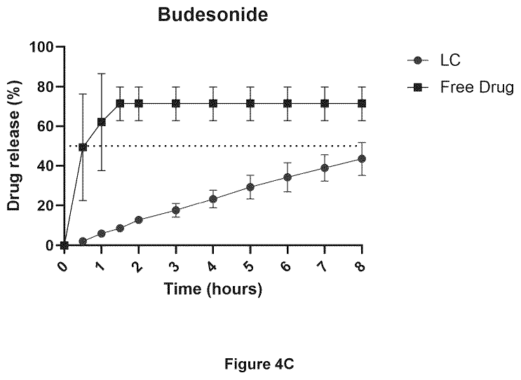

The drug-loaded TIF-Gel formulations and free drug formulations were prepared according to the methods described above in: ‘Release Experiments of Clotrimazole, Budesonide and Mesalamine: in vitro set-up and HPLC drugs quantification’. When hydrated with water, the lipid/drug mixtures form the lamellar structure and the totality of the drugs are embedded in the gel. In the in vitro release experiments, drug-loaded TIF-Gel formulations were placed in the donor chamber of a vertical diffusion cell and kept separated from the acceptor chamber by a polycarbonate membrane with pore diameter of 3 μm, which allows the passage of the free drugs only. The 3D gel network decreased the release rate of all used free drugs as shown in FIGS. 4A, 4B and 4C.

### Example 5—Effect of the TOFA/TIF-Gel on Dextran Sulfate Sodium (DSS)-Induced Acute Colitis

To test the potential efficacy of the gel in treating an acute UC flare-up, the inventors applied TIF-Gel loaded with TOFA to a mouse model of acute colitis induced by dextran sulfate sodium (DSS). The TIF-Gel loaded with TOFA comprised a) a carrier comprising: a1) water in an amount of 16% by weight of the carrier; and a2) MLO in an amount of 84% by weight of the carrier; and b) TOFA in an amount of 5% by weight of the composition. DSS is toxic to epithelial cells and its application compromises the integrity of the intestinal barrier, thereby leading to activation of submucosal immune cells by intestinal microbes. Through experimentation, it was determined that bi-daily application of the gel yielded robust mitigation of local and systemic inflammation.

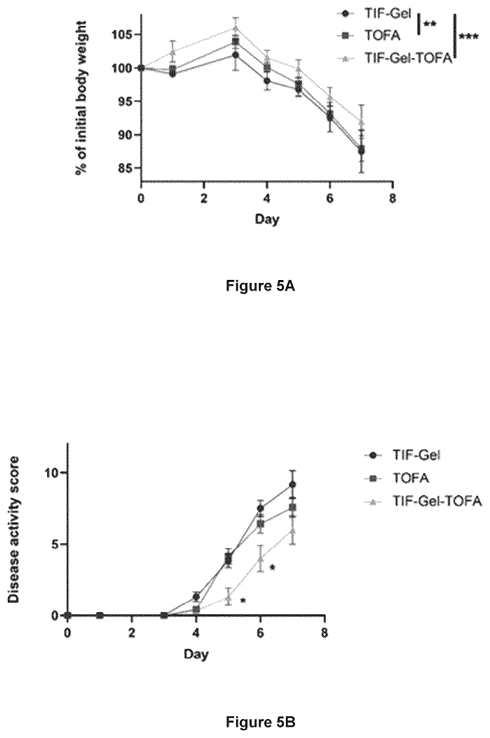

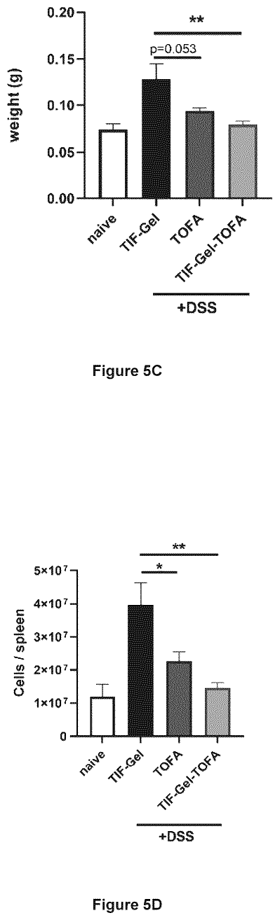

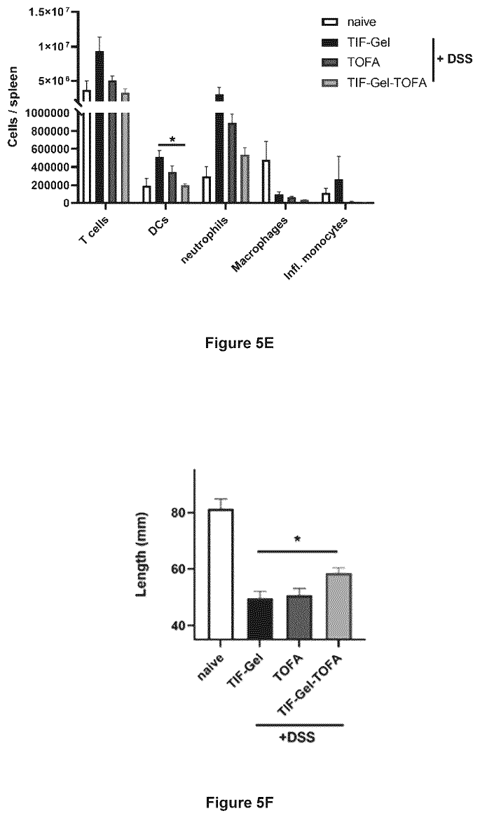

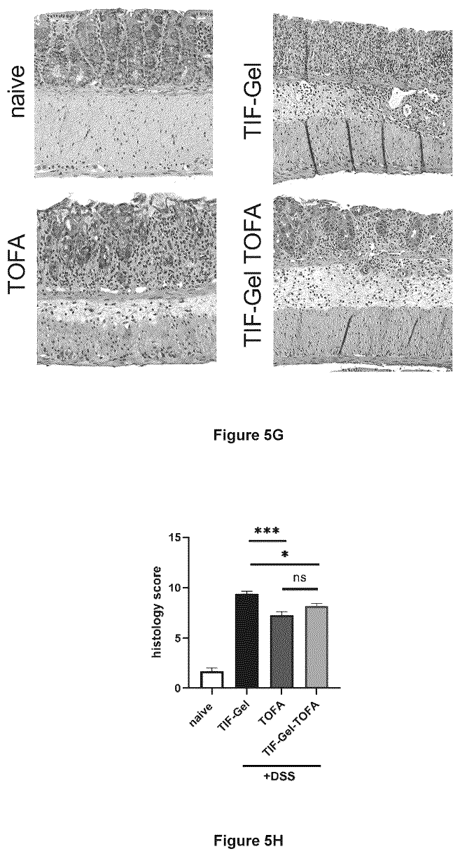

Mice treated with this regimen of TIF-Gel-TOFA displayed decreased weight loss and disease severity when compared to mice treated with an empty TIF-Gel (FIGS. 5A and 5B). In contrast, drug in vehicle solution (TOFA), while improving weight loss, did not improve the disease score in these mice (FIG. 5B). Of note, daily application of the compounds did not yield results as robust, and the differences between free TOFA and TIF-Gel-TOFA were less apparent under this regimen.

Signs of systemic inflammation, determined by spleen size and cellularity, were also alleviated in those mice treated bi-daily with the TIF-Gel-TOFA (FIGS. 5C and 5D). Furthermore, local pro-inflammatory cytokine levels were reduced in TOFA and TIF-Gel-TOFA-treated mice, and anti-inflammatory IL-10 levels were increased only in the TIF-Gel-TOFA group (FIG. 5E). Local inflammation was also mitigated by the TIF-Gel-TOFA as determined by a reduction in colon shortening and pathology (FIGS. 5F, 5G, and 5H). For colon shortening, but not for histopathology as well, TIF-Gel-TOFA was more effective than the drug in vehicle. No differences were detectable in the immune cell populations of the spleens or mesenteric lymph nodes of the different treatment groups. Overall, these data indicate that a topically applied temperature-dependent in situ-forming gel carrying TOFA represents a valuable tool to mitigate acute intestinal inflammation.

### Example 6—Effect of the TAC/TIF-Gel on T-Cell Transfer Colitis

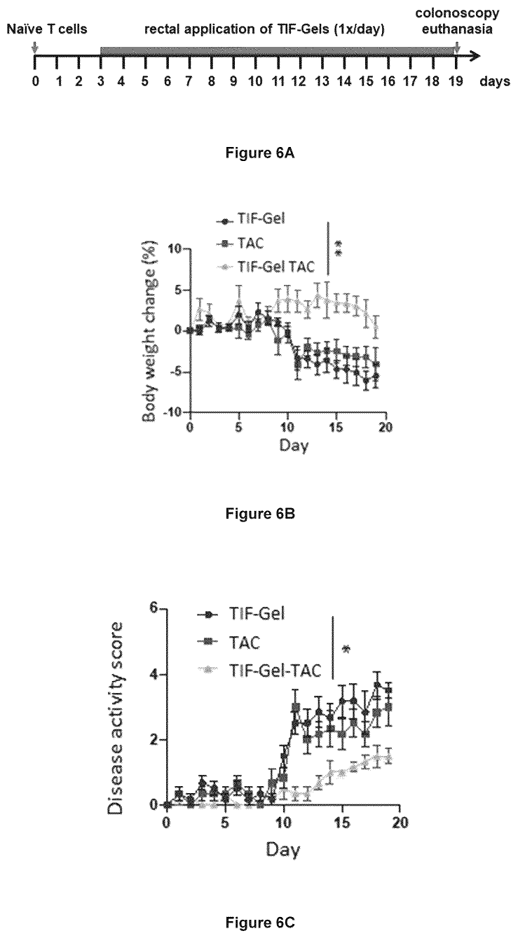

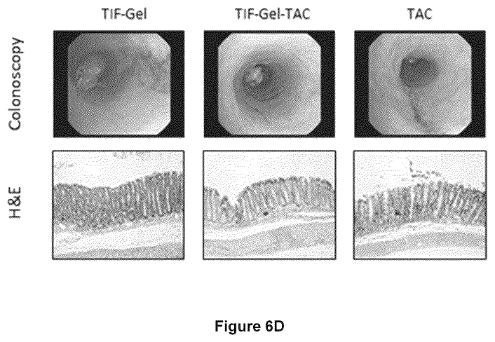

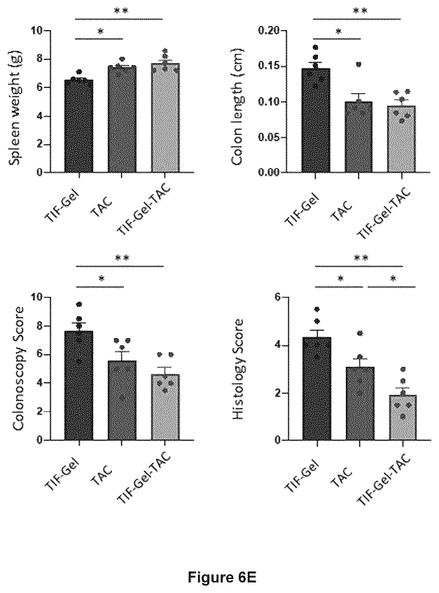

TIF-Gel acts as a platform able to host and release molecules with different polarity. Thus, the hydrophobic TAC was loaded in to TIF-Gel and its ability to reduce colitis severity was assessed also using a model of T cell mediated colitis, namely T cell transfer colitis. The TIF-Gel loaded with TAC comprised a) a carrier comprising: a1) water in an amount of 16% by weight of the carrier; and a2) MLO in an amount of 84% by weight of the carrier; and b) TAC in an amount of 1% by weight of the composition. In this model, naïve T cells are transferred into B and T cell-deficient Rag^{-/-} recipient mice, which results in the development of T cells that react against luminal antigens and subsequently strong colon inflammation. Three days after naïve T cell transfer into Rag^{-/-} hosts, mice were treated with 100 μL i) TAC-loaded TIF-Gels (TIG-Gel-TAC), ii) empty TIF-Gels (TIF-Gel) or iii) TAC in vehicle solution (TAC) via daily rectal instillation (FIG. 6A). Weight development and monitoring of disease activity demonstrated that mice that received empty TIF-Gels or TAC in vehicle solution started to develop the first signs of colitis around day 10 post T cell transfer as evidenced by progressive weight loss and signs of diarrhoea (FIGS. 6B and 6C). Of note, mice that were treated with TAC-loaded TIF-Gels did not lose weight and diarrhoea-scores were lower than in the other two groups (FIGS. 6B and 6C). On day 19 post T cell application, all mice were subjected to colonoscopy to evaluate macroscopic signs of colitis, sacrificed, and colon tissue collected for histological and molecular analyses of colitis severity. Interestingly, TAC-administration via TIF-Gels as well as TAC administration in vehicle reduced endoscopic signs of colitis. Although there was a clear trend towards further reduction of endoscopic scores in mice that received TAC in TIF-Gels, this was not significant (FIG. 6D). In contrast however, and in line with disease activity scores, mice that received TAC in TIF-Gels did not only show clearly reduced colitis severity when compared to mice that were treated with empty TIF-Gels, but also when compared to mice that received TAC in vehicle solution (FIG. 6D). Notably, all mice receiving TAC (either in TIF-Gels or administered in vehicle) showed longer colons and reduced spleen weight (FIG. 6E), indicating reduced disease in these two groups when compared to mice treated with empty TIF-Gels. Taken together, these data clearly indicate that TAC administration via TIF-Gels is superior in reducing colitis severity than TAC-administration in vehicle.

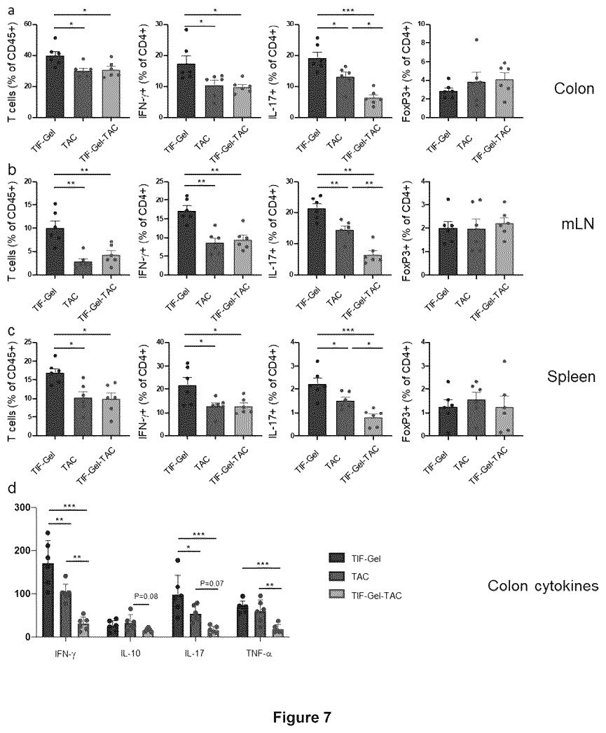

Transfer colitis is mainly mediated by aberrantly activated T helper cells, and especially IFN-γ+ (Th1) and IL-17+ (Th17) CD4+ T cells contribute to the disease. To test the effect of TAC administration either in vehicle or in the TIF-Gels, the inventors analysed proportions of T helper cells in the colonic lamina propria (FIG. 7A), mesenteric lymph nodes (FIG. 7B) and the spleen (FIG. 7C). Of note, both TAC administration forms reduced the relative abundance of T cells in the lamina propria, mesenteric lymph nodes and the spleen (FIGS. 7A to 7C). Among T helper cells, Th1 and Th17 cells were reduced with TAC in vehicle as well as with TAC-loaded TIF-Gels when compared to the mice that received empty TIF-Gels only (FIG. 7). While there was no difference among Th1 cells between mice receiving of TAC in vehicle and those receiving TAC in TIF-Gels, the reduction in Th17 cells was significantly more pronounced in mice receiving TAC in TIF-Gels than in those receiving TAC in vehicle (FIG. 7). In general, there was not much effect on the abundance of FOXP3+ (regulatory) T cells (FIG. 7). These findings were also reflected in cytokine measurements in colonic tissues (FIG. 7D), where reduced levels of IFN-γ and IL-17 in mice treated with the free drug were found. TIF-Gel-TAC further reduced levels of these two cytokines and in addition also significantly reduced levels of TNF-α (FIG. 7D), indicating that TIF-Gel-TAC was more effective at reducing production of pro-inflammatory cytokines than free drug alone. In summary, these results indicate that administration of TAC in TIF-Gels is superior in reducing disease-promoting T helper cells in the setting of T cell induced colitis.

### Example 7—Stability Study of TAC and TOFA-Loaded TIF-Gel

The TAC and TOFA-loaded TIF-Gels were prepared according to Example 1. Briefly, the final TOFA-loaded TIF-Gel composition comprised a) a carrier comprising: a1) water in an amount of 16% by weight of the carrier; and a2) MLO in an amount of 84% by weight of the carrier; and b) TOFA in an amount of 5% by weight of the composition. The TAC-loaded TIF-Gel composition comprised a) a carrier comprising: a1) water in an amount of 16% by weight of the carrier; and a2) MLO in an amount of 84% by weight of the carrier; and b) TAC in an amount of 1% by weight of the composition. The long-term stability of TOFA loaded into a gel of the invention, and TAC loaded into a gel of the invention was monitored over one month, at 4° C. and 25° C. At specific time points, an aliquot of the formulation was analysed at the HPLC, and the content of the drug was recorded. Data are expressed as relative percentage referred to day 0.

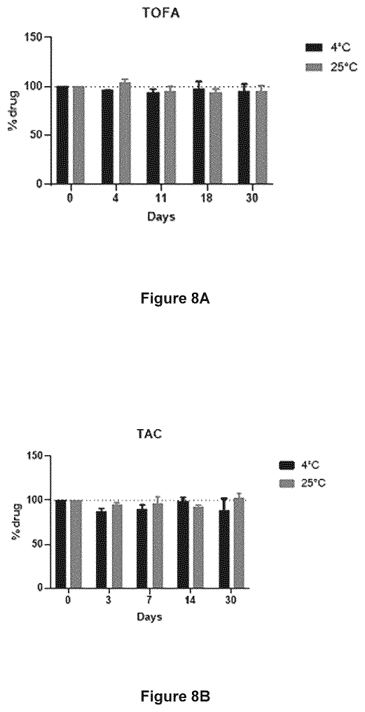

As can be seen in FIG. 8, both TOFA (FIG. 8A) and TAC (FIG. 8B) loaded gels appeared stable at both 4° C. and 25° C., over the one-month period.

### Example 8—Rectal Drug Delivery Via TIF-Gel Reduces Systemic Drug Exposure

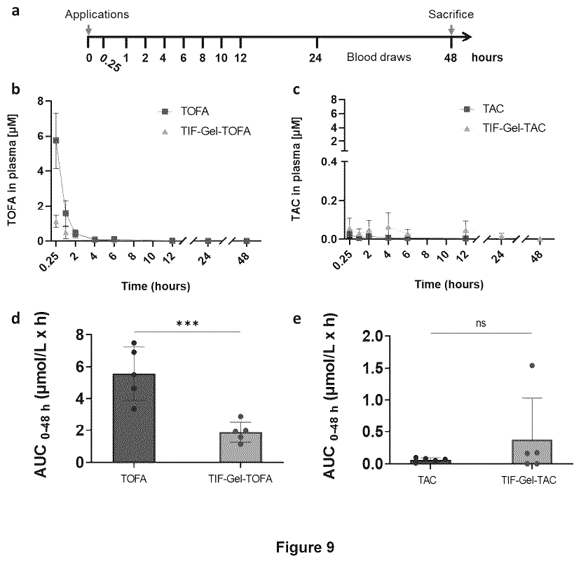

To demonstrate that rectal TIF-Gel application was indeed suitable for minimising systemic drug release, drug release was analysed in vivo by longitudinally monitoring drug plasma levels of mice after colonic TIF-Gel enema. For this purpose, healthy mice received a single enema of either drug-loaded TIF-Gel (TIF-Gel-TOFA or TIF-Gel-TAC) or of free drugs (TOFA or TAC) and plasma drug concentrations were measured at different time points (FIG. 9A). Mice receiving free TOFA had an early peak in plasma concentration at 0.25 h (FIG. 9B); TOFA plasma levels rapidly decreased thereafter, following first-order kinetics. In mice receiving TIF-Gel-TOFA, the peak concentration at 0.25 h was significantly lower. The area under the curve (AUC), a measurement of cumulative systemic drug absorption, was also significantly reduced in the mice treated with TIF-Gel-TOFA when compared to the group treated with free TOFA (FIG. 9D). Administration of TAC, either as free drug, or as drug loaded gel resulted in a low (and negligible) systemic drug circulation (FIG. 9C) and no difference was detected in their AUCs (FIG. 9E).

### Example 9—Evaluation of Adhesion of TIF-Gel to the Colon Wall

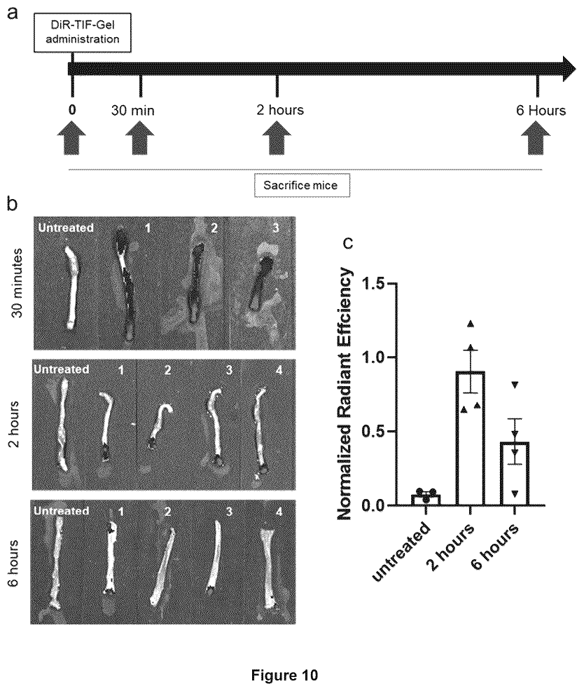

At 25° C. and in the presence of 16% w/w percentage of water, MLO forms an L phase with a low structural strength resulting in a formulation that is easy to apply and able to reach more remote areas of the colon. On the other hand, the pseudoplastic precursor has a higher viscosity than commercially available enemas such as Asacol® and Pentasa® and foam-containing 5-ASA and budesonide. Thus, once applied, the TIF-Gel adheres to healthy colonic tissue, and is retained for at least 6 hours, a time needed to avoid loss of material (see, FIG. 10).

### Additional Embodiments

The invention is further illustrated by the following numbered embodiments.

1. A composition comprising:
a) a carrier comprising:
   a1) water in an amount of more than 10% to 30% by weight of the carrier; and
   a2) lipid in an amount of 70% to 90% by weight of the carrier; and
b) a pharmaceutically active agent,
   wherein the lipid is selected from monolinolein or monoolein, and
   wherein the composition forms a lipid cubic phase at a temperature of 36° C. to 39° C.; preferably wherein the lipid is monolinolein.

2. The composition according to embodiment 1, wherein the carrier comprises from 14% to 18% water, wherein the % is % by weight of the carrier.

3. The composition according to embodiment 2, wherein the carrier comprises 16% water; wherein the % is % by weight of the carrier.

4. The composition according to any one of embodiments 1 to 3, wherein the carrier comprises from 80% to 90% lipid, wherein the % is % by weight of the carrier.

5. The composition according to embodiment 4, wherein the carrier comprises 84% monolinolein, wherein the % is % by weight of the carrier.

6. The composition according to any one of embodiments 1 to 5, wherein the composition comprises the pharmaceutically active agent in an amount of from 0.1% to 10% by weight of the composition.

7. The composition according to embodiment 6, wherein the composition comprises from 1% to 5% by weight of the composition of the pharmaceutically active agent.

8. The composition according to any one of embodiments 1 to 7, wherein the pharmaceutically active agent is a hydrophilic pharmaceutically active agent, or a hydrophobic pharmaceutically active agent.

9. The composition according to any one of embodiments 1 to 8, wherein the pharmaceutically active agent is selected from the group consisting of: AbGn168H, ABT-494, ABX464, apremilast, PF-00547659, PF-06687234, 6-mercaptopurine, adalimumab, azathioprine, bertilimumab, brazikumab (MEDI2070), cobitolimod, certolizumab pegol, CP-690,550, corticosteroids (e.g., multimax budesonide, methylprednisolone), cyclosporine, E6007, etrasimod, etrolizumab, figlotinib, guselkumab, golimumab, IL-2, IMU-838, infliximab, matrixmetalloproteinase 9 (MMP9) inhibitors (e.g., GS-5745), mesalamine, mirikizumab (LY3074828), RPC 1063, risankizumab (BI 6555066), SHP647, sulfasalazine, TD-1473, TJ301, tildrakizumab (MK 3222), tacrolimus, Janus kinase inhibitors (e.g. tofacitinib), ustekinumab, UTTR1147A, vedolizumab, immunosuppressants (e.g. rapamycin), antifibrotics (e.g. pirfenidone, nintedanib) and antifungals (e.g. clotrimazole, fluconazole).

10. The composition according to embodiment 9, wherein the pharmaceutically active agent is tofacitinib, or a pharmaceutically acceptable salt thereof.

11. The composition according to embodiment 9, wherein the pharmaceutically active agent is tacrolimus.

12. The composition according to any one of embodiments 1 to 11, wherein the composition has a lamellar phase structure at 25° C., preferably wherein the composition is a lamellar gel at 25° C.

13. The composition according to any one of embodiments 1 to 11, wherein the composition forms a lipid cubic phase at a temperature of about 38° C.

14. The composition according to any one of embodiments 1 to 13, wherein the composition further comprises an additive.

15. The composition according to any one of embodiments 1 to 14, wherein the composition is substantially free from organic solvents.

16. The composition according to any one of embodiments 1 to 15, wherein the composition has a zero shear viscosity of from 1×10⁶ to 1×10⁷ mPa·s, measured at 25° C. and 0.01 s⁻¹.

17. The composition according to any one of embodiments 1 to 16, wherein the composition is an injectable formulation.

18. The composition according to embodiment 17, wherein the injectable formulation is a subcutaneous, intramuscular or intradermal injectable formulation, preferably a subcutaneous injectable formulation.

19. The composition according to any one of embodiments 1 to 16, wherein the composition is a topical formulation.

20. The composition according to embodiment 19, wherein the topical formulation is an enema.

21. The composition according to any one of embodiments 17 to 20, wherein the formulation forms a bioadhesive controlled release depot at a temperature of 36° C. to 39° C.

22. A composition according to any one of embodiments 1 to 21, for use as a medicament.

23. A composition according to any one of embodiments 1 to 21, for use in treating a condition of the lower gastrointestinal tract.

24. The composition for use according to embodiment 23, wherein the condition is selected from the group consisting of: inflammatory bowel disease, irritable bowel disease, Crohn's disease, ulcerative colitis, colonic polyps, proctitis, radiation-associated colitis, pseudomembranous colitis, diverticulosis, diverticulitis, collagenous colitis, colorectal carcinoma and adenocarcinoma, IBD associated-perianal fistula, vaginal fistula, intestinal fibrosis, and fungal colonic infections (e.g. Paracoccidioidomycosis, histoplasmosis, and candidiasis).

25. The composition for use according to embodiment 24, wherein the condition is ulcerative colitis.

26. The composition for use according to embodiment 25, wherein the ulcerative colitis is: mild ulcerative colitis, moderate ulcerative colitis, severe ulcerative colitis, active ulcerative colitis, left-sided colitis, extensive colitis or ulcerative proctitis.

27. A composition according to any one of embodiments 1 to 16, or 19 to 21, for use in the treatment of a condition affecting the colon, wherein the composition is topically applied to the colon of a subject.

28. The composition for use according to embodiment 27, wherein the condition is selected from inflammatory bowel disease, irritable bowel disease, Crohn's disease, ulcerative colitis, colitis, pseudomembranous colitis, diverticulosis, diverticulitis, collagenous colitis and colorectal carcinoma, IBD associated-perianal fistula, vaginal fistula, intestinal fibrosis, and fungal colonic infections (e.g. Paracoccidioidomycosis, histoplasmosis, and candidiasis).

29. The composition for use according to embodiment 28, wherein the condition is ulcerative colitis.

30. The composition for use according to embodiment 29, wherein the ulcerative colitis is: mild ulcerative colitis, moderate ulcerative colitis, severe ulcerative colitis, active ulcerative colitis, left-sided colitis, extensive colitis or ulcerative proctitis.

31. The composition for use according to any one of embodiments 27 to 30, wherein the composition is administered rectally, preferably wherein the composition is administered as an enema.

32. The composition for use according to any one of embodiments 27 to 31, wherein the composition forms a controlled release depot in-situ following administration to a subject.

33. Use of a formulation comprising more than 10% w/w to 30% w/w water, and 70% w/w to 90% w/w lipid, as a carrier for a pharmaceutically active agent, wherein the lipid is selected from monolinolein or monoolein.

34. The use according to embodiment 33, wherein the pharmaceutically active agent is dispersed or dissolved in the carrier.

35. The use according to embodiment 33 or embodiment 34, wherein the carrier provides controlled release of the pharmaceutically active agent at a temperature of 36° C. to 39° C.

36. The use according to any one of embodiments 33 to 35, wherein the carrier forms a controlled release depot for the pharmaceutically active agent at a temperature of 36° C. to 39° C.

36. The use according to any one of embodiments 33 to 36, wherein the carrier is administered as an enema.

37. Use of a pre-formulation composition comprising a lipid and a pharmaceutically active agent for the manufacture of a composition according to any one of embodiments 1 to 21, wherein the lipid is selected from monolinolein or monoolein.

38. The use according to embodiment 37, wherein the pre-formulation composition is a lyophilised mixture.

39. A method of making a composition according to any one of embodiments 1 to 21, comprising:
a) hydrating a mixture comprising a lipid and a pharmaceutically active agent with water, to provide a lipid-drug mixture; and
b) equilibrating the lipid-drug mixture to provide the composition,
   wherein the lipid is selected from monolinolein or monoolein.

40. The method according to embodiment 39, wherein the mixture in step a) is a lyophilised mixture.

41. The method according to embodiment 40, wherein the lyophilised mixture is obtained by:
i) dissolving the lipid and the pharmaceutically active agent in an organic solvent; and
ii) lyophilising the mixture of i) to provide the lyophilised mixture.

42. The method according to embodiment 41, wherein in step i), the organic solvent is selected from ethanol or methanol, preferably wherein the organic solvent is ethanol.

43. A method of making a composition according to any one of embodiments 1 to 21, comprising:
a) dissolving a pharmaceutically active agent in water to provide a drug mixture;
b) hydrating a lipid with the drug mixture, to provide a lipid-drug mixture; and
c) equilibrating the lipid-drug mixture to provide the composition,
   wherein the lipid is selected from monolinolein or monoolein.

44. The method according to embodiment 43, wherein the pharmaceutically active agent is a hydrophilic pharmaceutically active agent.

45. A kit comprising:
a) a first container comprising a lipid and a pharmaceutically active agent; and
b) instructions to combine a) with water to provide the composition according to any one of embodiments 1 to 21,
   wherein the lipid is selected from monolinolein or monoolein.

46. The kit according to embodiment 45, further comprising a second container, wherein the second container comprises water.

47. The kit according to embodiment 45 or embodiment 46, wherein the lipid and the pharmaceutically active agent in the first container are provided as a lyophilised mixture.

48. A kit comprising:
a) a first container comprising a lipid; and
b) instructions to combine a) with a solution comprising a pharmaceutically active agent dissolved in water to provide the composition according to any one of embodiments 1 to 21,
   wherein the lipid is selected from monolinolein or monoolein.

49. The kit according to embodiment 48, further comprising a second container, wherein the second container comprises the pharmaceutically active agent dissolved in water.

50. The kit according to embodiment 48 or embodiment 49, wherein the pharmaceutically active agent is a hydrophilic pharmaceutically active agent.

The invention is further illustrated by the following numbered embodiments.

P1. A composition comprising:
a) a carrier comprising:
   a1) water in an amount of more than 10% to 30% by weight of the carrier; and
   a2) lipid in an amount of 70% to 90% by weight of the carrier; and
b) a pharmaceutically active agent,
   wherein the lipid is selected from monolinolein or monoolein, and
   wherein the composition forms a lipid cubic phase at a temperature of 36° C. to 39° C.;
   preferably wherein the lipid is monolinolein.

P2. The composition according to embodiment P1, wherein the carrier comprises from 14% to 18% water, wherein the % is % by weight of the carrier;
optionally wherein the carrier comprises 16% water, wherein the % is % by weight of the carrier.

P3. The composition according to embodiment P1 or embodiment P2, wherein the carrier comprises from 80% to 90% lipid, wherein the % is % by weight of the carrier;
optionally wherein the carrier comprises 84% monolinolein, wherein the % is % by weight of the carrier.

P4. The composition according to any one of embodiments P1 to P3, wherein the composition comprises the pharmaceutically active agent in an amount of from 0.1% to 10% by weight of the composition;
optionally wherein the composition comprises from 1% to 5% by weight of the composition of the pharmaceutically active agent;
further optionally wherein the pharmaceutically active agent is a hydrophilic pharmaceutically active agent, or a hydrophobic pharmaceutically active agent.

P5. The composition according to any one of embodiments P1 to P4, wherein the pharmaceutically active agent is selected from the group consisting of: AbGn168H, ABT-494, ABX464, apremilast, PF-00547659, PF-06687234, 6-mercaptopurine, adalimumab, azathioprine, bertilimumab, brazikumab (MEDI2070), cobitolimod, certolizumab pegol, CP-690,550, corticosteroids (e.g., multimax budesonide, methylprednisolone), cyclosporine, E6007, etrasimod, etrolizumab, figlotinib, guselkumab, golimumab, IL-2, IMU-838, infliximab, matrixmetalloproteinase 9 (MMP9) inhibitors (e.g., GS-5745), mesalamine, mirikizumab (LY3074828), RPC 1063, risankizumab (BI 6555066), SHP647, sulfasalazine, TD-1473, TJ301, tildrakizumab (MK 3222), tacrolimus, Janus kinase inhibitors (e.g. tofacitinib), ustekinumab, UTTR1147A, vedolizumab, immunosuppressants (e.g. rapamycin), antifibrotics (e.g. pirfenidone, nintedanib) and antifungals (e.g. clotrimazole, fluconazole),
optionally wherein the pharmaceutically active agent is:
(i) tofacitinib, or a pharmaceutically acceptable salt thereof; or
(ii) tacrolimus.

P6. The composition according to any one of embodiments P1 to P5, wherein:
(i) the composition has a lamellar phase structure at 25° C., preferably wherein the composition is a lamellar gel at 25° C.; and/or
(ii) the composition forms a lipid cubic phase at a temperature of about 38° C.; and/or
(iii) further comprises an additive; and/or
(iv) is substantially free from organic solvents; and/or
(v) has a zero shear viscosity of from 1×10⁶ to 1×10⁷ mPa·s, measured at 25° C. and 0.01 s⁻¹.

P7. The composition according to any one of embodiments P1 to P6, wherein the composition:
(i) is an injectable formulation,
   optionally wherein the injectable formulation is a subcutaneous, intramuscular or intradermal injectable formulation, preferably a subcutaneous injectable formulation; or
(ii) is a topical formulation,
   optionally wherein the topical formulation is an enema; and/or
(iii) forms a bioadhesive controlled release depot at a temperature of 36° C. to 39° C.

P8. A composition according to any one of embodiments P1 to P7, for use as a medicament.

P9. A composition according to any one of embodiments P1 to P7, for use in treating a condition of the lower gastrointestinal tract,
optionally wherein the condition is selected from the group consisting of: inflammatory bowel disease, irritable bowel disease, Crohn's disease, ulcerative colitis, colonic polyps, proctitis, radiation-associated colitis, pseudomembranous colitis, diverticulosis, diverticulitis, collagenous colitis, colorectal carcinoma and adenocarcinoma, IBD associated-perianal fistula, vaginal fistula, intestinal fibrosis, and fungal colonic infections (e.g. Paracoccidioidomycosis, histoplasmosis, and candidiasis),
further optionally wherein the condition is ulcerative colitis, for example, wherein the ulcerative colitis is selected from: mild ulcerative colitis, moderate ulcerative colitis, severe ulcerative colitis, active ulcerative colitis, left-sided colitis, extensive colitis and ulcerative proctitis.

P10. A composition according to any one of embodiments P1 to P7, for use in the treatment of a condition affecting the colon, wherein the composition is topically applied to the colon and/or rectum of a subject,
optionally wherein the condition is selected from inflammatory bowel disease, irritable bowel disease, Crohn's disease, ulcerative colitis, colitis, pseudomembranous colitis, diverticulosis, diverticulitis, collagenous colitis and colorectal carcinoma, IBD associated-perianal fistula, vaginal fistula, intestinal fibrosis, and fungal colonic infections (e.g. Paracoccidioidomycosis, histoplasmosis, and candidiasis),
further optionally wherein the condition is ulcerative colitis, for example, wherein the ulcerative colitis is selected from: mild ulcerative colitis, moderate ulcerative colitis, severe ulcerative colitis, active ulcerative colitis, left-sided colitis, extensive colitis and ulcerative proctitis.

P11. The composition for use according to embodiment P9 or embodiment P10, wherein the composition is administered rectally, optionally wherein:
(i) the composition is administered as an enema; and/or
(ii) the composition forms a controlled release depot in-situ following administration to a subject.

P12. Use of a formulation comprising more than 10% w/w to 30% w/w water, and 70% w/w to 90% w/w lipid, as a carrier for a pharmaceutically active agent,
wherein the lipid is selected from monolinolein or monoolein;
optionally wherein:
   (i) the pharmaceutically active agent is dispersed or dissolved in the carrier; and/or
   (ii) the carrier provides controlled release of the pharmaceutically active agent at a temperature of 36° C. to 39° C.; and/or
   (iii) the carrier forms a controlled release depot for the pharmaceutically active agent at a temperature of 36° C. to 39° C.; and/or
   (iv) the carrier is administered as an enema.

P13. Use of a pre-formulation composition comprising a lipid and a pharmaceutically active agent for the manufacture of a composition according to any one of embodiments P1 to P7, wherein the lipid is selected from monolinolein or monoolein, optionally wherein the pre-formulation composition is a lyophilised mixture.

P14. A method selected from Method A or Method B:

Method A: a method of making a composition according to any one of embodiments P1 to P7, comprising:
a) hydrating a mixture comprising a lipid and a pharmaceutically active agent with water, to provide a lipid-drug mixture; and
b) equilibrating the lipid-drug mixture to provide the composition,
wherein the lipid is selected from monolinolein or monoolein,
optionally wherein:
   A1) the mixture in step a) is a lyophilised mixture; and/or
   A2) the lyophilised mixture is obtained by:
      i) dissolving the lipid and the pharmaceutically active agent in an organic solvent; and
      ii) lyophilising the mixture of i) to provide the lyophilised mixture; and/or
   A3) in step i), the organic solvent is selected from ethanol or methanol, preferably wherein the organic solvent is ethanol; or

Method B: a method of making a composition according to any one of embodiments P1 to P7, comprising:
a) dissolving a pharmaceutically active agent in water to provide a drug mixture;
b) hydrating a lipid with the drug mixture, to provide a lipid-drug mixture; and
c) equilibrating the lipid-drug mixture to provide the composition,
wherein the lipid is selected from monolinolein or monoolein,
optionally wherein the pharmaceutically active agent is a hydrophilic pharmaceutically active agent.

P15. A kit selected from Kit A or Kit B:

Kit A: a kit comprising:
a) a first container comprising a lipid and a pharmaceutically active agent; and
b) instructions to combine a) with water to provide the composition according to any one of embodiments P1 to P7,
wherein the lipid is selected from monolinolein or monoolein,
optionally wherein the kit further comprises a second container, wherein the second container comprises water,
further optionally wherein the lipid and the pharmaceutically active agent in the first container are provided as a lyophilised mixture; or

Kit B: a kit comprising:
a) a first container comprising a lipid; and
b) instructions to combine a) with a solution comprising a pharmaceutically active agent dissolved in water to provide the composition according to any one of embodiments P1 to P7,
wherein the lipid is selected from monolinolein or monoolein,
   optionally wherein the kit further comprising a second container, wherein the second container comprises the pharmaceutically active agent dissolved in water,
   further optionally wherein the pharmaceutically active agent is a hydrophilic pharmaceutically active agent.

### REFERENCES

1. J. Meier, in Inflammatory Bowel Disease Nursing Manual, (Springer International Publishing, Cham, 2019), pp. 11-14
2. R. Ungaro, et al., Ulcerative colitis, Lancet 389, 1756-1770 (2017)
3. S. Singh, et al., AGA Technical Review on the Management of Moderate to Severe Ulcerative Colitis, Gastroenterology 158, 1465-1496.e17 (2020)
4. C. W. Ko, et al., AGA Clinical Practice Guidelines on the Management of Mild-to-Moderate Ulcerative Colitis, Gastroenterology 156, 748-764 (2019).
5. M. Manz, et al., Treatment algorithm for moderate to severe ulcerative colitis, Swiss Med. Wkly. 141 (2011), doi: 10.4414/smw.2011.13235
6. J. Wehkamp, et al., Recent advances and emerging therapies in the non-surgical management of ulcerative colitis, F1000Research 7, 1207 (2018)
7. R. Khanna, et al., Ustekinumab for Ulcerative Colitis, Gastroenterology 160, 2184-2186 (2021)
8. W. J. Sandborn, et al., Ozanimod as Induction and Maintenance Therapy for Ulcerative Colitis, N. Engl. J. Med. 385, 1280-1291 (2021)
9. G. D'Haens, Risks and benefits of biologic therapy for inflammatory bowel diseases, Gut 56, 725-732 (2007)
10. S. Ben-Horin, et al., Optimizing biologic treatment in IBD: objective measures, but when, how and how often?, BMC Gastroenterol. 15, 178 (2015)
11. J. Kirchgesner, et al., Risk of Serious and Opportunistic Infections Associated With Treatment of Inflammatory Bowel Diseases, Gastroenterology 155, 337-346.e10 (2018)
12. G. Mocci, et al., Dermatological adverse reactions during anti-TNF treatments: Focus on inflammatory bowel disease, J. Crohn's Colitis 7, 769-779 (2013)
13. F. Hoentjen, et al., Safety of anti-tumor necrosis factor therapy in inflammatory bowel disease, World J. Gastroenterol. 15, 2067-2073 (2009)
14. S. Danese, et al., JAK inhibition using tofacitinib for inflammatory bowel disease treatment: a hub for multiple inflammatory cytokines, Am. J. Physiol. Liver Physiol. 310, G155-G162 (2016)
15. S. Bonovas, et al., Systematic review with network meta-analysis: comparative assessment of tofacitinib and biological therapies for moderate-to-severe ulcerative colitis, Aliment. Pharmacol. Ther. (2018), doi: 10.1111/apt.14449
16. S. Singh, et al., Systematic review with network meta-analysis: first- and second-line pharmacotherapy for moderate-severe ulcerative colitis, Aliment. Pharmacol. Ther. (2018), doi: 10.1111/apt.14422
17. H. Ogata, et al., A randomised dose finding study of oral tacrolimus (FK506) therapy in refractory ulcerative colitis, Gut 55, 1255-1262 (2006)
18. G. Rogler, Gastrointestinal and liver adverse effects of drugs used for treating IBD, Best Pract. Res. Clin. Gastroenterol. (2010), doi: 10.1016/j.bpg.2009.10.011
19. R. Westhovens, Clinical efficacy of new JAK inhibitors under development. Just more of the same?, Rheumatol. (United Kingdom) 58, 127-i33 (2019)
20. K. Haga, et al., Effectiveness and Nephrotoxicity of Long-Term Tacrolimus Administration in Patients with Ulcerative Colitis, J. Clin. Med. 9, 1771 (2020)
21. S. Godat, et al., Frequency and type of drug-related side effects necessitating treatment discontinuation in the Swiss Inflammatory Bowel Disease Cohort, Eur. J. Gastroenterol. Hepatol. 30, 612-620 (2018)
22. P. Frei, et al., Topical therapies in inflammatory bowel disease, Digestion 86, 36-44 (2012)
23. R. D. Cohen, et al., A comprehensive review of topical therapies for distal ulcerative colitis, Gastroenterol. Hepatol. 16, 21-27 (2020)
24. J. K. Marshall, et al., Rectal aminosalicylate therapy for distal ulcerative colitis: a meta-analysis, Aliment. Pharmacol. Ther. 9, 293-300 (1995)
25. S. Katz, et al., in Gastroenterology and Hepatology, (Millenium Medical Publishing, 2010), vol. 6, pp. 1-12.
26. M. Safdi, et al., A Double-Blind Comparison of Oral versus Rectal Mesalamine versus Combination Therapy in the Treatment of Distal Ulcerative Colitis., Am. J. Gastroenterol. 92 (1997)
27. I. C. Lawrance, et al., Rectal tacrolimus in the treatment of resistant ulcerative proctitis, Aliment. Pharmacol. Ther. 28, 1214-1220 (2008)
28. J. M. van Dieren, et al., Local application of tacrolimus in distal colitis: Feasible and safe, Inflamm. Bowel Dis. 15, 193-198 (2009)
29. I. C. Lawrance, et al., Efficacy of Rectal Tacrolimus for Induction Therapy in Patients With Resistant Ulcerative Proctitis, Clin. Gastroenterol. Hepatol. 15, 1248-1255 (2017)
30. S. R. Fehily, et al., Simple water-based tacrolimus enemas for refractory proctitis, JGH Open 4, 561-564 (2020)
31. I. C. Lawrance, et al., Efficacy of Rectal Tacrolimus for Induction Therapy in Patients With Resistant Ulcerative Proctitis, Clin. Gastroenterol. Hepatol. (2017), doi: 10.1016/j.cgh.2017.02.027
32. I. Seoane-Viaño, et al., 3D printed tacrolimus suppositories for the treatment of ulcerative colitis, Asian J. Pharm. Sci. 16, 110-119 (2021)
33. P. Frei, et al., Rectal treatment for inflammatory bowel disease, (2017)
34. P. Gionchetti, et al., Comparison of mesalazine suppositories in proctitis and distal proctosigmoiditis, Aliment. Pharmacol. Ther. 11, 1053-1057 (1997)
35. M. Campieri, et al., 5-Aminosalicylic acid as enemas or suppositories in distal ulcerative colitis?, J. Clin. Gastroenterol. 10, 406-409 (1988)
36. F. Seibold, et al., Topical therapy is underused in patients with ulcerative colitis, J. Crohn's Colitis 8, 56-63 (2014)
37. R. Mezzenga, et al., Shear rheology of lyotropic liquid crystals: a case study, Langmuir 21, 3322-3333 (2005)
38. R. Negrini, et al., pH-Responsive Lyotropic Liquid Crystals for Controlled Drug Delivery, Langmuir 27, 5296-5303 (2011)
39. S. Hyde, in Handbook of Applied Surface and Colloid Chemistry, (John Wiley & Sons, Ltd., 2001), ch. 16, pp. 299-332.
40. J. Vallooran, et al., Lipidic Cubic Phases as a Versatile Platform for the Rapid Detection of Biomarkers, Viruses, Bacteria, and Parasites, Adv. Funct. Mater. 26, 181-190 (2016)
41. S. Zhang et al., An inflammation-targeting hydrogel for local drug delivery in inflammatory bowel disease, Sci. Transl. Med. 7, 1-10 (2015)

1. A composition for use in treating a condition of the lower gastrointestinal tract, wherein the composition comprises:
a) a carrier comprising:
a1) water in an amount of more than 10% to 30% by weight of the carrier; and
a2) monoacylglycerol lipid in an amount of 70% to 90% by weight of the carrier, wherein the monoacylglycerol lipid comprises monolinolein or monoolein, or combinations thereof; and
b) a pharmaceutically active agent,
wherein the composition forms a lipid cubic phase at a temperature of 36° C. to 39° C., and
wherein the composition is administered rectally;
preferably wherein the monoacylglycerol lipid is monolinolein.

2. The composition for use according to claim 1, wherein the carrier comprises from 14% to 18% water, wherein the % is % by weight of the carrier;
optionally wherein the carrier comprises 16% water, wherein the % is % by weight of the carrier.

3. The composition for use according to claim 1 or claim 2, wherein the carrier comprises from 80% to 90% monoacylglycerol lipid, wherein the % is % by weight of the carrier;
optionally wherein the carrier comprises 84% monolinolein, wherein the % is % by weight of the carrier.

4. The composition for use according to any one of claims 1 to 3, wherein the composition comprises the pharmaceutically active agent in an amount of from 0.1% to 10% by weight of the composition;
optionally wherein the composition comprises from 1% to 5% by weight of the composition of the pharmaceutically active agent;
further optionally wherein the pharmaceutically active agent is a hydrophilic pharmaceutically active agent, or a hydrophobic pharmaceutically active agent.

5. The composition for use according to any one of claims 1 to 4, wherein the pharmaceutically active agent is selected from the group consisting of biological agents (e.g. anti-TNF antibodies, IL-23 inhibitors, IL-12 inhibitors, TLR9 agonists, anti-MAdCAM antibodies, human IL-22Fc fusion protein, interleukins, anti-β7 integrin antibodies, matrixmetalloproteinase 9 (MMP9) inhibitors), JAK inhibitors, PDE4 inhibitors, sphingosine-1-phosphate receptor modulators, anti-inflammatory agents, corticosteroids, immunosuppressants, antifungal agents, antibiotics, antifibrotic agents, and anti-cancer agents,
optionally wherein the pharmaceutically active agent is selected from the group consisting of:
(i) ABT-494, ABX464, apremilast, 6-mercaptopurine, azathioprine, CP-690,550, multimax budesonide, methylprednisolone, cyclosporine, E6007, etrasimod, figlotinib, IMU-838, mesalamine, RPC 1063, sulfasalazine, TD-1473, TJ301, tacrolimus, tofacitinib, rapamycin, pirfenidone, nintedanib, clotrimazole, and fluconazole; and/or
(ii) AbGn168H, PF-00547659, PF-06687234, adalimumab, bertilimumab, brazikumab (MEDI2070), cobitolimod, certolizumab pegol, etrolizumab, guselkumab, golimumab, IL-2, infliximab, GS-5745, mirikizumab (LY3074828), risankizumab (BI 6555066), SHP647, tildrakizumab (MK 3222), ustekinumab, UTTR1147A, and vedolizumab,
further optionally wherein the pharmaceutically active agent is:
(a) tofacitinib, or a pharmaceutically acceptable salt thereof; or
(b) tacrolimus.

6. The composition for use according to any one of claims 1 to 5, wherein:
(i) the composition has a lamellar phase structure at 25° C., preferably wherein the composition is a lamellar gel at 25° C.; and/or
(ii) the composition forms a lipid cubic phase at a temperature of about 38° C.; and/or
(iii) the composition further comprises an additive; and/or
(iv) the composition is substantially free from organic solvents; and/or
(v) the composition has a zero shear viscosity of from 1×10⁶ to 1×10⁷ mPa·s, measured at 25° C. and 0.01 s⁻¹.

7. The composition for use according to any one of claims 1 to 6, wherein the composition:
(i) is administered as an enema; and/or
(ii) forms a bioadhesive controlled release depot at a temperature of 36° C. to 39° C.

8. The composition for use according to any one of claims 1 to 7, wherein the condition is selected from the group consisting of: inflammatory bowel disease, irritable bowel disease, Crohn's disease, ulcerative colitis, colonic polyps, proctitis, radiation-associated colitis, pseudomembranous colitis, diverticulosis, diverticulitis, collagenous colitis, colorectal carcinoma and adenocarcinoma, IBD associated-perianal fistula, vaginal fistula, intestinal fibrosis, and fungal colonic infections (e.g. Paracoccidioidomycosis, histoplasmosis, and candidiasis),
optionally wherein the condition is ulcerative colitis, for example, wherein the ulcerative colitis is selected from: mild ulcerative colitis, moderate ulcerative colitis, severe ulcerative colitis, active ulcerative colitis, left-sided colitis, extensive colitis and ulcerative proctitis.

9. A composition comprising:
a) a carrier comprising:
a1) water in an amount of from 14% to 18% by weight of the carrier; and
a2) monoacylglycerol lipid in an amount of 82% to 86% by weight of the carrier, wherein the monoacylglycerol lipid comprises at least 50% by weight monolinolein; and
b) a pharmaceutically active agent in an amount of 0.1% to 10% by weight of the composition,
wherein the composition forms a lipid cubic phase at a temperature of 36° C. to 39° C.

10. The composition according to claim 9, wherein:
(i) the composition comprises 16% water, wherein the % is % by weight of the carrier; and/or
(ii) the composition is substantially free of other lipids.

11. The composition according to claim 9 or claim 10, wherein:
(i) the composition comprises from 1% to 5% by weight of the composition of the pharmaceutically active agent;
optionally wherein the pharmaceutically active agent is a hydrophilic pharmaceutically active agent, or a hydrophobic pharmaceutically active agent; and/or
(ii) the pharmaceutically active agent is selected from the group consisting of biological agents (e.g. anti-TNF antibodies, IL-23 inhibitors, IL-12 inhibitors, TLR9 agonists, anti-MAdCAM antibodies, human IL-22Fc fusion protein, interleukins, anti-β7 integrin antibodies, matrixmetalloproteinase 9 (MMP9) inhibitors), JAK inhibitors, PDE4 inhibitors, sphingosine-1-phosphate receptor modulators, anti-inflammatory agents, corticosteroids, immunosuppressants, antifungal agents, antibiotics, antifibrotic agents, and anti-cancer agents,
optionally wherein the pharmaceutically active agent is selected from the group consisting of:
(I) ABT-494, ABX464, apremilast, 6-mercaptopurine, azathioprine, CP-690,550, multimax budesonide, methylprednisolone, cyclosporine, E6007, etrasimod, figlotinib, IMU-838, mesalamine, RPC 1063, sulfasalazine, TD-1473, TJ301, tacrolimus, tofacitinib, rapamycin, pirfenidone, nintedanib, clotrimazole, and fluconazole; and/or
(II) AbGn168H, PF-00547659, PF-06687234, adalimumab, bertilimumab, brazikumab (MEDI2070), cobitolimod, certolizumab pegol, etrolizumab, guselkumab, golimumab, IL-2, infliximab, GS-5745, mirikizumab (LY3074828), risankizumab (BI 6555066), SHP647, tildrakizumab (MK 3222), ustekinumab, UTTR1147A, and vedolizumab,
further optionally wherein the pharmaceutically active agent is:
(a) tofacitinib, or a pharmaceutically acceptable salt thereof; or
(b) tacrolimus.

12. The composition according to any one of claims 9 to 11, wherein the composition:
(i) is an injectable formulation,
optionally wherein the injectable formulation is a subcutaneous, intramuscular or intradermal injectable formulation, preferably a subcutaneous injectable formulation; or
(ii) is a topical formulation,
optionally wherein the topical formulation is an enema; and/or
(iii) forms a bioadhesive controlled release depot at a temperature of 36° C. to 39° C.

13. Use of a pre-formulation composition comprising the monoacylglycerol lipid and the pharmaceutically active agent for the manufacture of a composition according to any one of claims 9 to 12,
optionally wherein the pre-formulation composition is a lyophilised mixture.

14. A method selected from Method A, Method B, or Method C:
Method A: a method of making a composition for use according to any one of claims 1 to 8, or a composition according to any one of claims 9 to 12, comprising:
a) hydrating a mixture comprising the lipid and the pharmaceutically active agent with water, to provide a lipid-drug mixture; and
b) equilibrating the lipid-drug mixture to provide the composition,
optionally wherein:
A1) the mixture in step a) is a lyophilised mixture; and/or
A2) the lyophilised mixture is obtained by:
i) dissolving the lipid and the pharmaceutically active agent in an organic solvent; and
ii) lyophilising the mixture of i) to provide the lyophilised mixture; and/or
A3) in step i), the organic solvent is selected from ethanol or methanol, preferably wherein the organic solvent is ethanol; or
Method B: a method of making a composition for use according to any one of claims 1 to 8, or a composition according to any one of claims 9 to 12, comprising:
a) dissolving the pharmaceutically active agent in water to provide a drug mixture;
b) hydrating the lipid with the drug mixture, to provide a lipid-drug mixture; and
c) equilibrating the lipid-drug mixture to provide the composition,
optionally wherein the pharmaceutically active agent is a hydrophilic pharmaceutically active agent; or
Method C: a method of making a composition for use according to any one of claims 1 to 8, or a composition according to any one of claims 9 to 12, comprising:
a) heating the lipid to provide a molten lipid;
b) mixing the molten lipid with the pharmaceutically active agent, to provide a lipid-drug mixture;
c) mixing the lipid-drug mixture with water; and
d) equilibrating the lipid-drug mixture and water to provide the composition,
optionally wherein:
C₁) the molten lipid and the pharmaceutically active agent in step b) is mixed at a temperature of about 30° C. to 70° C., preferably about 40° C. to 60° C.; and/or
C₂) the lipid-drug mixture in step c) is mixed with water in a dual-syringe.

15. A kit selected from Kit A or Kit B:
Kit A: a kit comprising:
a) a first container comprising a lipid and a pharmaceutically active agent; and
b) instructions to combine a) with water to provide the composition for use according to any one of claims 1 to 8, or the composition according to any one of claims 9 to 12,
optionally wherein the kit further comprises a second container, wherein the second container comprises water,
further optionally wherein the lipid and the pharmaceutically active agent in the first container are provided as a lyophilised mixture; or
Kit B: a kit comprising:
a) a first container comprising a lipid; and
b) instructions to combine a) with a solution comprising a pharmaceutically active agent dissolved in water to provide the composition for use according to any one of claims 1 to 8, or the composition according to any one of claims 9 to 12,
optionally wherein the kit further comprising a second container, wherein the second container comprises the pharmaceutically active agent dissolved in water,
further optionally wherein the pharmaceutically active agent is a hydrophilic pharmaceutically active agent.
